(19) United States
(12) Patent Application Publication
(10) Pub. No.: US 2018/0291436 A1
(43) Pub. Date: Oct. 11, 2018

(54) NUCLEIC ACID CAPTURE METHOD AND KIT

(71) Applicant: MyOmicsDx, Inc, Towson, MD (US)

(72) Inventors: Qing WANG, Owings Mills, MD (US); Ming ZHANG, Owings Mills, MD (US)

(73) Assignee: MyOmicsDx, Inc, Towson, MD (US)

(21) Appl. No.: 15/911,161

(22) Filed: Mar. 4, 2018

Related U.S. Application Data
(63) Continuation of application No. PCT/US2018/019788, filed on Feb. 26, 2018.
Application No. PCT/US2018/019788 is a continuation-in-part of application No. 15/908,190, filed on Feb. 28, 2018.
(60) Provisional application No. 62/482,189, filed on Apr. 6, 2017.

(51) Int. Cl. C12Q 1/6834; B01J 19/00; B01L 3/00; B01L 7/00

(52) U.S. Cl. CPC C12Q 1/6834; B01L 2200/0647; B01L 3/502715; B01L 3/502761; B01L 7/52; B01J 2219/00299; B01J 2219/00335; B01J 2219/00795; B01J 2219/00522; B01J 2219/00608; B01J 2219/0061; B01J 2219/00612; B01J 2219/00623; B01J 2219/00648; B01J 2219/00722; B01J 19/0093

(57) ABSTRACT

A kit and a method for enriching target nucleic acid sequences from a biological sample are disclosed. The method includes preparing, and contacting with the biological sample, a first RNA probe set and a second RNA probe set respectively targeting both of the two antiparallel strands of a duplex segment in each target nucleic acid sequence. Each RNA probe in the first RNA probe set and the second RNA probe set can be generated by chemical synthesis or by in vitro or in vivo transcription, and can be biotin-labelled to thereby allow capturing of the target nucleic acid sequences by magnetic beads labelled with streptavidin, or can be engineered to a microfluidic channel to facilitate the capturing. The method can be applied to capture double-stranded nucleic acid sequences or single-stranded nucleic acid sequences having duplex segments, and the nucleic acid sequences can include DNAs, RNAs, or DNA-RNA hybrid molecules.

### CROSS-REFERENCE TO RELATED APPLICATION

The present application claims benefit of U.S. Provisional Application No. 62/482,189 filed on Apr. 6, 2017, is a continuation of PCT Application No. PCT/US2018/019788 filed on Feb. 26, 2018, and is a continuation-in-part, and claims priority, of U.S. Non-provisional application Ser. No. 15/908,190 filed on Feb. 28, 2018, which is a continuation of PCT Application No. PCT/US2018/016778 filed on Feb. 4, 2018, the disclosures of which are hereby incorporated by reference in their entirety.

### REFERENCE TO SEQUENCE LISTING SUBMITTED ELECTRONICALLY

The content of the electronically submitted sequence listing, file name Capture_SEQ_ST25.txt, size 190,905 bytes, and date of creation Mar. 4, 2018, filed herewith, is incorporated herein by reference in its entirety.

### TECHNICAL FIELD

This present disclosure relates generally to the area of diagnostics and prognostics, specifically to the field of biomarker technologies, more specifically to nucleic acid assays for genetic and genomic analysis, and in more particular to a method and a kit for targeted enrichment of nucleic acid sequences in nucleic acid assays.

### BACKGROUND

Genome sequencing, especially the recent next generation sequencing (NGS) technology, has emerged as a tool for global analysis of all genetic information behind a disease or an individual. For example, NGS has been widely used clinically for disease diagnostics, companion diagnostics for personalized therapeutics and disease monitoring.

### SUMMARY OF THE INVENTION

In a first aspect, the disclosure provides a kit for enriching at least one target nucleic acid sequence from a biological sample.

According to some embodiments, the kit comprises at least one pair of RNA probe sets and a solid support labelled with a coupling partner on a surface thereof. Each pair of RNA probe sets comprises a first RNA probe set and a second RNA probe set configured to respectively target two antiparallel strands of a duplex segment in each of the at least one target nucleic acid sequence, wherein each RNA probe in any of the first RNA probe set and the second RNA probe set is labelled with an immobilization portion. The coupling partner is configured to be able to form a secure coupling to the immobilization portion to thereby allow immobilization of each RNA probe in any of the first RNA probe set and the second RNA probe set in the each of the at least one pair of RNA probe sets onto the solid support.

Herein, each RNA probe in any of the first RNA probe set and the second RNA probe set in the each of the at least one pair of RNA probe sets can have a length of about 100-150 nt.

In the kit, the first RNA probe set and the second RNA probe set in the each of the at least one pair of RNA probe sets can be configured to respectively target a substantially same portion, or different portions, of the duplex segment in the each of the at least one target nucleic acid sequence.

According to some embodiments of the kit, the immobilization portion can comprise a biotin moiety, the solid support comprises at least one of a magnetic bead, a filter, a resin bead, a nanosphere, a plastic surface, a microtiter plate, a glass surface, a slide, a membrane, a microfluidic channel, a chip, or a matrix, and the solid support can be labelled with at least one of streptavidin, avidin, or an anti-biotin antibody.

According to some embodiments, the kit further comprises an apparatus having a working surface as the solid support. The first RNA probe set and the second RNA probe set in each pair are respectively conjugated onto the working surface, arranged such that each RNA probe in the solid support-conjugated first RNA probe set does not substantially interact with each RNA probe in the solid support-conjugated second RNA probe set. Herein the apparatus can be one of a column, a microfluidic channel, or a chip.

Optionally, the solid support-conjugated first RNA probe set and the solid support-conjugated second RNA probe set in each pair can be respectively arranged at at least one, and preferably more than one, pair of two different regions of the working surface of the apparatus.

Optionally, the solid support-conjugated first RNA probe set and the solid support-conjugated second RNA probe set in each pair can be mixedly arranged on the working surface of the apparatus, configured such that each RNA probe from the first RNA probe set has a relatively large distance to each RNA probe from the second RNA probe set to thereby substantially prevent an interaction therebetween.

In any of the above embodiments, the apparatus can be configured to allow the biological sample to flow sequentially through the working surface for more than one round.

In the biological sample, one or more of the at least one target nucleic acid sequence are each in a polynucleotide comprising at least one un-targeted sequence. As such, the kit can further include at least one blocking oligo, configured to respectively hybridize with, and to thereby block, at least one strand of each of the at least one un-targeted sequence in the polynucleotide. If the at least one un-targeted sequence in the polynucleotide comprises a first adaptor sequence and a second adaptor sequence flanking each of the one or more of the at least one target nucleic acid sequence, the at least one blocking oligo can be configured to respectively block one strand of the first adaptor sequence and one strand of the second adaptor sequence in the polynucleotide, or to respectively block both two antiparallel strands of the first adaptor sequence and both two antiparallel strands of the second adaptor sequence in the polynucleotide. As such, in the kit, the at least one blocking oligo can comprise a first blocking oligo set and a second blocking oligo set, configured to respectively block two antiparallel strands of one of the first adaptor sequence and the second adaptor sequence in the polynucleotide. The first blocking oligo set and the second blocking oligo set can be configured to respectively target two different portions within the one of the first adaptor sequence and the second adaptor sequence in the polynucleotide.

According to some other embodiments, the kit can include a plurality of DNA vectors, NTPs comprising each of ATPs, UTPs, GTPs, and CTPs; immobilization portion-labelled NTPs, wherein NTPs comprises at least one of ATPs, UTPs, GTPs, and CTPs; and a solid support labelled with a coupling partner on a surface thereof. The plurality of DNA vectors comprises at least one pair of DNA vectors, each pair comprising a first DNA vector and a second DNA vector configured, via transcription thereover, to respectively obtain a first RNA probe set and a second RNA probe set targeting respectively two antiparallel strands of a duplex segment in each of the at least one target nucleic acid sequence. The coupling partner is configured to be able to form a secure coupling to the immobilization portion.

Herein, the immobilization portion can include a biotin moiety, and the NTPs labelled with the immobilization portion can comprise biotin-labelled UTPs, having a relative molar percentage of 2%-100% among all UTPs in the kit. The solid support can comprise comprises at least one of a magnetic bead, a filter, a resin bead, a nanosphere, a plastic surface, a microtiter plate, a glass surface, a slide, a membrane, a microfluidic channel, a chip, or a matrix, and the solid support can be labelled with at least one of streptavidin, avidin, or an anti-biotin antibody.

According to some embodiments, the kit can further include an RNA ligase, comprising at least one of T4 RNA ligase or CircLigase RNA ligase, which is configured to ligate a 3′ hydroxyl terminus of each RNA probe in any of the first RNA probe set and the second RNA probe set generated from each of the at least one pair of DNA vectors with one of the immobilization portion-labeled NTPs.

Each of the plurality of DNA vectors may comprise a DNA template and a promoter. The DNA template may comprise a sequence corresponding to one of two antiparallel strands of a duplex segment in each of the at least one target nucleic acid sequence. The promoter can be configured to initiate a transcription reaction of the DNA template in a presence of an RNA polymerase compatible with the promoter. The promoter is selected from one of a T3 promoter, a T7 promoter, a SP6 promoter, and the kit can correspondingly further comprise a T3 RNA polymerase, a T7 RNA polymerase, or a SP6 RNA polymerase, corresponding to the promoter in each of the plurality of DNA vectors.

The kit can further comprise cells or viruses containing the RNA polymerase compatible with the promoter in each of the plurality of DNA vectors. The cells can include at least one of a bacterial cell line, a yeast cell line, or a mammalian cell line. Depending on the different host cells or host viruses, the promoter can be any of the T3 promoter, T7 promoter, and SP6 promoter, and can optionally be a tissue or cell line-specific promoter. There are no limitations herein.

In the kit, each of the plurality of DNA vectors can be a double-stranded DNA vector or a single-stranded DNA vector.

In the biological sample, one or more of the at least one target nucleic acid sequence are each in a polynucleotide comprising at least one un-targeted sequence. As such, the kit can further include at least one blocking oligo, configured to respectively hybridize with, and to thereby block, at least one strand of each of the at least one un-targeted sequence in the polynucleotide. If the at least one un-targeted sequence in the polynucleotide comprises a first adaptor sequence and a second adaptor sequence flanking each of the one or more of the at least one target nucleic acid sequence, the at least one blocking oligo can be configured to respectively block one strand of the first adaptor sequence and one strand of the second adaptor sequence in the polynucleotide, or to respectively block both two antiparallel strands of the first adaptor sequence and both two antiparallel strands of the second adaptor sequence in the polynucleotide. As such, in the kit, the at least one blocking oligo can comprise a first blocking oligo set and a second blocking oligo set, configured to respectively block two antiparallel strands of one of the first adaptor sequence and the second adaptor sequence in the polynucleotide. The first blocking oligo set and the second blocking oligo set can be configured to respectively target two different portions within the one of the first adaptor sequence and the second adaptor sequence in the polynucleotide.

In a second aspect, this disclosure further provides a method for enriching at least one target nucleic acid sequence from a biological sample utilizing the kit according to any one of the embodiments as described above. The method comprises the following two steps:

(1) preparing at least one pair of RNA probe sets, each pair comprising a first RNA probe set and a second RNA probe set configured to respectively target two antiparallel strands of a duplex segment in each of the at least one target nucleic acid sequence, wherein each RNA probe in any of the first RNA probe set and the second RNA probe set is labelled with an immobilization portion configured to allow immobilization onto a solid support; and

(2) capturing each strand of the at least one target nucleic acid sequence from the biological sample through hybridization of both the first RNA probe set and the second RNA probe set in the each of the at least one pair of RNA probe sets respectively with the two antiparallel strands of the duplex segment in the each of the at least one target nucleic acid sequence and through immobilization onto the solid support via the immobilization portion labelled onto each RNA probe in any of the first RNA probe set and the second RNA probe set.

Herein each of the at least one target nucleic acid sequence can be a double-stranded nucleic acid molecule, such as a double-stranded DNA molecule, a double-stranded RNA molecule, or a DNA-RNA hybrid molecule, or can be a single-stranded nucleic acid molecule (i.e. DNA or RNA) having a hairpin structure. As such, by means of the at least one pair of RNA probe sets where a first RNA probe set and a second RNA probe set in each pair respectively target two antiparallel strands of a duplex segment in each target nucleic acid sequence, each strand of the at least one target nucleic acid sequence can be captured or enriched from the biological sample.

For a typical example, each of the at least one target nucleic acid sequence in the biological sample can be a double-stranded DNA molecule, which has a plus strand and a minus strand that runs antiparallelly to form a duplex. By means of the at least one pair of RNA probe sets where a first RNA probe set and a second RNA probe set in each pair respectively target both of the two antiparallel strands (i.e. the plus strand and the minus strand) of each target nucleic acid sequence, each strand, including both the plus strand and the minus strand, of each target DNA molecule, can be captured or enriched from the biological sample.

Herein, according to some embodiments of the method, one or more of the at least one pair of RNA probe sets may be prepared through chemical synthesis. As such, the step (1) preparing at least one pair of RNA probe sets can comprise: performing chemical synthesis reactions to thereby obtain the one or more of the at least one pair of RNA probe sets. Each RNA probe in each of the one or more of the at least one pair of RNA probe sets can be labelled with the immobilization portion during the chemical synthesis reactions. Herein, the immobilization portion is covalently attached onto the solid support (i.e. each RNA probe is synthesized directly on the solid support conjugated with the immobilization portion thereof). Alternatively, each RNA probe in each of the one or more of the at least one pair of RNA probe sets can be labelled with the immobilization portion after the chemical synthesis reactions, and as such the step (1) preparing at least one pair of RNA probe sets further comprises: performing labelling reactions such that each RNA probe in each of the one or more of the at least one pair of RNA probe sets is labelled with the immobilization portion.

According to some other embodiments of the method, one or more of the at least one pair of RNA probe sets is prepared through transcription. As such, the step (1) preparing at least one pair of RNA probe sets can comprise: performing transcription reactions to thereby obtain the one or more of the at least one pair of RNA probe sets.

Herein, the sub-step of performing transcription reactions to thereby obtain the one or more of the at least one pair of RNA probe sets can include: performing transcription reactions such that each RNA probe in any of the one or more of the at least one pair of RNA probe sets is labelled with the immobilization portion during each of the transcription reactions. According to some embodiments, each of the transcription reactions is performed in presence of NTPs labelled with the immobilization portion, where the NTPs comprises at least one of ATPs, UTPs, GTPs, and CTPs. Herein the NTPs labelled with the immobilization portion can preferably comprise biotin-labelled UTPs, and the biotin-labelled UTPs can have a relative molar percentage of 2%-100% in all UTPs present in each of the transcription reactions.

Alternatively, each RNA probe in any of the one or more of the at least one pair of RNA probe sets is labelled with the immobilization portion after each of the transcription reactions. As such, the sub-step of performing transcription reactions to thereby obtain the one or more of the at least one pair of RNA probe sets can include: performing the transcription reactions; and performing a labelling.

According to some embodiments, the above sub-step of performing the transcription reactions can comprise:

providing a plurality of DNA vectors, comprising at least one pair of DNA vectors, each pair comprising a first DNA vector and a second DNA vector configured to respectively allow transcription of a first RNA molecule and a second RNA molecule respectively targeting two antiparallel strands of a duplex segment in each of the one or more of the at least one target nucleic acid sequence; and

performing the transcription reactions over the plurality of DNA vectors.

Herein, each of the plurality of DNA vectors can include a promoter, selected from one of a T3 promoter, a T7 promoter, or a SP6 promoter. At least one of the transcription reactions can be performed in vitro, or in vivo.

According to some embodiments of the method, the sub-step of performing transcription reactions over the plurality of DNA vectors comprises:

pooling the plurality of DNA vectors to obtain at least two DNA vector pools, such that the first DNA vector and the second DNA vector in the each pair of DNA vectors are not in a same DNA vector pool; and

performing a transcription reaction over each of the at least two DNA vector pools respectively to obtain RNA molecules corresponding to the each of the at least two DNA vector pools.

After the sub-step of performing a transcription reaction over each of the at least two DNA vector pools respectively and prior to the sub-step of performing a labelling, the method can further comprise: performing a fragmentation reaction to the RNA molecules corresponding to the each of the at least two DNA vector pools.

According to some other embodiments of the method, rather than pooling the DNA vectors before transcription, the sub-step of performing transcription reactions over the plurality of DNA vectors comprises: performing a transcription reaction over each of the plurality of DNA vectors to thereby obtain an RNA molecule corresponding thereto.

As such, after the sub-step of performing a transcription reaction over each of the plurality of DNA vectors and prior to the sub-step of performing a labelling, the method can further include:

pooling the RNA molecule corresponding to each of the plurality of DNA vectors to obtain at least two RNA pools, such that a pair of RNA molecules respectively targeting two antiparallel strands of a duplex segment in any one of the one or more of the at least one target nucleic acid sequence are not in a same RNA pool; and

performing a fragmentation reaction to each of the at least two RNA pools respectively.

Alternatively, after the sub-step of performing a transcription reaction over each of the plurality of DNA vectors and prior to the sub-step of performing a labelling, the method can further include:

performing a fragmentation reaction to the RNA molecule corresponding to each of the plurality of DNA vectors respectively to obtain fragmented RNA molecules corresponding to each of the plurality of DNA vectors; and

pooling the fragmented RNA molecules corresponding to each of the plurality of DNA vectors such that a pair of fragmented RNA molecules respectively targeting two antiparallel strands of a duplex segment in any one of the one or more of the at least one target nucleic acid sequence are not in a same RNA probe set.

In any of the embodiments of the method as described above, the sub-step of performing a labelling can comprise: performing ligation reactions such that an immobilization portion-labelled nucleotide is ligated to one terminus, or to a middle, of each RNA probe in each of the at least one pair of RNA probe sets. In one example, in each of the ligation reactions, a 5′ phosphate terminus of a biotin-labeled nucleotide can be ligated to a 3′ hydroxyl terminus of each RNA probe in each of the at least one pair of RNA probe sets, and each of the ligation reactions can be performed by means of an RNA ligase, which can comprise at least one of T4 RNA ligase, or CircLigase RNA ligase.

According to some embodiments, the step of preparing at least one pair of RNA probe sets comprises: performing direct transcription on the solid support to thereby obtain the one or more of the at least one pair of RNA probe sets. This can be done, for example, by means of a RNA polymerase attached on the solid support.

In the method disclosed herein, the step (2) of capturing each strand of the at least one target nucleic acid sequence from the biological sample can comprise the following sub-steps:

contacting both the first RNA probe set and the second RNA probe set in the each of the at least one pair of RNA probe sets with the at least one target nucleic acid sequence in the biological sample; and

immobilizing the at least one target nucleic acid sequence on the solid support.

Herein, each RNA probe in any of the first RNA probe set and the second RNA probe set in the each of the at least one pair of RNA probe sets can have a length of about 100-150 nt. As such, the sub-step of contacting both the first RNA probe set and the second RNA probe set in the each of the at least one pair of RNA probe sets with the at least one target nucleic acid sequence in the biological sample can be performed at a temperature of about 62-70° C., and preferably at a temperature of about 67.5° C., and for about 6-24 hours, and preferably for about 12 hours.

According to some embodiments of the method, the first RNA probe set and the second RNA probe set in the each of the at least one pair of RNA probe sets are configured to respectively target a different portion of the duplex segment in the each of the at least one target nucleic acid sequence. As such, the sub-step of contacting both the first RNA probe set and the second RNA probe set in the each of the at least one pair of RNA probe sets with the at least one target nucleic acid sequence in the biological sample can comprise: contacting both of the first RNA probe set and the second RNA probe set in each of the at least one pair of RNA probe sets with the at least one target nucleic acid sequence in a single hybridization reaction.

According to some embodiments of the method, the first RNA probe set and the second RNA probe set in the each of the at least one pair of RNA probe sets are configured to respectively target a substantially same portion of the duplex segment in the each of the at least one target nucleic acid sequence. As such, the sub-step of contacting both the first RNA probe set and the second RNA probe set in the each of the at least one pair of RNA probe sets with the at least one target nucleic acid sequence in the biological sample can comprise: contacting one of the first RNA probe set and the second RNA probe set in each of the at least one pair of RNA probe sets with the at least one target nucleic acid sequence in a first hybridization reaction; and contacting another of the first RNA probe set and the second RNA probe set in each of the at least one pair of RNA probe sets with the at least one target nucleic acid sequence in a second hybridization reaction. Alternatively, the sub-step of contacting both the first RNA probe set and the second RNA probe set in the each of the at least one pair of RNA probe sets with the at least one target nucleic acid sequence in the biological sample can comprise: separately contacting the first RNA probe set and the second RNA probe set in each of the at least one pair of RNA probe sets with the at least one target nucleic acid sequence in a third hybridization reaction and a fourth hybridization reaction, respectively; and combining the third hybridization reaction and the fourth hybridization reaction to thereby allow a fifth hybridization reaction to proceed.

In the biological sample, one or more of the at least one target nucleic acid sequence in the biological sample are each in a polynucleotide comprising at least one un-targeted sequence. To minimize the interference of the at least one un-targeted sequence in the polynucleotide on capturing the at least one target sequence, prior to the contacting both the first RNA probe set and the second RNA probe set in the each of the at least one pair of RNA probe sets with the at least one target nucleic acid sequence in the biological sample, the method can further include: contacting at least one blocking oligo with the at least one target nucleic acid sequence such that the at least one blocking oligo respectively hybridizes with, and thereby blocks, at least one strand of each of the at least one un-targeted sequence in the polynucleotide.

Herein, if each of the one or more of the at least one target nucleic acid sequence in the biological sample is flanked by a first adaptor sequence and a second adaptor sequence in the polynucleotide, the at least one blocking oligo can be configured to respectively block one strand of the first adaptor sequence and one strand of the second adaptor sequence in the polynucleotide, and according to some embodiments, the at least one blocking oligo can be configured to respectively block both two antiparallel strands of the first adaptor sequence and both two antiparallel strands of the second adaptor sequence in the polynucleotide.

According to some embodiments, the at least one blocking oligo comprises a first blocking oligo set and a second blocking oligo set, each comprising one or more blocking oligo, configured to respectively block two antiparallel strands of one of the first adaptor sequence and the second adaptor sequence in the polynucleotide, wherein the sub-step of contacting at least one blocking oligo with the at least one target nucleic acid sequence comprises: contacting one of the first blocking oligo set and the second blocking oligo set with the at least one target nucleic acid sequence; and contacting another of the first blocking oligo set and the second blocking oligo set with the at least one target nucleic acid sequence.

According to some embodiments of the method, the step (2) of capturing each strand of the at least one target nucleic acid sequence from the biological sample can comprise:

conjugating the at least one pair of RNA probe sets on the solid support via the immobilization portion labelled onto each RNA probe in any of the first RNA probe set and the second RNA probe set in the each of the at least one pair of RNA probe sets to thereby obtain at least one pair of solid support-conjugated RNA probe sets, each pair comprising a solid support-conjugated first RNA probe set and a solid support-conjugated second RNA probe set; and

contacting both the solid support-conjugated first RNA probe set and the solid support-conjugated second RNA probe set in the each of the at least one pair of solid support-conjugated RNA probe sets with the at least one target nucleic acid sequence.

In embodiments where the solid support-conjugated first RNA probe set and the solid support-conjugated second RNA probe set may interfere with each other, such as when the first RNA probe sets and the second RNA probe set are labelled on beads that may be mixed in a common reaction, the sub-step of contacting both the solid support-conjugated first RNA probe set and the solid support-conjugated second RNA probe set in the each of the at least one pair of solid support-conjugated RNA probe sets with the at least one target nucleic acid sequence can include: contacting one of the solid support-conjugated first RNA probe set and the solid support-conjugated second RNA probe set in the each of the at least one pair of solid support-conjugated RNA probe sets with the at least one target nucleic acid sequence in a sixth hybridization reaction; and contacting another of the solid support-conjugated first RNA probe set and the solid support-conjugated second RNA probe set in the each of the at least one pair of solid support-conjugated RNA probe sets with the at least one target nucleic acid sequence in a seventh hybridization reaction. Alternatively, the sub-step of contacting both the solid support-conjugated first RNA probe set and the solid support-conjugated second RNA probe set in the each of the at least one pair of solid support-conjugated RNA probe sets with the at least one target nucleic acid sequence can include: separately contacting the solid support-conjugated first RNA probe set and the solid support-conjugated second RNA probe set in the each of the at least one pair of solid support-conjugated RNA probe sets with the at least one target nucleic acid sequence in an eighth hybridization reaction and a ninth hybridization reaction, respectively; and combining the eighth hybridization reaction and the ninth hybridization reaction to thereby allow a tenth hybridization reaction to proceed.

In embodiments where the solid support-conjugated first RNA probe set and the solid support-conjugated second RNA probe set do not interfere with each other, such as when the first RNA probe sets and the second RNA probe set are conjugated on different regions on a solid surface (e.g. a glass/plastic chip, a column or a microfluidic channel), the sub-step of contacting both the solid support-conjugated first RNA probe set and the solid support-conjugated second RNA probe set in the each of the at least one pair of solid support-conjugated RNA probe sets with the at least one target nucleic acid sequence can include: contacting both of the solid support-conjugated first RNA probe set and the solid support-conjugated second RNA probe set in the each of the at least one pair of solid support-conjugated RNA probe sets with the at least one target nucleic acid sequence in a single hybridization reaction.

According to some embodiments of the method, the immobilization portion can be configured to be able to form a stable non-covalent binding with a coupling partner conjugated onto surface of the solid support, and as such, the immobilization portion can comprise a biotin moiety, and correspondingly, the coupling partner conjugated onto surface of the solid support can comprise at least one of streptavidin, avidin, or an anti-biotin antibody. According to some other embodiments of the method, the immobilization portion can be configured to be able to form a covalent connection with a coupling partner conjugated onto surface of the solid support. The solid support can comprise at least one of a magnetic bead, a filter, a resin bead, a nanosphere, a plastic surface, a microtiter plate, a glass surface, a slide, a membrane, a microfluidic channel, a chip, or a matrix.

The method according to any one of the embodiments as described above can further include: eluting out the at least one target nucleic acid sequence from the solid support.

In the above-mentioned method and kit, the nucleic acid sequences may comprise DNA and/or RNA sequences from a test sample. The nucleic acid sequences can be natural sequence obtained from an organism, or can be artificial sequences manufactured manually. There are no limitations herein.

The test sample can be a biological sample from an organism, or can be a sample obtained artificially or obtained after appropriate handling, as long as the test sample contain one or more nucleic acid sequences. The biological sample can be DNA, RNA or any samples composed of nucleic acid sequence(s), and the samples can be chemical synthesis products or extracted from an organism, such as multicellular animals, plants, fungi, protists, bacteria, archaea, or any types of the tissue culture of the above organisms. The organism can be from any domains from prokaryota and eukaryote. The test samples may be treated prior to enrichment. For example, the nucleic acid sequences in the biological sample can be amplified prior to enrichment and testing.

The nucleic acid sequences may contain genetic markers associated with human diseases including cancer, diabetes, heart diseases, and so on, but can also include markers associated with a phenotype such as height, weight, skin color, etc. The markers may include qualitative or quantitative genetic information.

Test samples can be from any appropriate sources in the patient's body that will have nucleic acids from a cancer or lesion that can be collected and tested. Test samples can be also from any appropriate sources derived from patient tissue, such as FFPE slides, FFPE tissue blocks, and test samples can be also from any appropriate sources derived from other biological specimens, such as fossils, body remains of ancient human species or animal species. Suitable test samples may be obtained from body tissue, stool, and body fluids, such as blood, tear, saliva, sputum, bronchoalveolar lavage, urine and different organ secreted juices. In some cases, the nucleic acids will be amplified prior to testing. The samples may be collected using any means conventional in the art, including from surgical samples, from biopsy samples, from endoscopic ultrasound (EUS), phlebotomy, etc. Obtaining the samples may be performed by the same person or a different person that conducts the subsequent analysis. Samples may be stored and/or transferred after collection and before analysis. Samples may be fractionated, treated, purified, enriched, prior to assay.

According to one aspect of the invention, a portion or all nucleic acids in a sample are enriched for nucleic acid analysis. A set of probes for one or more analytes of interest is synthesized. The probes are RNA probes. The probes are transcribed from DNA template sharing the same sequences as the target nucleic acids that are going to be enriched. The RNA probes are complementary to both plus and minus strands of a target nucleic acid analyte. The RNA probes complementary to plus and minus strands of a target nucleic acid analyte are synthesized in parallel reaction systems. The set of RNA probes can be massively generated by in vitro transcription using the target nucleic acid sequences as the templates. The RNA probes are bound to a solid support. The solid support is contacted with the sample comprising nucleic acids under hybridization conditions so that complementary nucleic acids in the sample are captured on the solid support, and the solid support is washed to remove non-complementary nucleic acids. Captured nucleic acids are eluted from the solid support for further analysis.

According to another aspect of the invention, a portion or all nucleic acids in a sample are enriched for nucleic acid analysis. A set of probes for one or more analytes of interested is synthesized. The probes are RNA probes. The probes are transcribed from DNA template sharing the same sequences as the target nucleic acids that are going to be enriched. The RNA probes are complementary to both plus and minus strands of a target nucleic acid analyte. The RNA probes complementary to plus and minus strands of a target nucleic acid analyte are synthesized in parallel reaction systems. The set of RNA probes can be massively generated by in vitro transcription using the target nucleic acid sequences as the templates. The transcribed or synthesized RNA probes are sheared into fragments by sonication. Modified nucleic acids (such as biotinylated Uracil) can be incorporated into the RNA probes or additional nucleic acid modifications can happen on the ends or within nucleic acid sequences of RNA probes, either of which allows the RNA probes to be immobilized or captured and extracted after the capture procedure. RNA probes are contacted with the sample compromising nucleic acids under hybridization conditions so that complementary nucleic acids in the sample are captured. The hybridization reaction mixture is contacted with a solid support which compromises chemical structures (such as avidin or streptavidin) that specifically react with the modification groups (such as biotin) on the RNA probes. RNA probes with their captured complementary nucleic acids are immobilized, or captured, on the solid support. The solid support is washed to remove non-complementary nucleic acids. Captured nucleic acids are eluted from the solid support for further analysis.

These and other embodiments which will be apparent to those of skill in the art upon reading the specification provide the art with methods for assessing, characterizing, and detecting genetic markers, such as cancer markers, and genetic analysis, such as SNV identification. In particular, it provides methods for enriching nucleic acids for desired analytes.

Throughout the disclosure, “upstream” and “downstream” are respectively defined as nucleic acid sequences at 5′ end and 3′ end of a strand of nucleic acid sequence (DNA strand or RNA strand), unless indicated otherwise.

Unless indicated otherwise, all DNA sequences disclosed herein have a direction from a 5′ end to a 3′ end thereof.

Unless indicated otherwise, an oligo can be a single-stranded DNA oligo, or a single-stranded RNA oligo, having a sequence of at least 2 nt.

The term “about” in the disclosure generally refers to plus or minus 10% of the indicated number. For example, “about 20” may indicate a range of 18 to 22, and “about 1” may mean from 0.9-1.1. Other meanings of “about” may be apparent from the context, such as rounding off, so, for example “about 1” may also mean from 0.5 to 1.4.

The term “probe” in this disclosure is referred to as a bait molecule that can be used for capturing or enriching a target molecule, unless indicated otherwise. Specifically, in this disclosure, an RNA probe is substantially a bait RNA molecule that are used to capture and enrich a target nucleic acid sequence, including a DNA sequence or an RNA sequence or any other nucleic acid sequences that could form hybridization molecules with the RNA probes.

As used herein, the term “hybridization” or “binding” or “annealing” refers to the pairing of complementary (including partially complementary) polynucleotide strands. Hybridization and the strength of hybridization (e.g., the strength of the association between polynucleotide strands) is impacted by many factors well known in the art including the degree of complementarity between the polynucleotides, stringency of the conditions involved affected by such conditions as the concentration of salts, the melting temperature (Tm) of the formed hybrid, the temperature of the hybridization reaction, the presence of other components, the molarity of the hybridizing strands and the G:C content of the polynucleotide strands. When one polynucleotide is said to “hybridize” to another polynucleotide, it means that there is some complementarity between the two polynucleotides or that the two polynucleotides form a hybrid under high stringency conditions. When one polynucleotide is said to not hybridize to another polynucleotide, it means that there is no sequence complementarity between the two polynucleotides or that no hybrid forms between the two polynucleotides at a high stringency condition. Related, the terms “target”, “targeting”, or alike in the disclosure, such as in a phrase “an RNA probe targeting one strand of a DNA molecule” refers to the situation that the RNA probe can specifically hybridize, or bind, or anneal with the one strand of the DNA molecule.

As used herein, the term “complementary” refers to the concept of sequence complementarity between regions of two polynucleotide strands (e.g. a double-stranded structure) or between two regions of the same polynucleotide strand (e.g. a “loop” or “hairpin” structure). It is known that an adenine base of a first polynucleotide region is capable of forming specific hydrogen bonds (“base pairing”) with a base of a second polynucleotide region which is antiparallel to the first region if the base is thymine or uracil. Similarly, it is known that a cytosine base of a first polynucleotide strand is capable of base pairing with a base of a second polynucleotide strand which is antiparallel to the first strand if the base is guanine. A first region of a polynucleotide is complementary to a second region of the same or a different polynucleotide if, for example, when the two regions are arranged in an antiparallel fashion, at least one nucleotide of the first region is capable of base pairing with a base of the second region. Therefore, it is not required for two complementary polynucleotides to base pair at every nucleotide position. “Complementary” refers to a first polynucleotide that is 100% or “fully” complementary to a second polynucleotide and thus forms a base pair at every nucleotide position. “Complementary” also refers to a first polynucleotide that is not 100% complementary (e.g., 90%, or 80% or 70% complementary) contains mismatched nucleotides at one or more nucleotide positions. In one embodiment, two complementary polynucleotides are capable of hybridizing to each other under high stringency hybridization conditions.

As used herein, the term “target nucleic acid” or “target” refers to a nucleic acid containing a target nucleic acid sequence to be identified. A target nucleic acid may be single-stranded or double-stranded, and often is DNA, RNA, a derivative of DNA or RNA, or a combination thereof. A “target nucleic acid sequence,” “target sequence” or “target region” means a specific sequence comprising all or part of the sequence of a single-stranded nucleic acid. A target sequence may be within a nucleic acid template, which may be any form of single-stranded or double-stranded nucleic acid. A template may be a purified or isolated nucleic acid, or may be non-purified or non-isolated.

A target or target nucleic acid usually exists within a portion or all of a polynucleotide, and is usually a polynucleotide analyte. The identity of the target nucleotide sequence generally is known to an extent sufficient to allow preparation of various probe/bait sequences hybridizable with the target material. The target material is generally a fraction of a larger pool of molecules or it may be substantially the entire molecule such as a polynucleotide as described above.

The term “antiparallel strands” of a duplex segment of a nucleic acid sequence refers to the situation where two strands of nucleic acid sequences align in opposite directions (i.e., one strand in a 5′ end-3′ end direction, and the other in a 3′ end-5′ end direction) and form a double-stranded (i.e. “duplex”) structure due to the hybridization of the two strands. It is possible that such two antiparallel strands of the duplex segment are two complimentary DNA strands (“DNA-DNA”) of a double-stranded DNA segment, two complimentary RNA strands (“RNA-RNA”) of a double-stranded RNA segment, or two complimentary DNA and RNA strands (“DNA-RNA”) of a double-stranded DNA-RNA segment. The nucleic acid sequence can be double-stranded, and the two antiparallel strands of the duplex segment thereof are respectively the two complimentary strands of the nucleic acid sequence. Alternatively, the nucleic acid sequence can be single-stranded, where the two antiparallel strands of the duplex segment substantially forms a “hairpin” or a “loop” segment of the single-stranded nucleic acid sequence.

The term “RNA polymerase compatible with a promoter” is defined as an RNA polymerase that can recognize the promoter to thereby initiate a transcription of RNA molecules using a DNA template at a downstream of the promoter.

The terms “first”, “second”, “third”, “fourth”, “fifth”, etc., are intended to refer to a different object (i.e. component, composition, process, etc.) and indicates or suggests no actual order in the disclosure.

### BRIEF DESCRIPTION OF THE DRAWINGS

FIGS. 1A-1C respectively illustrate a flow chart of a method for enriching at least one target nucleic acid sequence from a biological sample according to two embodiments of the disclosure;

FIGS. 1D-1J respectively illustrate a schematic structure of an apparatus having each pair of RNA probe sets arranged on an inner surface thereof to allow the capture of target nucleic acid sequences from the biological sample according to several different embodiments of the disclosure;

FIG. 2A and FIG. 2B respectively illustrate a flow chart of step S100 as shown in FIG. 1A according to two embodiments of the disclosure;

FIG. 3A and FIG. 3B respectively illustrate a structural diagram of the first DNA vector and the second DNA vector for transcriptionally obtaining the first RNA molecule targeting a plus strand and the second RNA molecule targeting a minus strand of a target nucleic acid sequence according to some embodiments of the disclosure;

FIG. 3C and FIG. 3D respectively illustrate a structural diagram of the first DNA vector and the second DNA vector according to some other embodiments of the disclosure;

FIG. 4A and FIG. 4B respectively illustrate the process of preparing one pair of RNA probe sets respectively targeting a plus strand and a minus strand of one target nucleic acid sequence according to two embodiments of the disclosure;

FIG. 5A and FIG. 5B respectively illustrate a flow chart of step S200 of the method according to two embodiments of the disclosure;

FIG. 6A is a schematic diagram of the hybridization process of RNA probes with target nucleic acid sequences according to some embodiments with blocking oligos applied;

FIG. 6B illustrates a double-stranded target nucleic acid sequence consisting of a plus strand and a minus strand, and a single-stranded target nucleic acid sequence whose duplex segment having a hairpin structure, can be targeted for capturing and enrichment using the method disclosed herein;

FIGS. 7A and 7B illustrate the process of enriching target sequences from a DNA library generated for a NGS-based sequencing assay;

FIGS. 8A-8D show a performance evaluation of DNA double strand capture based on the method. (FIG. 8A) For each of the six NGS DNA libraries derived from different amounts (500 ng, 20 ng, 1 ng, 100 pg, 20 pg and 10 pg) of input genomic DNA, enrichment efficiency of 298 cancer related genes were calculated. Recovery ratios of double strand DNA capture and single strand DNA capture for all 298 genes in six libraries were quantified by real-time PCR assays detecting each gene's abundance in the libraries before and after captures with different approaches (targeting double DNA strands or targeting single DNA strand). (FIG. 8B) A plasmid was constructed with an insert sequence composed of amplicon regions of five genes whose GC contents cover a broad range (27.3% to 74.1%). (FIG. 8C) Real-time PCR analysis of sequential dilutions of the plasmid illustrated in 3E. 1, 10, 100, 1,000 and 10,000 femtomoles of the plasmids were added as template for the detection. Cₜ value for each gene obtained from each plasmid amount was plotted, and trend lines were shown. (FIG. 8D) A original genomic DNA NGS library, and the library molecules captured by RNA probes targeting a single DNA strand or both DNA strands of a target sequence region were analyzed on an agarose gel;

FIGS. 9A and 9B show SNV-calling trends and statistics of RNA probe-based DNA double strand capture WES (Whole Exome Sequencing) study;

FIGS. 10A-10C show read statistics. (FIG. 10A) Bar plot of percentage of initial reads, mapped reads and reads remained after filtering. Results were obtained from three technical replicates. Numbers of reads were shown under each bar with the unit of 1 million reads. (FIG. 10B) Stacked bar plot of subgroups of filtered reads in triple replicates. (FIG. 10C) Coverage efficiency correlation with read numbers. The percentage of target bases covered at [symbol] 10×, [symbol] 20×, [symbol] 50× and [symbol] 100× depths with 5 million to 50 million reads were shown;

FIGS. 11A and 11B show density plots of read depths to demonstrate the relationship between GC content and normalized mean read depth for (FIG. 11A) an NGS WES study using RNA probe-based DNA double strand capture approach with DNA extracted from normal human tissue; (FIG. 11B) an NGS whole genome sequencing study with DNA extracted from normal human tissue (without whole exome enrichment through any methods);

FIG. 12 shows detection of ultra-rare SNVs in libraries created from normal DNA spiked with sequentially diluted tumor DNA samples;

FIGS. 13A-13N show the 298-gene panel real-time PCR parameters;

FIG. 14 shows data yield from RNA probe-based DNA double strand capture WES sequencing; and

FIGS. 15A-15E show results of mutation and ultra-rare mutation detection by RNA probe-based DNA double strand capture NGS.

### DETAILED DESCRIPTION OF THE INVENTION

In a sequencing technology such as NGS, the enrichment of nucleic acids from diluted samples or the capture of specific nucleic acid sequences from a sample comprising a complex pool of nucleic acids is often a crucial step, but can be challenging tasks in many cases. Enrichment for desired sequences can make assays feasible that would otherwise fall below detection limits, and can improve the performance of a genetic or genomic assay.

The present disclosure provides a method for enriching nucleic acid sequences from a biological sample, which substantially utilizes RNA probes that target both of two antiparallel strands of a duplex segment of a target sequence to be enriched in a sample.

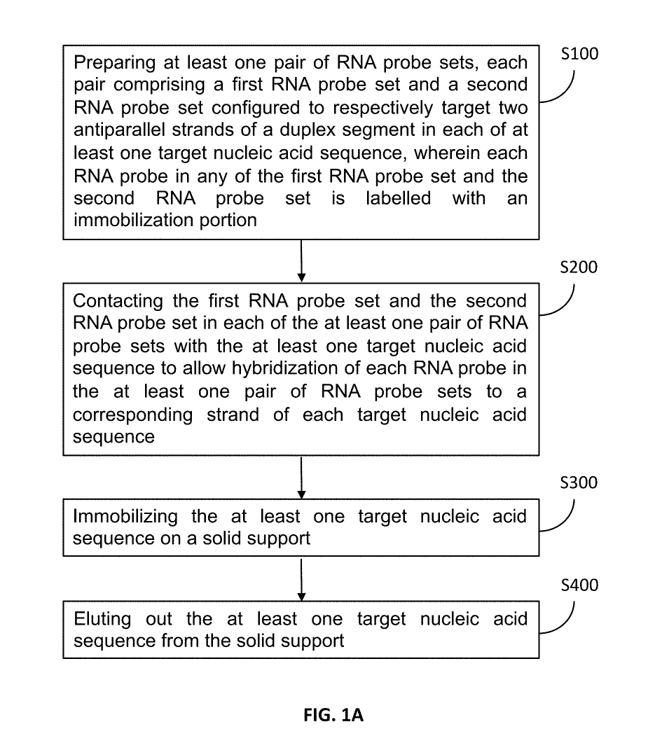

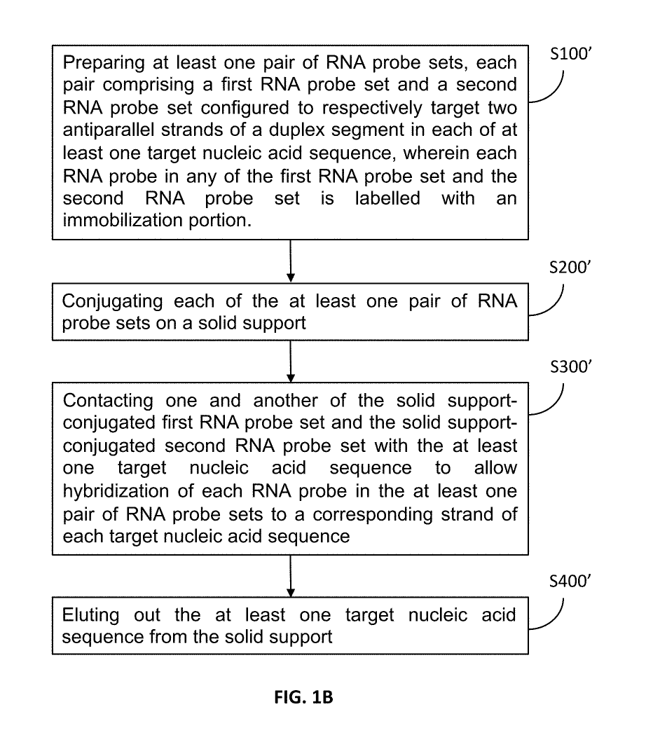

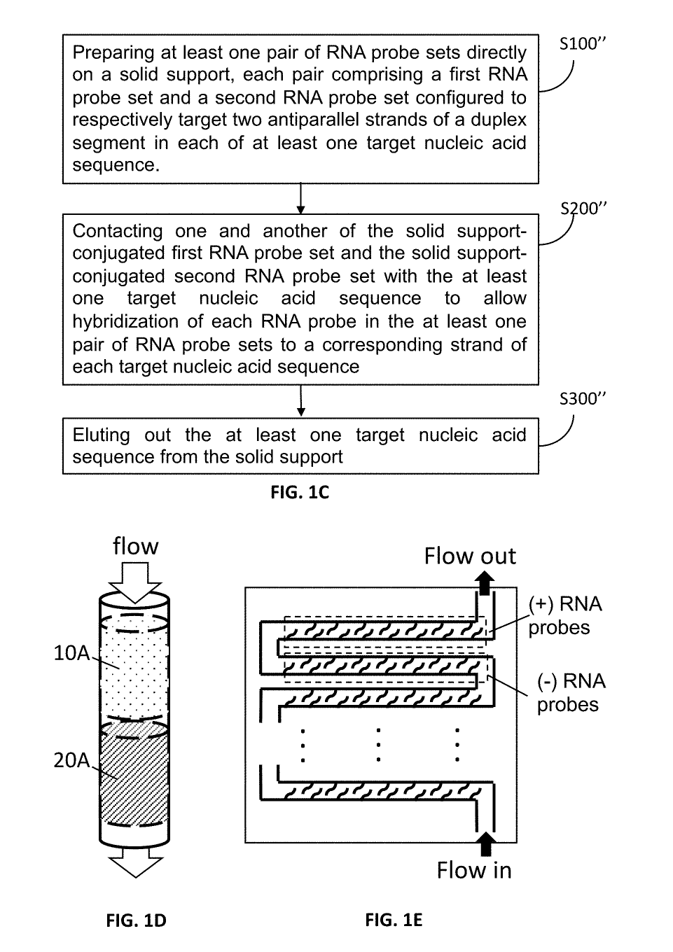

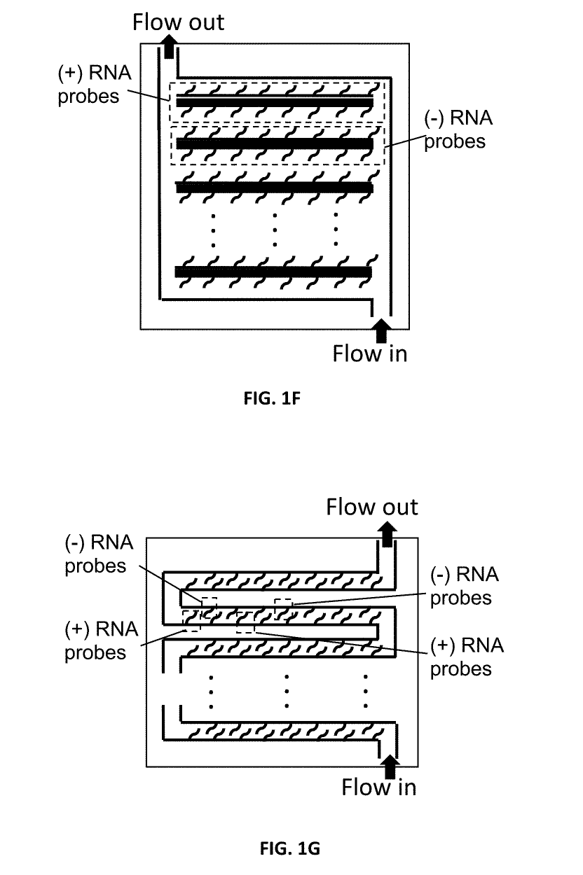

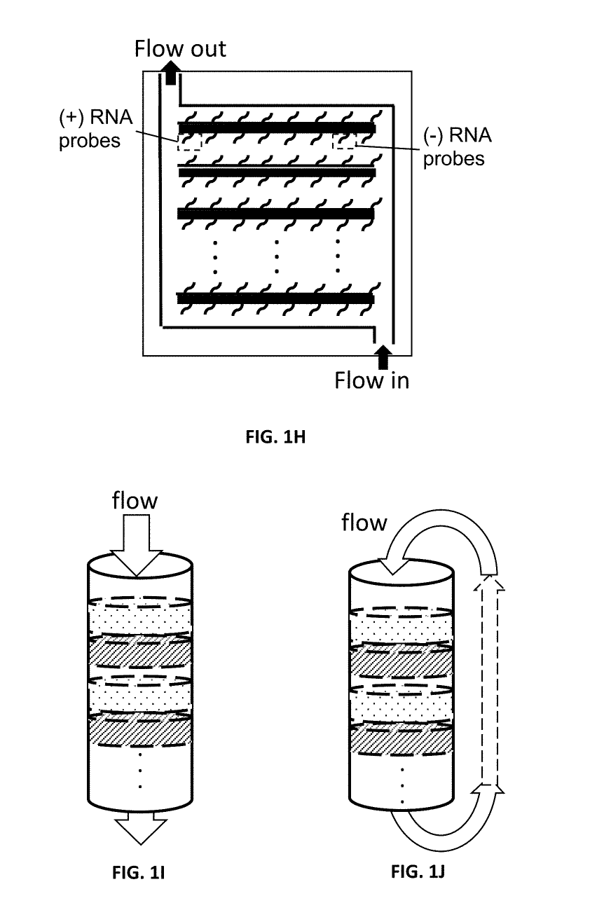

FIG. 1A illustrates a flow chart of the method for enriching at least one target nucleic acid sequence from a biological sample according to some embodiments of the disclosure. As shown in FIG. 1A, the method comprises steps as set forth in S100-S400:

S100: Preparing at least one pair of RNA probe sets, each pair comprising a first RNA probe set and a second RNA probe set configured to respectively target two antiparallel strands of a duplex segment in each of at least one target nucleic acid sequence, wherein each RNA probe in any of the first RNA probe set and the second RNA probe set is labelled with an immobilization portion.

Specifically, each pair of RNA probe sets comprises a first RNA probe set and a second RNA probe set, corresponding to one another, and each comprising one or more RNA probes. The one or more RNA probes in the first RNA probe set and the one or more RNA probes in the second RNA probe set are configured to respectively target two antiparallel strands of a duplex segment (i.e. double-stranded segment) of one of the at least one target nucleic acid sequence in the biological sample. Each RNA probe in any of the first RNA probe set and the second RNA probe set is labelled with, or comprises, an immobilization portion, configured to allow an immobilization by a solid support which will be described below in detail.

Herein, there is no limitation to the source of the biological sample, to the type of the at least one target nucleic acid sequence, or to the type of the duplex segment in each of the at least one target nucleic acid sequence.

The biological sample may be a tissue sample, from which at least one target nucleic acid sequence is obtained through a DNA or RNA purification protocol. The biological sample may be a cell-free DNA sample obtained from plasma, which contains the at least one target nucleic acid sequence in the sample. The biological sample may be derived from a treated sample, and contain, for example, a barcoded DNA library as disclosed in U.S. patent application Ser. No. 15/908,190, where each of the at least one target nucleic acid sequence contains a barcoded adaptor at one or both ends thereof. Other possibilities are possible as well.

The at least one target nucleic acid sequence in the biological sample can comprise one or more DNA molecules, one or more RNA molecules, one or more DNA-RNA hybrid molecules, or any of their combinations.

The duplex segment in each of the at least one target nucleic acid sequence may be formed by two separate DNA strands of a double-stranded DNA molecule, two separate RNA strands of a double-stranded RNA molecule, or one DNA strand and one RNA strand of a DNA-RNA hybrid molecule. The duplex segment may also be an intra-strand hairpin or alike formed within one single DNA strand or within one single RNA strand. In any of the aforementioned cases, a duplex segment substantially comprises two strand segments from one single strand (i.e. intra-strand duplex) or from two separate strands (i.e. inter-strand duplex), each having a sequence allowing a hybridization therebetween (e.g. having a sequence substantially complimentary to each other) and each running antiparallelly to each other to thereby form a double-stranded (i.e. duplex) structure.

Regardless of the type of a target nucleic acid sequence (DNA or RNA), or the type of the duplex segment therein (inter-strand or intra-strand), a first RNA probe set and a second RNA probe set can be configured to respectively target the two antiparallel strands of a duplex segment of each of the at least one target nucleic acid sequence in the biological sample, which together form a pair of RNA probe sets corresponding to the each of the at least one target nucleic acid sequence. Herein, throughout the disclosure, and also unless indicated otherwise, the two antiparallel strands of a duplex segment of each of the at least one target nucleic acid sequence in the biological sample are termed a plus strand and a minus strand of the each of the at least one target nucleic acid sequence.

In other words, the at least one RNA probe in the first RNA probe set and the at least one RNA probe in the second RNA probe set are respectively configured to target the plus strand and the minus strand, or alternatively the minus strand and the plus strand, of one of the at least one target nucleic acid sequence. By means of the at least one pair of RNA probe sets where a first RNA probe set and a second RNA probe set in each pair respectively target two antiparallel strands of a duplex segment in each target nucleic acid sequence, each strand of the at least one target nucleic acid sequence can be captured or enriched from the biological sample. For a typical example, each target nucleic acid sequence can be a double-stranded DNA molecule, which has a plus strand and a minus strand that runs antiparallelly to form a duplex. By means of the at least one pair of RNA probe sets where a first RNA probe set and a second RNA probe set in each pair respectively target both of the two antiparallel strands (i.e. the plus strand and the minus strand) of each target nucleic acid sequence, each strand, including both the plus strand and the minus strand, of each target DNA molecule, can be captured or enriched from the biological sample.

This notably contrasts with a conventional approach where only one strand of each target nucleic acid sequence is captured since only one RNA probe or one RNA probe set targeting only one strand of each target nucleic acid sequence is utilized. More specifically, in conventional approach a valid capture is typically a 1^{st} degree reaction between two complementary sequences that are respectively from a target nucleic acid sequence and a probe. However, in this present disclosure, a valid capture is a 2^{nd} degree reaction between a target nucleic acid molecule having a duplex segment (e.g. a duplex double-stranded DNA molecule) and each of a pair of RNA probes (i.e. a first RNA probe and a second RNA probe) that respectively target the two antiparallel strands of the duplex segment, where the hybridization of one strand in the duplex segment of the target nucleic acid molecule with one of the pair of RNA probes can help expose the other strand of the duplex segment to thereby facilitate the hybridization of the other of the pair of RNA probes therewith. Therefore a higher capture efficiency can be realized, and such an effect has been observed in the experiment as detailed below.

Herein, the solid support can comprise at least one of a magnetic bead, a microfluidic channel, a filter, a resin bead, a nanosphere, a plastic surface, a microtiter plate, a glass surface, a slide, a membrane, a microfluidic channel, a chip, or a matrix, which is labelled, conjugated, or attached, with the coupling partner corresponding to the immobilization portion. The solid support can be part of an apparatus, such as a chip, a column, a tube, or a channel (such as a microfluidic channel in a microfluidic chip).

The immobilization portion labelled on each RNA probe can include a biotin moiety, and correspondingly the solid support can comprise comprises at least one of a magnetic bead, a filter, a resin bead, a nanosphere, a plastic surface, a microtiter plate, a glass surface, a slide, a membrane, a microfluidic channel, a chip, or a matrix, and the solid support can be labelled with at least one of streptavidin, avidin, or an anti-biotin antibody. As such, the RNA probes labelled with, or carrying, the biotin moiety can form a secure non-covalent binding with the solid support conjugated with a biotin-coupling partner, such as streptavidin, avidin, or an anti-biotin antibody, which facilitates the capture of target nucleic acid sequences hybridized by the RNA probes. Other examples of the immobilization portion-coupling partner pair can include, but is not limited to, a carbohydrate-lectin pair, an antigen-antibody pair and a negative charged group-positive charged group static interacting pair.

In addition to the above embodiments where each RNA probe binds to the solid support through a non-covalent binding between the immobilization portion and the coupling partner pair, the secure coupling between each RNA probe and the solid support can be via a covalent connection (or cross-linking). As such, the immobilization portion and the coupling partner can respectively be one and another of a cross-linking pair. Examples of the cross-linking pair include an NHS ester-primary amine pair, a sulfhydryl-reactive chemical group pair (e.g. cysteines, or other sulfhydryls such as maleimides, haloacetyls, and pyridyl disulfides), an oxidized sugar-hydrazide pair, photoactivatable nitrophenyl azide's UV triggered addition reaction with double bonds leading to insertion into C—H and N—H sites or subsequent ring expansion to react with a nucleophile (e.g., primary amines), or carbodiimide activated carboxyl groups to amino groups (primary amines), etc. It is noted that the RNA probes can be conjugated on the solid support after synthesis, or can be synthesized directly on the solid support. Any method that could result in RNA probes to be linked on a solid support can be adopted. There is no limitation herein.

Herein it is noted that the first RNA probe set and the second RNA probe set in each of the at least one pair of RNA probe sets can be configured to target a same portion, or a distinct portion, of the duplex segment in one of at least one target nucleic acid sequence.

According to some embodiments, a first RNA probe set and a second RNA probe set in a corresponding pair of RNA probe set are configured to target a same portion of the duplex segment of one target nucleic acid sequence, and as such, the RNA probe(s) in the first RNA probe set and the RNA probe(s) in the second RNA probe set have substantially complimentary sequences. As such, in certain embodiments where the RNA probes between the first RNA probe set and the second RNA probe set could potentially interact, or interfere, with one another in a single hybridization due to the unwanted hybridization therebetween, such as in cases where the RNA probes are labelled onto beads or matrix, contacting of the biological sample containing target nucleic acid sequences by the first RNA probe set and the second RNA probe set is preferably performed in a sequential manner. This can be realized by sequentially contacting the biological sample containing target nucleic acid sequences with magnetic beads respectively carrying one and another of the first RNA probe set and the second RNA probe set, which will be described in detail below. This can also be realized by allowing the biological sample to flow sequentially through two different layers (10A and 20A) of a column which comprise a matrix conjugated respectively with the first RNA probe set and the second RNA probe set, as illustrated in FIG. 1D.

It is noted, however, in some other embodiments where the RNA probes are separately conjugated onto a solid support which has no or little interactions due to, for example, positional compartmentation on the surface of the solid support, such as in cases where RNA probes from the first RNA probe set and RNA probes from the second RNA probe set are separately conjugated onto different regions of a microfluidic channel (FIG. 1E) or different regions of a chip (FIG. 1F), or are mixedly arranged on a common region of a microfluidic channel (FIG. 1G) or a chip (FIG. 1H) with a relatively large distance between each RNA probe from the first RNA probe set and each RNA probe from the second RNA probe set (thereby substantially preventing an interaction therebetween), there is no need for a sequential contact between the biological sample with the first RNA probe set and the second RNA probe set, and the biological sample can be applied to contact both the first RNA probe set and the second RNA probe set simultaneously. Yet depending on certain practical needs, the biological sample can still be allowed to sequentially contact RNA probes in the first RNA probe set and the second RNA probe set by flowing sequentially through two different regions of a microfluidic channel or a chip corresponding respectively to the first RNA probe set and the second RNA probe set as illustrated in FIG. 1E and FIG. 1F.

It is further noted that the employment of a column or a microfluidic channel further provides a convenience for repeated contacts of the biological sample with RNA probes, either by arranging the first RNA probe set and the second RNA probe set in series (as illustrated in FIG. 1I), by arranging the biological sample to recirculate (as illustrated in FIG. 1J), or by combination of these two approaches. The details for each of these above different embodiments of the method for capturing target nucleic acid sequences from the biological sample by means of the at least one pair of RNA probe sets will be provided below.

As such, the above FIGS. 1D-1J respectively illustrate several different embodiments where each pair of RNA probe sets arranged on an inner surface of an apparatus (microfluidic channel or a chip) to allow the capture of target nucleic acid sequences from the biological sample. It is noted that these embodiments are illustrating only, and there can be other apparatuses as well.

According to some other embodiments, a first RNA probe set and a second RNA probe set in a corresponding pair of RNA probe set are configured to target a different portion of the duplex segment of one target nucleic acid sequence. In other words, a first RNA probe set and a second RNA probe set in a corresponding pair of RNA probe set are respectively configured to target a first portion in a first strand and a second portion in a second strand of the one target nucleic acid sequence, wherein the first strand and the second strand are the two antiparallel strands of the duplex segment, and the first portion and the second portion are two different portions of the duplex segment of the one target nucleic acid sequence. As such, the RNA probe(s) in the first RNA probe set and the RNA probe(s) in the second RNA probe set have substantially no complimentary sequences. As such, the biological sample containing target nucleic acid sequences can be applied to contact both the first RNA probe set and the second RNA probe set simultaneously, regardless of what type of solid support is utilized for coupling with the RNA probes.

In order to prepare the at least one pair of RNA probe sets in step S100, any one of the first RNA probe set and the second RNA probe set in each pair can be prepared by a manner of direct chemical synthesis or by a manner of transcription, or by a various combination of these approaches, and can be labelled with the immobilization portion during or after the synthesis/transcription process. These different embodiments will be described below in detail.

According to some embodiments, each RNA probe in any of the first RNA probe set and the second RNA probe set corresponding to each of the at least one pair of RNA probes can be synthesized directly by chemical reactions (i.e. the manner of direct chemical synthesis). Depending on different situations, the immobilization portion can be labelled to each RNA probe during the synthesis process or after the synthesis process, or each RNA probe can be directly synthesized from a solid support that is covalently connected with one end of each RNA probe through the immobilization portion.

According to some other embodiments, each RNA probe in any of the first RNA probe set and the second RNA probe set for each pair of the RNA probe sets can be respectively and separately obtained through a transcription reaction (i.e. the manner of transcription) over a pair of DNA vectors corresponding thereto, and the immobilization portion can be labelled to each RNA probe during the transcription process or after the transcription process.

Regarding the transcription process, each of the pair of DNA vectors can comprise a DNA template and a promoter (i.e. transcription promoter). The DNA template can comprise a sequence whose transcription gives rise to an RNA molecule corresponding to either of the two antiparallel strands of the duplex segment of the one target nucleic acid sequence. The promoter is configured to be recognized by an RNA polymerase to thereby allow the transcription reaction to occur, and can be at an upstream of, a downstream of, or within a target DNA sequence in the DNA vector.

Compared with the manner of direct chemical synthesis, this transcription-based approach to obtain RNA probes is relatively more cost-effective, and allows for the production of a relatively larger amount of the RNA probes.

In the aforementioned embodiments where the at least one RNA probe in each of the at least one pair of RNA probe sets is prepared by transcription, step S100 can comprise:

S110: preparing a plurality of DNA vectors, comprising at least one pair of DNA vectors, each pair comprising a first DNA vector and a second DNA vector configured to respectively allow a separate transcription of a first RNA molecule and a second RNA molecule targeting respectively two antiparallel strands of a duplex segment of each of the at least one target nucleic acid sequence.

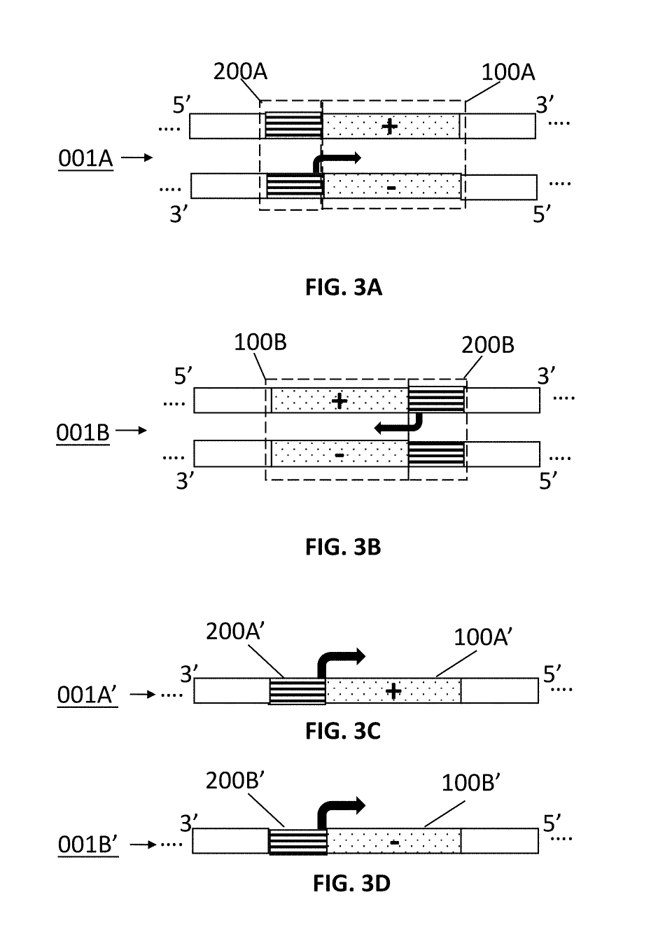

According to some embodiments as illustrated in FIG. 3A and FIG. 3B, the first DNA vector 001A and the second DNA vector 001B in each of the at least one pair of DNA vectors can respectively comprise a first DNA template 100A at a downstream of a first transcription promoter 200A and a second DNA template 100B at a downstream of a second transcription promoter 200B. Each of the first DNA template 100A and the second DNA template 100B is substantially a double-stranded DNA segment (indicated by the box with dotted lines in the two figures), configured to allow transcription of RNA molecules using one strand thereof as a template under action of the transcription promoter in a transcription reaction.

Specifically, the first DNA vector 001A comprises a first transcription promoter 200A at a 5′ end (i.e. upstream) of a strand of the double-stranded first DNA template 100A that corresponds to a plus strand of the one target nucleic acid sequence (as indicated by the “+” sign), and thus is configured to allow the transcription of a first RNA molecule complementary to a minus strand of the one target nucleic acid sequence (as indicated by the “−” sign) as a transcription template. As such, the RNA molecules produced by the first DNA vector 001A can specifically target (i.e. hybridize or bind or anneal with) the minus strand of the one target nucleic acid sequence.

The second DNA vector 001B comprises a second transcription promoter 200B at a 5′ end (i.e. upstream) of a strand of the double-stranded first DNA template 100B that corresponds to a minus strand of the one target nucleic acid sequence (as indicated by the “−” sign), and thus is configured to allow the transcription of a second RNA molecule complementary to a plus strand of the one target nucleic acid sequence (as indicated by the “+” sign) as a transcription template. As such, the RNA molecules produced by the second DNA vector 001B can specifically target (i.e. hybridize or bind or anneal with) the plus strand of the one target nucleic acid sequence.

As such, in each corresponding pair of DNA vectors, the first DNA vector 001A and the second DNA vector 001b respectively allow transcription of a first RNA molecule that specifically target the minus strand and a second RNA molecule that specifically target the plus strand of the one target nucleic acid sequence.

In each of the at least one pair of DNA vectors (i.e. the first DNA vector and the second DNA vector) as described above, the first promoter 200A and the second promoter 200B can be substantially same or different, and different pairs of DNA vectors can have same or different promoters. These promoters can include a T3 promoter, a T7 promoter, a SP6 promoter, or a species-specific or tissue-specific promoter. Herein, the double-stranded DNA segment in each of the first DNA vector and the second DNA vector that correspond to each target nucleic acid sequence in the sample can comprise a genomic DNA fragment, a gene coding sequence (CDS) or such sequences in an existing construct (such as commercially available gene expression constructs), or can be derived from reverse-transcription of an RNA sequence, such as an mRNA sequence, or can comprise segments that are artificially synthesized or assembled.

It is noted that the above embodiments as shown in FIG. 3A and FIG. 3B, where each of the first DNA vector 001A and the second DNA vector 001B in each pair of DNA vectors is substantially a double-stranded DNA vector, and each of the first DNA template 100A and the second DNA template 100B exists as a double-stranded DNA segment and is at a downstream of a transcription promoter, serves as an illustrating example only and does not impose a limitation to the scope of the present disclosure. Other embodiments are also possible.

For example, according to some other embodiments as illustrated in FIG. 3C and FIG. 3D, each of the first DNA vector 001A′ and the second DNA vector 001B′ can be a single-stranded DNA vector (such as a phagemid or phasmid, or a vector containing a cDNA molecule produced from a reverse-transcription reaction from an RNA sequence). The first DNA vector 001A′ comprises a first promoter 200A′ at a 3′ end of a first DNA template 100A′ which corresponds to a plus strand of a duplex segment of one target nucleic acid sequence (as indicated by the “+” sign). The second DNA vector 001B′ comprises a second promoter 200B′ at a 3′ end of a second DNA template 100A′ which corresponds to a minus strand of the duplex segment of the one target nucleic acid sequence (as indicated by the “−” sign). Thus transcription of the first DNA vector 001A′ and the second DNA vector 001B′ can respectively produce RNA molecules that target the plus strand and the minus strand of the duplex segment of the one target nucleic acid sequence.

According to yet some other embodiments of the disclosure, the first DNA vector and the second DNA vector in each pair of DNA vectors can be of a different type. For example, the first DNA vector can be a double-stranded DNA vector whereas the second DNA vector can be a single-stranded DNA vector, and it is further configured such that transcription of the first DNA vector and the second DNA vector can respectively produce RNA molecules that target the two antiparallel strands (e.g. plus strand/minus strand or minus strand/plus strand) of the duplex segment of the one target nucleic acid sequence.

In any of the above embodiments of the DNA vectors, a transcription promoter is disposed at an upstream of a target DNA sequence. The relative position of the transcription promoter is not limited to the upstream of a target DNA sequence, and can be within, or at a downstream of a target DNA sequence as well, depending on specific cases.

As such, there is no limitation to the nature and type of the first DNA vector and the second DNA vector in each pair of DNA vectors, as long as the respective transcription reaction over the first DNA vector and the second DNA vector can give rise to the first RNA molecule and the second RNA molecule targeting respectively two antiparallel strands of a duplex segment of each target nucleic acid sequence.

After sub-step S110, there are several embodiments of step 100 of the method where the first RNA probe set and the second RNA probe set in each of at least one pair of RNA probe sets are obtained by transcription reactions over the first DNA vector and the second DNA vector in each of the plurality of DNA vectors, and each probe in any of the first RNA probe set and the second RNA probe set is labelled with, or carries, the immobilization portion.

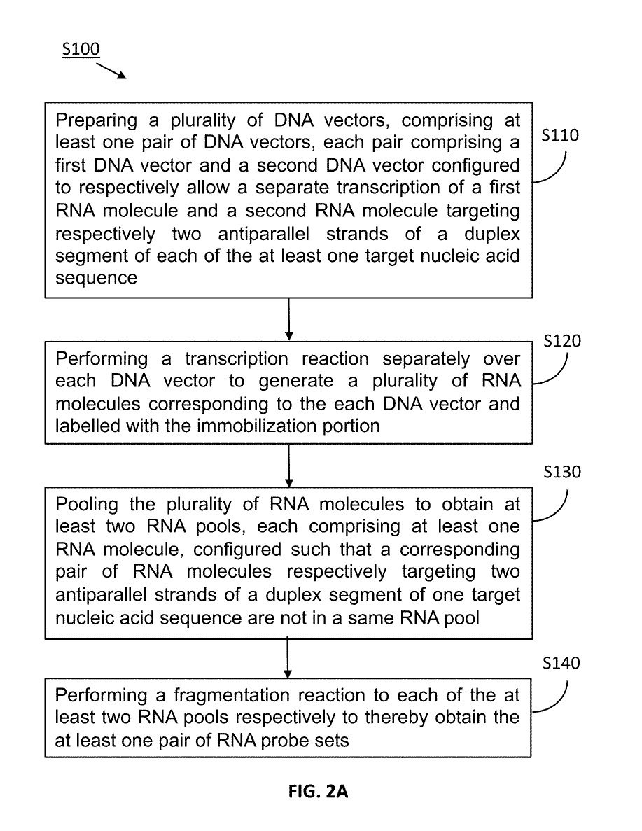

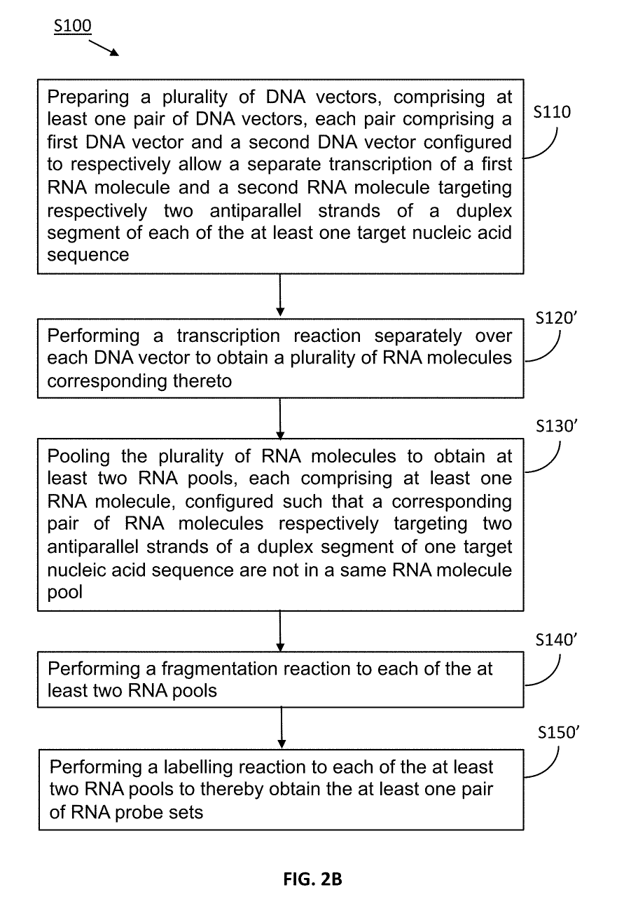

According to some preferred embodiments as illustrated in FIG. 2A, the immobilization portion is labelled during transcription process. As such, after sub-step S110, step S100 of the method further comprises:

S120: performing a transcription reaction separately over each DNA vector to generate a plurality of RNA molecules corresponding to the each DNA vector and labelled with the immobilization portion.

Herein the transcription reaction can be a regular in vitro transcription reaction, and involves an RNA polymerase and four nucleoside triphosphates (ATP, UTP, GTP, CTP, collectively as NTPs). The RNA polymerase can recognize a transcription promoter (i.e. the first DNA transcription promoter or the second DNA transcription promoter) of a DNA template (i.e. the first DNA template or the second DNA template corresponding respectively to the two antiparallel strands of each target nucleic acid sequence), to thereby allow the transcription of RNA molecules having sequences targeting/complementary to the plus strand/minus strand of each nucleic acid sequences (i.e. the first RNA molecule and the second RNA molecule). The RNA polymerase can be any enzyme that triggers DNA-dependent RNA polymerization, and can be, for example a T7 RNA polymerase.

The RNA molecules can be purified from the reaction using an RNA purification protocol, and the DNA molecules in the reaction can be eliminated by applying enzymes that can degrade DNA molecules, such as DNase. Such enzymes need to be completely removed from the RNA molecules if the targeted nucleic acid sequences being captured include DNA molecules, but this removal step can be skipped if the targeted nucleic acid sequences are nucleic acids that are not susceptible to DNase-induced damage, such as RNA molecules. Other approaches to remove DNA molecules or to separate RNA molecules from the DNA molecules are also possible. For example, the DNA vectors are pre-immobilized to solid support that could be readily removed from the reaction after the transcription reaction is finished without removing the transcribed RNA.

The RNA molecules can be directly extracted from the system through applying the interaction between the coupling pairs, such as using streptavidin beads to extract RNA molecules that are biotinylated. The transcription reaction can also be an in vivo transcription reaction, and can be synthesized in an organism, such as a bacterium (e.g. E. coli), a fungus (e.g. yeast), a mammalian cell line, etc. The RNA molecules can be extracted based on a regular RNA extraction protocol, and can be left in the system to perform real-time labeling or capturing of its target nucleic acid sequences.

Herein the sub-step S120 can directly generate a plurality of RNA molecules, each labelled with the immobilization portion. Specifically, the immobilization portion labelled on each of the RNA molecules, which is further labelled on the RNA probes derived from the RNA molecules, can facilitate the immobilization (or capturing) of the at least one target nucleic acid sequence on a solid support in step S300 (see below), due to a stable coupling between the immobilization portion and the solid support.

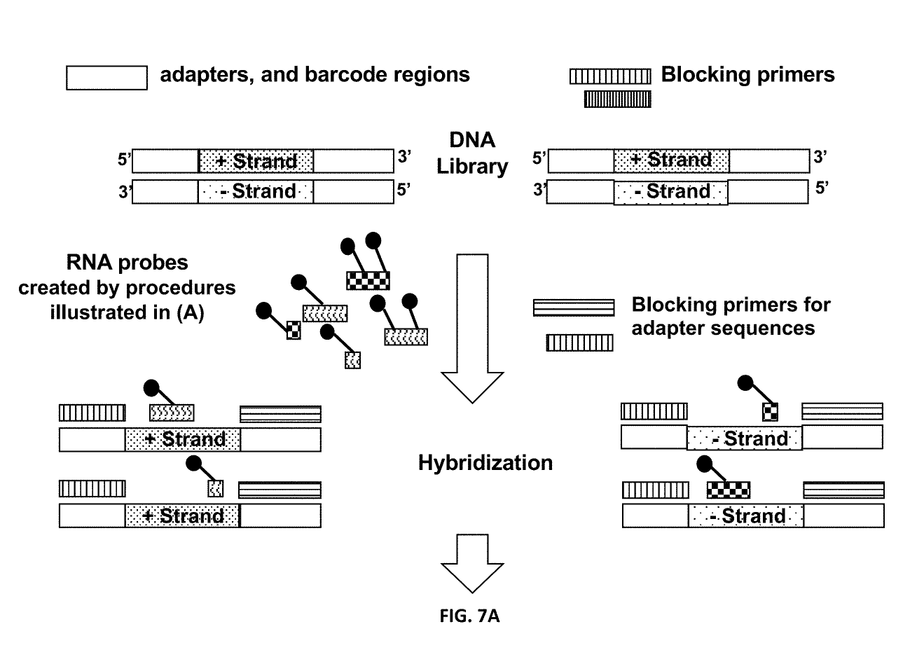

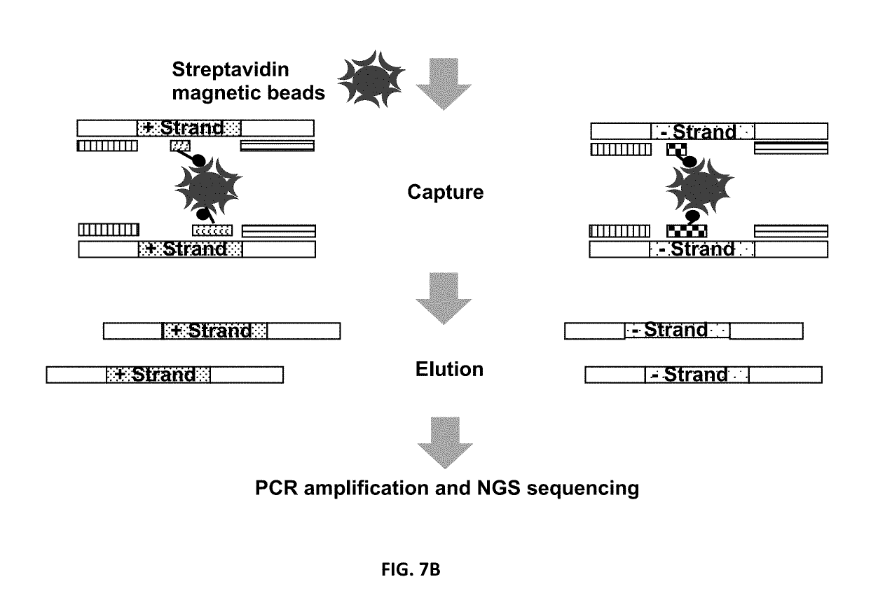

The stable coupling can be mediated by a secure and stable non-covalent binding, or by a covalent connection (i.e. cross-linking) between the immobilization portion and a corresponding coupling partner conjugated onto the solid support. Depending on different types of a solid support, and different types of coupling between each RNA probe labelled with the immobilization portion and the solid support conjugated with the coupling partner, there can be different embodiments. For example, in some preferred embodiments, the immobilization portion is a biotin moiety, and the coupling partner can be a streptavidin, avidin, or an anti-biotin antibody, which is attached onto, or conjugated with a solid support such as magnetic beads, as illustrated in FIG. 7A and FIG. 7B. In another example, each RNA probe is conjugated onto an inner surface of a microfluidic channel or a chip via a covalent connection, as illustrated in FIGS. 1E-1H.

As such, through the stable coupling between each immobilization portion on each RNA probe that derives from the RNA molecules obtained thereby and the coupling partner corresponding thereto that is conjugated onto the solid support, the at least one RNA probe can be immobilized by the solid support, in turn facilitating subsequent enrichment, isolation, and purification of target nucleic acid sequences.

It is noted that in order to introduce convenience for other applications (e.g. visualization), each RNA probe can be further labelled with other functional portion(s) in addition to the immobilization portion. Examples include a dye, a fluorophore group, or a chemical group, etc.

In some specific embodiments that are preferred, the transcription reaction in sub-step S120 can include addition of a mix of UTPs comprising biotin-labelled UTPs (i.e. herein the biotin moiety serves as an immobilization portion in the at least one functional portion) and non-biotin-labelled UTPs, wherein the biotin-labelled UTPs have a relative molar percentage of ˜2%-100% of the total UTPs. In other words, among all the UTPs added in the reaction, the biotin-labelled UTPs can take a molar percentage between about 2% and about 100% (i.e. all the UTPs added are biotin-labelled UTPs). As such, after transcription, each of the plurality of RNA molecules with a length of about 200 nt can be labelled with biotin in some or all of its U residue.

S130: pooling the plurality of RNA molecules to obtain at least two RNA pools, each comprising at least one RNA molecule, configured such that a corresponding pair of RNA molecules respectively targeting two antiparallel strands of a duplex segment of one target nucleic acid sequence are not in a same RNA pool.

After transcription in sub-step S120, the plurality of labelled RNA molecules can be pooled into at least two RNA pools (or called RNA libraries), each comprising at least one RNA molecule. It is configured that a corresponding pair of RNA molecules (i.e. the first RNA molecule and the second RNA molecule that respectively targets two antiparallel strands of a duplex segment of one target nucleic acid sequence) are not in a same RNA pool to thereby avoid an interference in subsequent steps of hybridization and enrichment of target nucleic acid sequences.

In one specific embodiment, a plurality of RNA molecules are pooled into two RNA pools (a first RNA pool and a second RNA pool), and the first RNA pool and the second RNA pool respectively includes RNA molecules that each specifically target one, but not both, of the plus strand and the minus strand of each target nucleic acid sequence.

It is noted that the sub-step S130 can be modified depending on different needs. For example, when doing pooling, each of the plurality of RNA molecules can have a same ratio, or can have a different ratio in order to ensure a highly efficient capture/enrichment of the different sequence fragments of the at least one target nucleic acid sequences in the biological sample. For example, in cases where some specific target nucleic acid sequences are difficult to capture (which can be based on previous knowledge, or can be known by a preliminary experiment applying all of the steps S100-S400, and identifying some target nucleic acid sequences that are unsatisfactorily captured), the abundance for the pair of RNA probe sets, or the abundance for the RNA probes targeting one specific strand corresponding to these specific target nucleic acid sequences can be increased (e.g. by ˜10 fold or higher, or by ˜1.5 fold; there is no limitation herein) in the sub-step S130 to thereby increase the efficiency for capture.

It is further noted that in addition to the above optimization (i.e. adjustment of ratios) in sub-step S130, a different segment in one target sequence can be selected for generation RNA probes in order for an optimized capture if RNA probes generated from one particular target segment are not able to offer expected capturing efficiency.

S140: performing a fragmentation reaction to each of the at least two RNA pools respectively to thereby obtain the at least one pair of RNA probe sets.

In order to facilitate the efficiency in the subsequent steps S200 and S300, the relatively long RNA molecules in each RNA pool can be preferably fragmented into relatively shorter fragments of ˜100-150 nt, which can be done, for example, by enzymatic reactions or sonication. Conditions for the enzymatic reactions or sonication reactions for nucleic acids are well-known in the field and can be used as is appropriate and convenient. After fragmentation, the at least one RNA probe in each of the at least two RNA probe pools can have a length of at least 2 nt, preferably 100-150 nt.

Herein it is noted that it is possible to reverse the order of sub-steps S130 and S140. For example, after sub-step S120 to obtain the plurality of RNA molecules labelled with at least one functional portion, the plurality of RNA molecules can be fragmented (i.e. S140) before pooling (i.e. S130) to thereby obtain the at least one pair of RNA probe sets.

Besides the embodiments as mentioned above, according to some other embodiments of the disclosure, a plurality of RNA molecules can alternatively be transcribed without labeling, and the at least one functional portion can be labelled thereafter. FIG. 2B illustrates one embodiment of step S100. As shown in the figure, after sub-step S110, step 100 further comprises:

S120′: performing a transcription reaction separately over each DNA vector to obtain a plurality of RNA molecules corresponding thereto.

Herein the sub-step S120′ is substantially identical to the aforementioned sub-step S120, except that no functional portion-labelled NTPs (e.g. biotin-labelled UTP), is added to the transcription reaction.

S130′: pooling the plurality of RNA molecules to obtain at least two RNA pools, each comprising at least one RNA molecule, configured such that a corresponding pair of RNA molecules respectively targeting two antiparallel strands of a duplex segment of one target nucleic acid sequence are not in a same RNA molecule pool.

Herein the sub-step S130′ is substantially identical to the aforementioned sub-step S130, and the technical details are thus skipped herein.

S140′: performing a fragmentation reaction to each of the at least two RNA pools.

Herein the sub-step S140′ is substantially identical to the aforementioned sub-step S140, and the technical details are thus skipped herein.

S150′: performing a labelling reaction to each of the at least two RNA pools to thereby obtain the at least one pair of RNA probe sets.

Herein through S150′, the immobilization portion can be labelled onto each RNA probe in any of the at least two RNA pools. According to some preferred embodiment, the immobilization portion is a biotin moiety, and can be labelled at a 5′ end, and/or a 3′ end, and/or an intra-strand nucleic acid residue of each RNA probe in this sub-step. It is noted that other functional portion(s) may also be labelled onto each RNA probe in any of the at least two RNA pools. The technical details have been provided above and are skipped herein.

Herein it is noted that it is possible to alter the order of sub-steps S130′-S150′. For example, after sub-step S120′ to obtain the plurality of RNA molecules, the plurality of RNA molecules can be fragmented (i.e. S140′) and labelled (i.e. S150′) before pooling (i.e. S130) to thereby obtain the at least one pair of RNA probe sets. Alternatively, after sub-step S120′, the plurality of RNA molecules can be labelled (i.e. S150′) before fragmentation (i.e. S140′) and pooling (i.e. S130) to thereby obtain the at least one pair of RNA probe sets. There are no limitations herein.

In the following, two specific embodiments are provided to respectively illustrate two different processes of preparing one pair of RNA probe sets (i.e. a first RNA probe set and a second RNA probe set), which respectively target the two antiparallel strands (i.e. the plus strand and the minus strand) of a double-stranded segment of one target nucleic acid sequence.

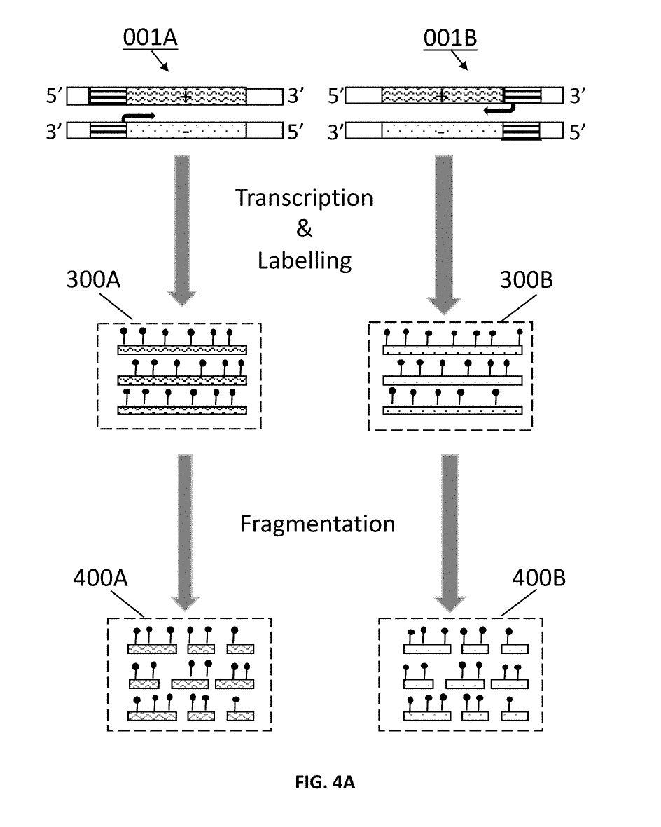

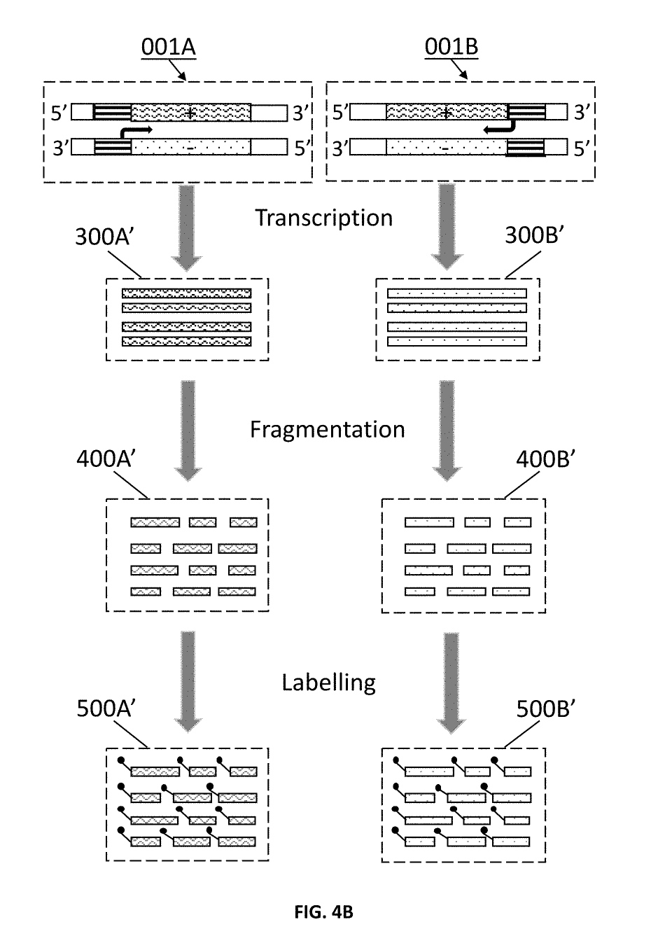

In one embodiment as illustrated in FIG. 4A, the pair of RNA probe sets are prepared and biotin-labelled during a same transcription and labelling process. Specifically, the two transcription reactions are respectively performed over the first DNA vector 001A and the second DNA vector 001B as shown in FIGS. 3A and 3B to thereby separately generate a plurality of first RNA molecules 300A and a plurality of second RNA molecules 300B, configured to respectively target the minus strand and the plus strand of the one target nucleic acid sequence to be enriched or captured. At the same time, the plurality of first RNA molecules 300A and the plurality of second RNA molecules 300A′ are each also labelled with one or more biotin moieties (shown as a filled dot linked with each RNA probe in FIG. 4A). This can be done by providing to the transcription reaction a mixture of UTPs comprising biotinylated UTPs (i.e. biotin-labelled UTPs) and regular UTPs (i.e. non-biotin-labelled UTPs), wherein the biotin-labelled UTPs can have a relative molar percentage of ˜2%-100% of the total amount of UTPs.

In one specific example, a mixture of biotin-labelled UTPs and regular UTPs having a ratio of ⅓ (i.e. the biotin-labelled UTPs have a relative molar percentage of 25% of the total amount of UTPs) can be added to the transcription reaction. As such, about ¼ of U residues in the whole RNA sequence can be found to be labelled with a biotin moiety. Then after the process of transcription and labelling, the plurality of first RNA molecules 300A and the plurality of second RNA molecules 300A′ are separately fragmented into the first RNA probe set 500A and the second RNA probe set 500B, which can preferably be ˜100-150 nt in length.

In another embodiment as illustrated in FIG. 4B, the pair of RNA probe sets are biotin-labelled after the transcription process. Specifically, the two transcription reactions can be respectively performed over the first DNA vector 001A the second DNA vector 001B as shown in FIGS. 3A and 3B to thereby separately generate a plurality of first RNA molecules 300A′ and a plurality of second RNA molecules 300B′, configured to respectively target the minus strand and the plus strand of the one target nucleic acid sequence to be enriched or to be captured. After transcription, the plurality of first RNA molecules 300A′ and second RNA molecules 300B′ are separately fragmented into the first RNA probe set 400A′ and the second RNA probe set 400B′, which are then respectively labelled with the biotin moiety (shown as a filled dot linked with one end of each RNA probe) to thereby obtain biotin-labelled first RNA probe set 500A′ and second RNA probe set 500B′.

Herein labelling of the biotin moiety at a 5′ end of each RNA probe is for illustrating purpose only, which can be at a 3′-end or an intra-strand segment thereof as well. According to some specific embodiment, in each of the ligation reactions, a 5′ phosphate terminus of a biotin-labeled nucleotide can be ligated to a 3′ hydroxyl terminus of each RNA probe in each of the at least one pair of RNA probe sets, which can be catalyzed by means of an RNA ligase. The RNA ligase can comprise at least one of T4 RNA ligase or CircLigase RNA ligase.

According to some other embodiment, the immobilization portion can be labelled in an intra-strand segment of an RNA probe, in a form of an RNA adduct (or a chemical addon) by means of a technology known to the field, whose description is skipped herein.

As such, after the above mentioned sub-steps S110-S140 (illustrated in FIG. 2A) or sub-steps S110-S150′ (illustrated in FIG. 2B), at least two RNA probe pools are generated, each comprising at least one immobilization portion-labelled RNA probe. Together, the at least two RNA probe pools substantially includes at least one pair of RNA probe sets, each pair comprising a first labelled RNA probe set and a second labelled RNA probe set, respectively targeting two antiparallel strands of a duplex segment of one of the at least one target nucleic acid sequence.

After preparation of the at least one pair of RNA probe sets in step S100, the at least one pair of RNA probe sets can be used to contact with target nucleic acid sequences for hybridization before capture and enrichment of the at least one target nucleic acid sequence using the at least one pair of RNA probe sets as baits.

S200: Contacting the first RNA probe set and the second RNA probe set in each of the at least one pair of RNA probe sets with the at least one target nucleic acid sequence to allow hybridization of each RNA probe in the at least one pair of RNA probe sets to a corresponding strand of each target nucleic acid sequence.

Depending on whether the first RNA probe set and the second RNA probe set corresponding to a pair of RNA probe sets interfere with each other, such as having complimentary sequences to thereby be able to form an unwanted duplex structure when mixed together, S200 can be carried out in different manners.

According to some embodiments, the first RNA probe set and the second RNA probe set corresponding to a pair of RNA probe sets do not have substantially complimentary sequence (such as in one above-mentioned embodiment where the first RNA probe set and the second RNA probe set corresponding to a pair of RNA probe sets target two different portions of the duplex segment of the target nucleic acid sequence), or are conjugated to a solid support that do not readily interfere with each other (such as in one above-mentioned embodiment where the first RNA probe set and the second RNA probe set corresponding to a pair of RNA probe sets are conjugated to different regions of a microfluidic channel on a microfluidic chip), step S200 can comprise:

S200a: Contacting both the first RNA probe set and the second RNA probe set with the at least one target nucleic acid sequence in a single hybridization reaction.

According to some other embodiments, the first RNA probe set and the second RNA probe set in each pair have substantially complimentary sequences and thus can interfere with each other in a single hybridization reaction with the target nucleic acid sequences, as such, different embodiments of the method can be employed.

According to some embodiments, the first RNA probe set and the second RNA probe set in each pair can be configured to hybridize with the at least one target nucleic acid sequence in a sequential manner. Herein the “sequential manner” is referred to as a manner where the first RNA probe set and the second RNA probe set in each pair are allowed to contact with, and thereby to hybridize with, the at least one target nucleic acid sequence in the biological sample one after another. There is no limitation to the specific order: for example, the first RNA probe set is added to the hybridization reaction first, followed by the second RNA probe set, or alternatively, the second RNA probe set is added to the hybridization reaction first, followed by the first RNA probe set.

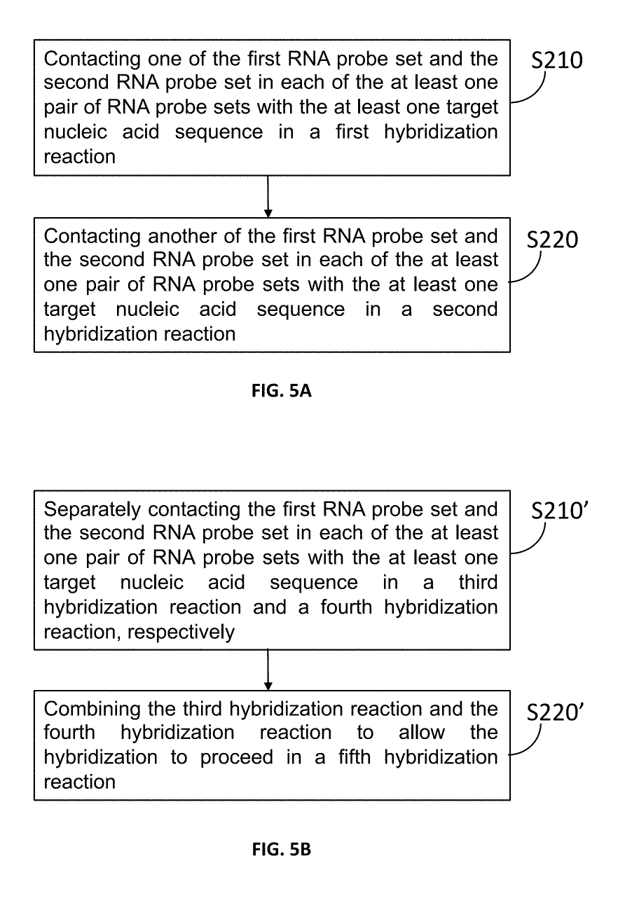

Specifically, according to some embodiments where the first RNA probe set and the second RNA probe set in each pair of RNA probe sets is labelled onto magnetic beads, the step S200 includes the following sub-steps, as illustrated in FIG. 5A:

S210: Contacting one of the first RNA probe set and the second RNA probe set in each of the at least one pair of RNA probe sets with the at least one target nucleic acid sequence in a first hybridization reaction; and

S220: Contacting another of the first RNA probe set and the second RNA probe set in each of the at least one pair of RNA probe sets with the at least one target nucleic acid sequence in a second hybridization reaction.

Alternatively, the first RNA probe set and the second RNA probe set in each pair can be configured to hybridize with the at least one target nucleic acid sequence in two separate hybridization reactions, which are then combined to react in another hybridization reaction. This is substantially a special situation for the sequential approach, and specifically, as shown in FIG. 5B, step S200 includes the following sub-steps:

S210′: Separately contacting the first RNA probe set and the second RNA probe set in each of the at least one pair of RNA probe sets with the at least one target nucleic acid sequence in a third hybridization reaction and a fourth hybridization reaction, respectively; and

S220′: Combining the third hybridization reaction and the fourth hybridization reaction to allow the hybridization to proceed in a fifth hybridization reaction.

Herein it is noted that “first”, “second”, “third”, “fourth”, and “fifth” are intended to refer to a different hybridization reaction and indicates or suggests no actual order in the reactions. It is further noted that any of the aforementioned hybridization reactions (i.e. the first, second, third, fourth, or fifth hybridization reaction) can occur at a temperature that substantially allows each RNA probe to hybridize efficiently with a corresponding strand of each target nucleic sequence.

For example, in some embodiments, each RNA probe has a length of ˜100-˜150 nt, and the temperature a hybridization reaction can be at a range of ˜40-90° C., preferably at ˜62-70° C., and more preferably at ˜67.5° C. Such a hybridzation temperature allows a high efficiency and a balanced specificity for the capture and enrichment of target nucleic acid sequences (see below). However, it is noted that these embodiments serve as illustrating examples only, and do not limit the scope of the disclosure. Other hybridization conditions, for example, RNA probes of a different length, at different abundances, a hybridization temperature, etc., can also be applied depending on specific needs.

Furthermore, the incubation time for each hybridization reaction can vary, depending on different configurations. According to some embodiments where the hybridization reaction occurs between RNA probes carrying the biotin moiety and the target nucleic acid sequences in the sample, the incubation time can be 6-24 hours, and preferably 12 hours, to ensure an efficient hybridization of each RNA probe to its corresponding strand of the target nucleic acid targets. According to some other embodiments where the hybridization reaction occurs between RNA probes conjugated onto an inside surface microfluidic channel and the target nucleic acid sequences in the sample, the incubation time can be several seconds to several hours, depending on the temperature and the pressure for the reaction. There are no limitations herein.

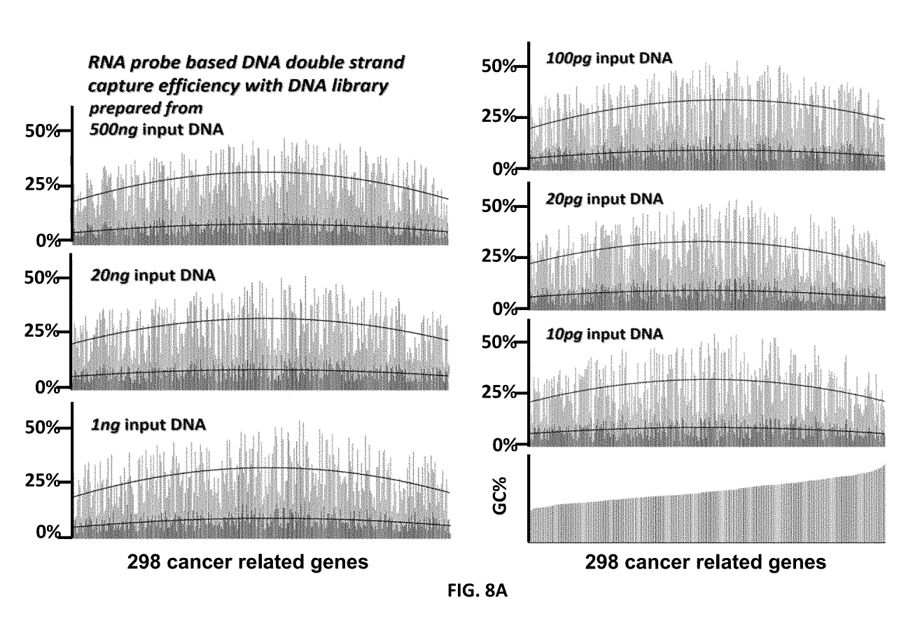

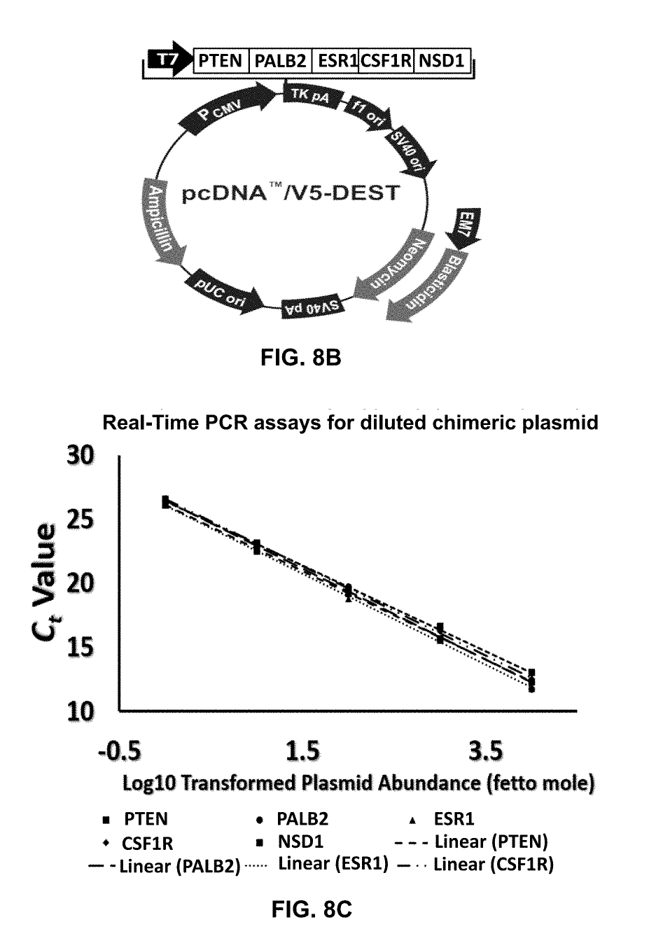

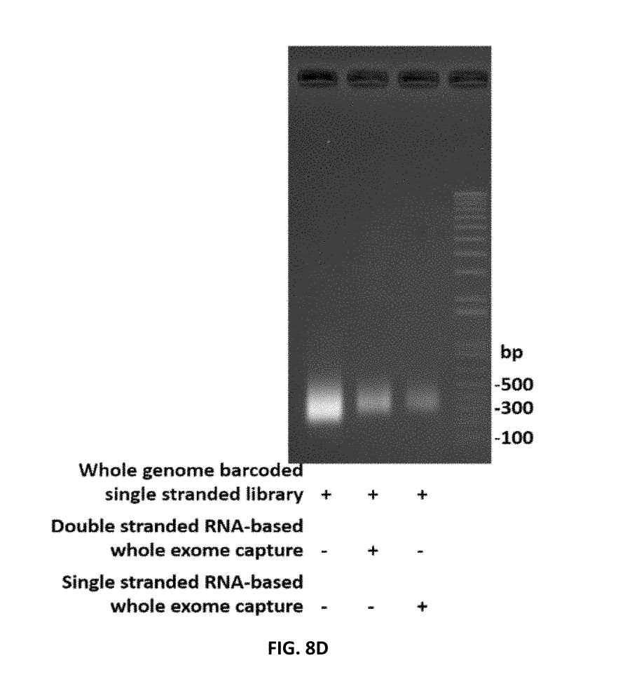

By sequentially adding the two RNA probe sets (i.e. the first RNA probe set and the second RNA probe set) in each pair in the hybridization reaction as described in the above embodiments, the unwanted formation of probe-probe hybridizations between themselves if simultaneously added can be effectively reduced. In addition, the sequential approach as described above has been proved to be more efficient in capturing target nucleic acid sequences. As has been demonstrated in an experiment that is described below, the capturing of a target DNA sequence utilizing a pair of complementary RNA probes that target both strands of the target DNA sequence can achieve an over 3-fold increase in the capture efficiency compared to the approach utilizing only one RNA probe that targets one single strand of the same target DNA sequence, as illustrated in FIG. 8A which will be described in detail below.

It is noted that in the above mentioned two approaches as illustrated in FIGS. 5A and 5B, the first RNA probe set and the second RNA probe set in each pair, once allowed to contact with the biological sample containing the at least one target nucleic acid sequence, are not removed from the reaction. Other embodiments are also possible.

For example, after each of the first RNA probe set and the second RNA probe set in each pair has contacted with the biological sample containing the at least one target nucleic acid sequence, each of the first RNA probe set and the second RNA probe set can be separated from the biological sample, allowing a capture of a portion of the at least one target nucleic acid sequence by the RNA probes in each of the first RNA probe set and the second RNA probe set, and the biological sample can be allowed to contact with each of the first RNA probe set and the second RNA probe set in each pair again.

It is noted that these embodiments actually allow the biological sample containing the at least one target nucleic acid sequence to repeatedly contact with the first RNA probe set and the second RNA probe set in each pair of RNA probe sets corresponding thereto. As such, the capture of the at least one target nucleic acid sequence from the biological sample can have a relatively higher efficiency after multiple rounds of sequential contact.

As such, the step S200 can include:

S211: Sequentially contacting the at least one target nucleic acid sequence with one and another of the first RNA probe set and the second RNA probe set in each of the at least one pair of RNA probe sets; and

optionally S212: Repeating S211 for at least one more time.

In one specific embodiment, the first RNA probe set and the second RNA probe set in each corresponding pair are respectively labelled onto magnetic beads, and as such, step S200 specifically comprises:

S211a: Contacting one of the first RNA probe set and the second RNA probe set with the biological sample containing the at least one target nucleic acid sequence;

S211b: Removing the one of the first RNA probe set and the second RNA probe set from the biological sample;

S211c: Contacting another of the first RNA probe set and the second RNA probe set with the biological sample; and

S211d: Removing the another of the first RNA probe set and the second RNA probe set from the biological sample.

These sub-steps S211a-S211d can be performed for only one time, or optionally can be repeated for at least one more time (i.e. S212).

In another specific embodiment, the first RNA probe set and the second RNA probe set in each corresponding pair are respectively conjugated onto the solid support (e.g. a matrix or a surface), which are respectively arranged at two different regions of an apparatus, such as a column or a microfluidic channel of a microfluidic chip. In one example as illustrated in FIG. 1D, a matrix conjugated with the first RNA probe set and a matrix conjugated with the second RNA probe set can be arranged at two different layers (10A and 10B) of a column. In another example as illustrated in FIG. 1E or FIG. 1F, the first RNA probe set and the second RNA probe set in each corresponding pair can be respectively immobilized onto two different regions of a microfluidic channel or chip along the direction of flow.

The apparatus (i.e. the column or the microfluidic channel) can be further configured to allow the biological sample to flow sequentially through the two different regions of the apparatus to thereby allow the biological sample containing the at least one target nucleic acid sequence to sequentially contact with the first RNA probe set and the second RNA probe set in each pair, which further supports repeated contact/separation to thereby increase the capture efficiency.

As such, step S200 can comprise:

S211a′: Allowing the biological sample containing the at least one target nucleic acid sequence to sequentially flow through the two different regions of the apparatus to thereby allow a sequential contact thereof with, one and another of the first RNA probe set and the second RNA probe set; and

Optionally S212′: Repeating S211a′ for at least one more time.

It is noted that in the embodiment having S211a′ and S212′ where a column or a microfluidic channel is employed, it is possible that the repeating step (i.e. S212′) is realized by arranging more than one pair of the first RNA probe set and the second RNA probe set in series on the column or the microfluidic channel along a direction of flow (illustrated in FIG. 1F), by arranging the sample to recirculate (i.e. by arranging the sample to flow into the column and the microfluidic channel again after flow out for more than one rounds, as illustrated in FIG. 1G), or by a combination of these two above approaches as well.

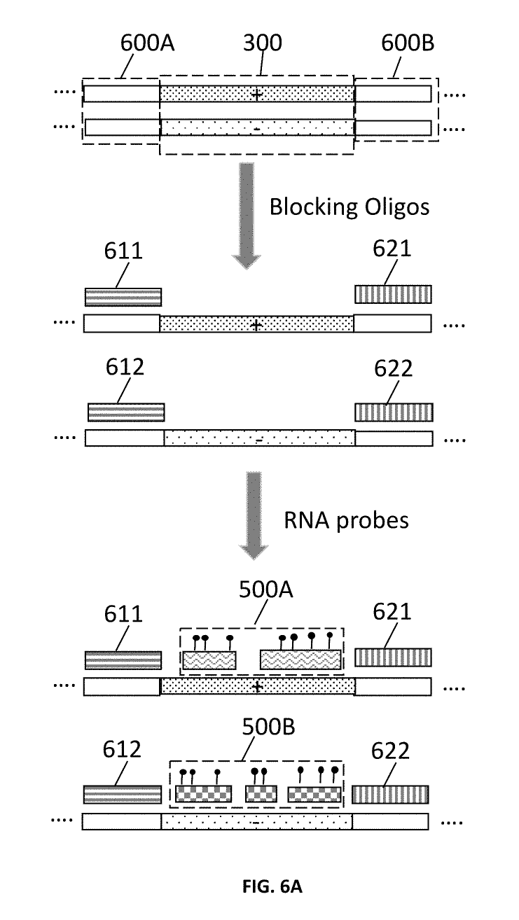

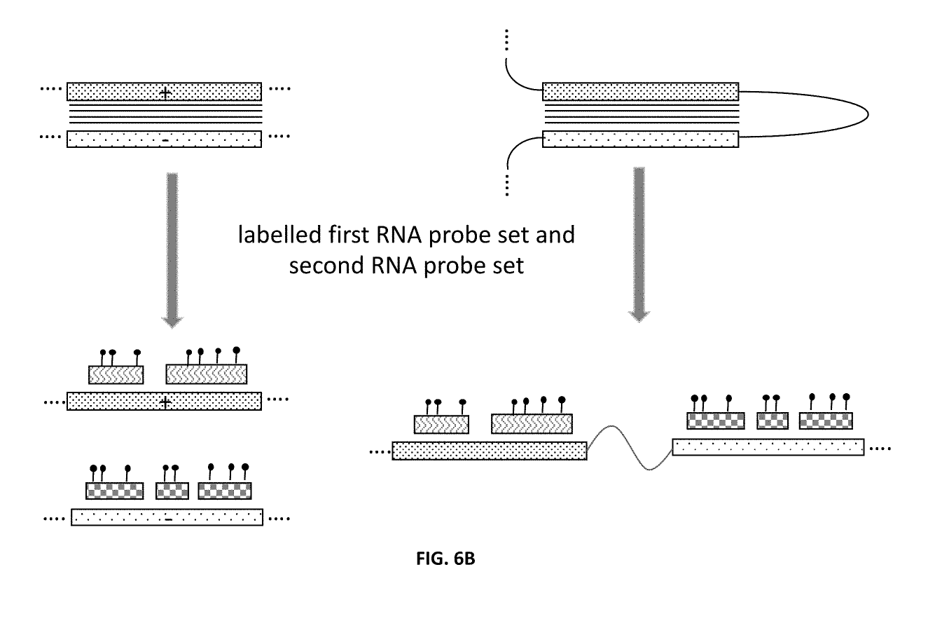

According to some embodiments of the disclosure, one or more target nucleic acids sequence in the biological sample may each be present in a polynucleotide which also contains at least one not-desired-to-be-captured sequences (termed “un-targeted sequence” hereafter). For example, in one specific embodiment as illustrated in FIG. 6A, the at least one target nucleic acid sequence is in a DNA library, and each target nucleic acid sequence is flanked by a pair of adaptor sequences, such as PE sequencing adapters having a length of ˜70 bp, which are substantially the un-targeted sequences. The presence of the at least one un-targeted sequence (e.g. one or two adaptor sequences) may potentially interfere with the enrichment or capture of the at least one target nucleic acid sequence by the RNA probes.

As such, in order to effectively eliminate the interference of the at least one un-targeted sequence, according to some embodiments of the disclosure, prior to step S200, the method further comprises:

S199: contacting at least one blocking oligo with the at least one target nucleic acid sequence, such that the at least one blocking oligo respectively hybridizes with, and thereby blocks, at least one strand of each of the at least one un-targeted sequence.

In one example, each target nucleic acid sequence is a double-stranded DNA sequence flanked by a pair of adaptor sequences (i.e. a first adaptor sequence 600A and a second adaptor sequence 600B as illustrated in FIG. 6A). As such, a set of blocking oligos comprising a first blocking oligo specifically targeting one strand of the first adaptor sequence 600A and a second blocking oligo specifically targeting one strand of the second adaptor sequence 600B, can be utilized in sub-step S201 to facilitate the hybridization of corresponding RNA probes with the each target nucleic acid sequence without the interference from the un-targeted sequences (i.e. the flanking adaptor sequences).

Herein, it is noted that there can be multiple arrangements for the set of blocking oligos. For example, the set of blocking oligos can consist of two blocking oligos, and can have a blocking oligo pair of (611 and 621), (611 and 622), (612 and 621), or (612 and 622), as long as the two blocking oligos target the two adaptor sequences 600A and 600B respectively, as shown in FIG. 6A. Alternatively, the set of blocking oligos can consist of three blocking oligos, having a combination of (611, 612 and 621), (611, 612 and 622), (611, 621 and 621), or (612, 621 and 622). Alternatively, the set of blocking oligos can consist of four blocking oligos (611, 612, 621 and 622), which substantially form two pairs of blocking oligos, 611/612 and 621/622, each pair corresponding to two strands of the first adaptor sequence 600A and two strands of the second adaptor sequence 600B, respectively, as illustrated in FIG. 6A.

It is noted that in order to avoid the unwanted annealing, hybridization, or binding, between two blocking oligos that target the two complimentary strands in any of the two adaptor sequences (600A or 600B), the two oligos can be added for hybridization and blocking in a sequential manner, or in a separate-and-combining manner, just as the addition of the first RNA probe set and the second RNA probe set in each of the at least one pair of RNA probe sets as illustrated in FIG. 5A or FIG. 5B.

Multiple embodiments for the at least one blocking olio are possible. For example, each of the at least one blocking oligo employed in S199 can be a single-stranded DNA oligo, or a single-stranded RNA oligo, which has a length of at least 2 nt and can be obtained based on a conventional technology known by people of ordinary skills in the field.

According to some embodiments, the at least one blocking oligo can comprise one or more blocking oligo sets, configured such that each blocking oligo set comprises one or more oligos which specifically target one of the two antiparallel strands of each of the at least one un-targeted sequence in a target nucleic acid sequence.

In one illustrating example of the at least one blocking oligo, it can be configured such that two blocking oligos (or two blocking oligo sets) respectively target two different portions of a duplex segment of each of the one or two adaptor sequences in a target nucleic acid sequence (i.e. a first portion in a first strand and a second portion in a second strand, where the first strand and the second strand are the two antiparallel strands in a duplex segment of each of the one or two adaptor sequences in the target nucleic acid sequence, and the first portion and the second portion are two different portions of the duplex segment of the target nucleic acid sequence.) This arrangement allows the simultaneous addition of the at least one blocking oligo in a blocking reaction without temporal separation, and brings about convenience.

In another example, two blocking oligos (or two blocking oligo sets) respectively target a same portion of a duplex segment of each of the one or two adaptor sequences in a target nucleic acid sequence, and as such, the two blocking oligos (or oligo sets) need to be allowed to contact the at least one target sequence in the biological sample in a sequential manner, or in a separate and then combined manner.

FIG. 6A shows one specific embodiment as an illustrating example.

In order for the set of blocking oligos to hybridize with, and thereby to block, the corresponding strand in each of the pair of two adaptor sequences respectively, the blocking hybridization reaction can occur at a substantially same temperature as the aforementioned hybridization reactions respectively required for hybridizing the first RNA probe set 500A and the second RNA probe set 500B to each corresponding strand of each target nucleic acid sequence. As such, according to some embodiments, the blocking hybridization reaction can be at a range of ˜40-90° C., preferably at ˜62-70° C., and more preferably at ˜67.5° C.

As such, by applying a corresponding set of blocking oligos 611, 612, 621, and/or 622 to block each of the two strands of the first adaptor sequence 600A and each of the two strands of the second adaptor sequence 600B that flank each of the at least one target nucleic acid sequence in the DNA library in the sample (as illustrated in FIG. 6A), the potential interference by each strand of the first adaptor sequence 600A and the second adaptor sequence 600B can be minimized.

According to some other embodiments, only one adaptor sequence is next to each double-stranded target nucleic acid sequence, and as such, one blocking oligo (which targets one of the two strands of the adaptor sequence) or a set of two blocking oligos (which respectively target two strands of the adaptor sequence) can be used.

According to yet some other embodiments, each target nucleic acid sequence is single-stranded in the biological sample and is flanked by two adaptor sequences, and as such, a set of two blocking oligos respectively targeting the two adaptor sequences can be used. There are other possibilities as well, and the specific design is dependent on actual situation. There are no limitations herein.

It is noted that one or more adaptor sequences as described above serve only as illustrating examples for the at least one un-targeted sequence in a polynucleotide sequence containing a target nucleic acid sequence, and do not limit the scope of the disclosure.

S300: immobilizing the at least one target nucleic acid sequence on a solid support;

Herein step S300 can be performed by means of the immobilization portion in the at least one functional portion that has been labelled onto each of the at least one RNA probe, as mentioned above.

Specifically, S300 can be carried out by means of a stable binding (i.e. non-covalent binding) between the immobilization portion in each RNA probe and a coupling partner immobilized on a surface of a solid support. In some specific embodiments where the immobilization portion is a biotin moiety, the coupling partner can be a streptavidin, avidin, or an anti-biotin antibody, attached onto, or conjugated with a solid support such as magnetic beads. Other examples of the immobilization portion-coupling partner pair can include, but is not limited to, a carbohydrate-lectin pair, an antigen-antibody pair and a negative charged group-positive charged group static interacting pair.

Alternatively, S300 can be carried out by means of a cross-link (i.e. covalent connection) between the immobilization portion and a coupling partner attached onto a solid support. In some specific embodiments, the immobilization portion can be a first coupling partner, which can form a cross-link with a second coupling partner, allowing for the further immobilization of the captured sequences. As such, the first coupling partner and the second coupling partner are respectively one and another of a cross-linking pair, selected from one of an NHS ester-primary amine pair, a sulfhydryl-reactive chemical group pair (e.g. cysteines, or other sulfhydryls such as maleimides, haloacetyls, and pyridyl disulfides), an oxidized sugar-hydrazide pair, photoactivatable nitrophenyl azide's UV triggered addition reaction with double bonds leading to insertion into C—H and N—H sites or subsequent ring expansion to react with a nucleophile (e.g., primary amines), or carbodiimide activated carboxyl groups to amino groups (primary amines), etc. There are no limitations herein.

By means of this step of the method, the target nucleic acid molecules are isolated, enriched, or captured from the biological sample via the labelled RNA probes. Solid supports that can be used may be any that are convenient for the particular purpose and situation.

S400: eluting out the at least one target nucleic acid sequence from the solid support.

After the immobilization of the at least one target nucleic acid sequence to the solid support through the at least one immobilization portion-labelled RNA probe in step S300, the plurality of immobilized target nucleic acid sequences are enriched or captured, which can then be eluted from the solid support in step S400 to facilitate the subsequent treatment and analysis, such as PCR amplification, a sequencing assay (such as next generation sequencing and other sequencing assays), PCR-based detection, microarray assays, construction of gene fragments into clones, transfection and transduction, and all other nucleic acid based applications. There are no limitations herein.

According to some embodiments of the method, the step S400 can include a washing process followed by an elution process. In the washing process, the hybridized molecules extracted by the solid supports can be washed to remove unspecific nucleic acid binders, including nucleic acids that are binding to probes or binding to solid supports or binding to any other moieties due to unspecific interactions. Specifically, the washing process can be carried out by saline-sodium citrate (SSC) buffer with 0.1% SDS.

In the elution process, nucleic acid sequences that are hybridized to the RNA probes can be eluted through heat-induced strand dissociation or through nucleic acid denaturing reagents, such as 0.1M sodium hydroxide. After separation of the probe from the target nucleic acid molecule through elution, a neutralizing buffer, such as Tris-HCl pH7.5, can be used to treat the target nucleic acid molecule portion of the elution reaction to further neutralize the effects initiated by the denaturing buffer if initially utilized.

According to some embodiments of the method, rather than immobilizing the at least one target nucleic acid sequence on the solid support (i.e. step S300) after hybridizing the RNA probes generated by step S100 with the at least one target nucleic acid sequences (i.e. step S200), the RNA probes generated by step S100 can be first immobilized on the solid support (and thus become solid support-immobilized RNA probes or solid support-conjugated RNA probes), and then can be allowed to contact with the target nucleic acid sequences for capturing. As such, the method can comprise the following steps, as illustrated in FIG. 1B:

S100′: Preparing at least one pair of RNA probe sets, each pair comprising a first RNA probe set and a second RNA probe set configured to respectively target two antiparallel strands of a duplex segment in each of at least one target nucleic acid sequence, wherein each RNA probe in any of the first RNA probe set and the second RNA probe set is labelled with an immobilization portion;

S200′: Conjugating each of the at least one pair of RNA probe sets on a solid support;

S300′: Contacting one and another of the solid support-conjugated first RNA probe set and the solid support-conjugated second RNA probe set with the at least one target nucleic acid sequence to allow hybridization of each RNA probe in the at least one pair of RNA probe sets to a corresponding strand of each target nucleic acid sequence; and

S400′: Eluting out the at least one target nucleic acid sequence from the solid support.

Herein, steps S100′ and S400′ in the embodiments of the method as described above are substantially same as steps S100 and S400 in the aforementioned embodiments of the method as illustrated in FIG. 1A. The solid support can be magnetic beads, non-magnetic beads, resin matrix, filter, membrane, or a different type that has been mentioned above.

Specifically, in a manner similar to the above embodiments where the RNA probes are immobilized to the solid support only after their hybridizations with target nucleic acid sequences, a pair of solid support-immobilized RNA probe sets (i.e. the solid support-conjugated first RNA probe set and the solid support-conjugated second RNA probe set in the pair) can be allowed to sequentially contact with the at least one target nucleic acid (i.e. one after another), or to separately contact with the at least one target nucleic acid in two hybridization reactions and then to combine the two hybridization reactions.

As such, according to a first embodiment which is substantially a sequential manner, S300′ includes:

S310: Contacting one of the solid support-conjugated first RNA probe set and the solid support-conjugated second RNA probe set with the at least one target nucleic acid sequence in a sixth hybridization reaction; and

S320: Contacting another of the solid support-conjugated first RNA probe set and the solid support-conjugated second RNA probe set with the at least one target nucleic acid sequence in a seventh hybridization reaction.

According to a second embodiment, S300′ includes:

S310′: Separately contacting the solid support-conjugated first RNA probe set and the solid support-conjugated second RNA probe set in each pair of solid support-conjugated RNA probe sets with the at least one target nucleic acid sequence in an eighth hybridization reaction and a ninth hybridization reaction, respectively; and

S320′: Combining the eighth hybridization reaction and the ninth hybridization reaction to thereby allow a tenth hybridization reaction to proceed.

It is noted that depending on the specific properties of the solid support, it is possible that a pair of RNA probe sets conjugated on the solid support have little or acceptable level of interactions among RNA probes having complimentary sequences. As such, according to a third embodiment, a solid support-conjugated first RNA probe set and a solid support-conjugated second RNA probe set in each corresponding pair can be allowed to contact with the at least one target nucleic acid in a single hybridization reaction without being separated temporally. For example, if beads are used, the beads-conjugated first and second RNA probe set in each corresponding pair can be combined in a single reaction for the simultaneous capture of different strands of a duplex segment of a target nucleic acid sequence. Alternatively, a glass surface can be used for conjugation of both the first and the second RNA probe set in each corresponding pair thereon and can allow a simultaneous capture of different strands of a duplex segment of a target nucleic acid sequence in a single reaction.

It is further noted that according to a fourth embodiment, a first RNA probe set and a second RNA probe set in each corresponding pair can be conjugated at a different region of the solid support, such as at a different segment of a microfluidic channel on a microfluidic chip, which allows a sample containing the at least one target nucleic acid sequence to sequentially flow through the different segments of the microfluidic channel corresponding to the pair of solid support-conjugated RNA probe sets to thereby allow a sequential capture of the two different strands of a duplex segment of a target nucleic acid sequence. Such a configuration allows a repeated use of RNA probes conjugated on the solid support.

According to some embodiments of the method, rather than conjugating each of the at least one pair of RNA probe sets on a solid support after preparing the at least one pair of RNA probe sets (i.e. steps S100′ and S200′ in the above mentioned method shown in FIG. 1B), the RNA probes in each of the at least one pair of RNA probe sets can be directly prepared on the solid support. As such, the method can comprise the following steps, as illustrated in FIG. 1C:

S100″: Preparing at least one pair of RNA probe sets directly on a solid support, each pair comprising a first RNA probe set and a second RNA probe set configured to respectively target two antiparallel strands of a duplex segment in each of at least one target nucleic acid sequence;

S200″: Contacting one and another of the solid support-conjugated first RNA probe set and the solid support-conjugated second RNA probe set with the at least one target nucleic acid sequence to allow hybridization of each RNA probe in the at least one pair of RNA probe sets to a corresponding strand of each target nucleic acid sequence; and

S300″: Eluting out the at least one target nucleic acid sequence from the solid support.

Herein, steps S200″ and S300″ in the embodiments of the method as described above are substantially same as steps S300′ and S400′ in the aforementioned embodiments of the method as illustrated in FIG. 1B. The solid support can be magnetic beads, non-magnetic beads, resin matrix, filter, membrane, or a different type that has been mentioned above.

Herein, step S100″ can be realized by direct chemical synthesis or by direct transcription on the solid support. In cases where the direct transcription on the solid support, a RNA polymerase can be attached onto the solid support.

The method, as illustrated by the various embodiments as described above, has the following advantageous features:

First, a pair of RNA probes which respectively target two antiparallel strands (i.e. a plus strand and a minus strand) of a duplex segment of each target nucleic acid sequence are both employed for the enrichment or capture of both strands of each of the target nucleic acid sequences, resulting in a higher enrichment efficiency compared with a conventional method where there is only one set of RNA probes targeting only one of the two strands of each target nucleic acid sequence.

Second, the nucleic acid sequence capture approach as described above can substantially capture both of the two antiparallel strands that typically belong to a same molecule of each target nucleic acid sequence and have complementary sequences, and can thus bring additional advantages in certain applications having a high requirement for sequence accuracy, such as single-nucleotide variation (SNV) calling. In an exemplary application in detecting SNVs in a sample, only an SNV observed on both of the two complementary strands in a target DNA sequence that have been respectively captured by a corresponding pair of RNA probe sets respectively targeting both of the two complementary strands is a bona fide SNV, whereas an SNV observed only on one, but not on both, of the two complementary strands in a target DNA sequence is likely to represent a false positive signal introduced by PCR. As such, the nucleic acid sequence capture approach disclosed herein allows for an error-proof application.

Third, each RNA probe can be prepared by transcription, which allows a large amount of RNA probes to be cost-effectively and conveniently obtained. This allows the target nucleic acid sequences to be enriched/captured at a significantly higher efficiency due to the much higher probe/target ratio. Additionally, this further allows a quantification of each RNA probe, in turn causing each RNA probe to be conveniently tweaked to increase the efficiency of capturing some specific target nucleic acid sequences that are difficult to capture by, for example, using a 10-fold higher amount of RNA probes for capturing.

Fourth, the above advantageous features together can have an additional advantage such that the hybridization of a first RNA probe with its corresponding strand of each target nucleic acid sequence could help expose the other complementary strand of each target nucleic acid sequence, thereby could kinetically favor the subsequent hybridization of another RNA probe with the complimentary strand of the each target nucleic acid sequence, leading to a favorable capturing of each of the two strands of each target nucleic acid sequence in the biological sample.

Due to these above advantages, the method disclosed herein is expected to have a variety of applications, as follows:

First, it allows for the capture and analysis of sequence variants and/or copy number variants, including without limitation, a point mutation, a deletion, an amplification, a loss of heterozygosity, a rearrangement, and/or a duplication. These genetic variants may be associated with human diseases, specific phenotypes, etc. The analyses include sequencing, hybridization assay, ligation assay, etc. Due to its ultra-sensitivity, the method disclosed herein allows for the capture and analysis of rare variants, especially ultra-rare variants/mutations, and copy number variants, and can be employed in capture and enrichment of nucleic acids from mitochondria, chloroplast, plastid, bacterial or viral pathogens, environmental DNA (eDNA), etc., and can also be employed in capture and enrichment of nucleic acids for population genetics studies, SNP typing and deep phylogenies, RAD (Restriction-site-Associated DNA sequencing) or GBS (Genotyping By Sequencing) locus enrichment. There is no limitation herein.

Second, it allows for enrichment and molecular cloning of specific target nucleic acid sequences blended in a DNA sample, such as ancient DNA and nucleic acid materials from museum specimens, which typically have a poor DNA quality and have a typically serious contamination of other organisms (esp. microorganisms) over many years' storage in nature environment, or contamination of modern human DNA due to inappropriate sample handlings.

Third, the method can be applied broadly to capture a variety of nucleic acid sequences in a test sample, which can include a double-stranded nucleic acid sequence, which consists of a plus strand and a minus strand, as illustrated in FIG. 6B, left panel), and can also include a single-stranded nucleic acid sequence having a duplex segment, which substantially forms a loop or a hairpin (as illustrated in FIG. 6B, right panel), and the nucleic acid sequences can include DNAs, RNAs, or DNA-RNA hybrid molecules.

It is noted that even in situations where any of the target nucleic acid sequences does not form a duplex structure (i.e. the target nucleic acid sequence has only a single-stranded segment), such as a case where cDNA is synthesized via reverse transcription yet the mRNA template is degraded due to an RNase-induced digestion, the method as described above can still be applied to capture a target nucleic acid sequence as long as any of the first RNA probe set or the second RNA probe set can target the single-stranded segment of the target nucleic acid sequence.

Fourth, the method as disclosed herein, if combined with the use of a method and a kit for constructing a barcoded nucleic acid library as disclosed in the U.S. patent application Ser. No. 15/908,190 (i.e. if used to enrich and capture target nucleic acid sequences in the barcoded DNA library by the kit and method disclosed therein), can allow an ultra-sensitive error-proof assays for the detection and characterization of target nucleic acid sequences in a biological sample.

In the following, several illustrating examples are provided to illustrate the several applications of the method disclosed herein.

FIG. 7A and FIG. 7B show a diagram of the nucleic acid sequence enrichment method according to some embodiments of the present disclosure.

As illustrated in FIG. 7A and FIG. 7B, double-stranded DNA molecules in a NGS DNA library are dissociated and the shared adapter, index and universal primer sequences among all molecules were hybridized by blocking oligos, and the target DNA sequences are further captured by RNA probes that are complementary to the DNA molecules that are composed of antiparallel double strands. Both the + strand and the − strand from the same DNA molecule are captured respectively by at least one complementary RNA probe. After immobilization of the captured DNA sequences on the solid support (i.e. streptavidin magnetic beads), the target sequences are extracted from the original library and the target DNA molecules being captured can be eluted from the probe after a series of wash and elution steps. After amplification, the library is ready for direct NGS sequencing or other assays.

Any means of testing for a sequence variant or sequence copy number variant, including without limitation, a point mutation, a deletion, an amplification, a loss of heterozygosity, a rearrangement, a duplication, may be used. Sequence variants may be detected by sequencing, by hybridization assay, by ligation assay, etc. The defined locations of some mutations permit focused assays limited to an exon, domain, or codon. But un-targeted assays may also be used, where the location of a mutation is unknown. If locations of the relevant sequence variants are defined, specific assays which focus on the identified locations may be used. Any assay that is performed on a test sample involves a transformation, for example, a chemical or physical change or act. Assays and determinations are not performed merely by a perceptual or cognitive process in the body of a person.

Probes and/or primers and/or template for RNA probe synthesis may contain the wild-type or a sequence variant, including without limitation, a point mutation, a deletion flanking sequence, a rearrangement location, may be used. These can be used in a variety of different assays, as will be convenient for the particular situation. Selection of assays may be based on cost, facilities, equipment, electricity availability, speed, reproducibility, compatibility with other assays, invasiveness of sample collection, sample preparation, etc.

Any of the assay results may be recorded or communicated, as a positive act or step. Communication of an assay result, diagnosis, identification, or prognosis, may be, for example, orally between two people, in writing, whether on paper or digital media, by audio recording, into a medical chart or record, to a second health professional, or to a patient. The results and/or conclusions and/or recommendations based on the results may be in a natural language or in a machine or other code. Typically, such records are kept in a confidential manner to protect the private information of the patient or the project.

Collections of RNA probes, primers, control samples, and reagents can be assembled into a kit for use in the methods. The reagents can be packaged with instructions, or directions to an address or phone number from which to obtain instructions. An electronic storage medium may be included in the kit, whether for instructional purposes or for recordation of results, or as means for controlling assays and data collection.

Control samples can be obtained from the same patient from a tissue that is not apparently diseased. Alternatively, control samples can be obtained from a healthy individual or a population of apparently healthy individuals. Control samples may be from the same type of tissue or a different type of tissue than the test sample. Control samples may be provided together with the RNA probes, primers, and reagents in a kit for use in the method, where the control samples may be a standard reference sample for the purpose of validating the performance of the kit and the operation performed by the user.

The data described below document the results for the identification of ultra-rare mutations from a whole exome sequencing study using RNA probes targeting both strands of the target DNA molecules. There is no doubt that SNVs can be detected with confidence only when the sequencing system's error rate is significantly lower than the frequency of identified SNVs. Therefore, baseline error rate of an NGS pipeline is critical for its performance of detecting ultra-rare SNVs. Combing the RNA probe-based capture of both DNA strands with the single-stranded library construction, an improved NGS methods with the base line error rate as 2.25×10⁻¹⁰ was created. Such high-accuracy pipeline is dependent on the massive amount of DNA library target molecules captured by the RNA probes targeting both DNA strands.

FIGS. 8A-8D show that reduced amounts of variants were re-detected from sequentially diluted samples. No variant was re-detected from 1:10,000 diluted group. Coverage of re-sequencing is ˜5,000×. The efficiency of capture through a paired sets of RNA probes is significantly stronger than the efficiency of capture through a single set of RNA probe for any target sequence.

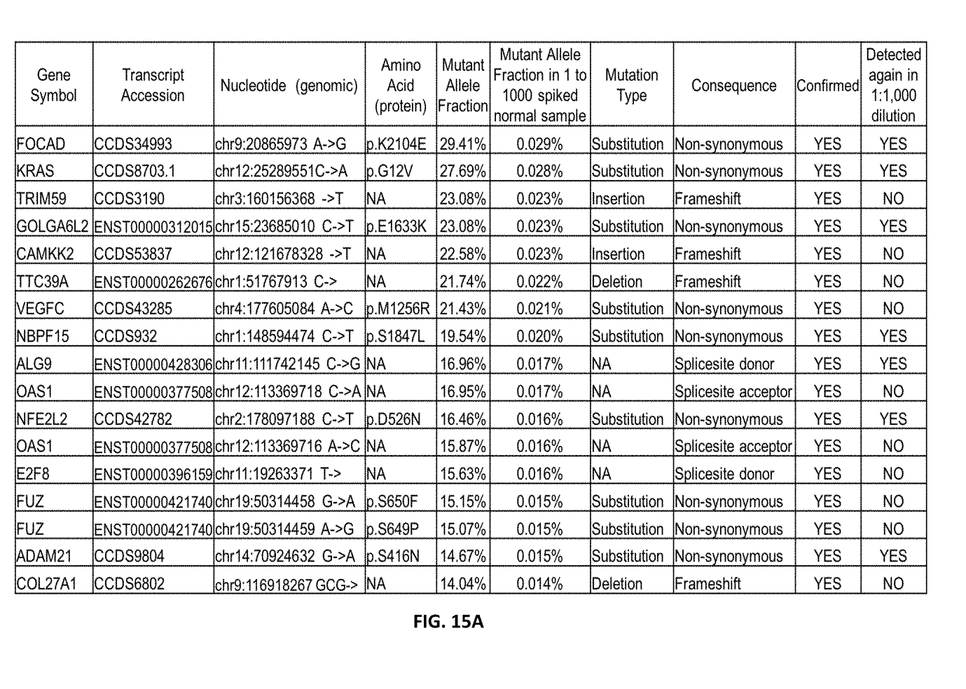

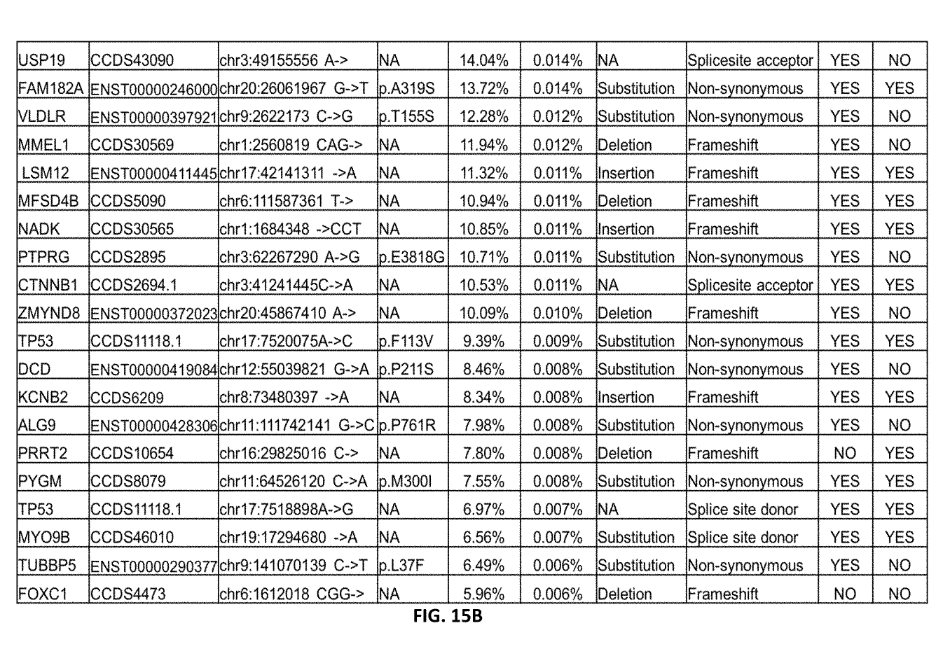

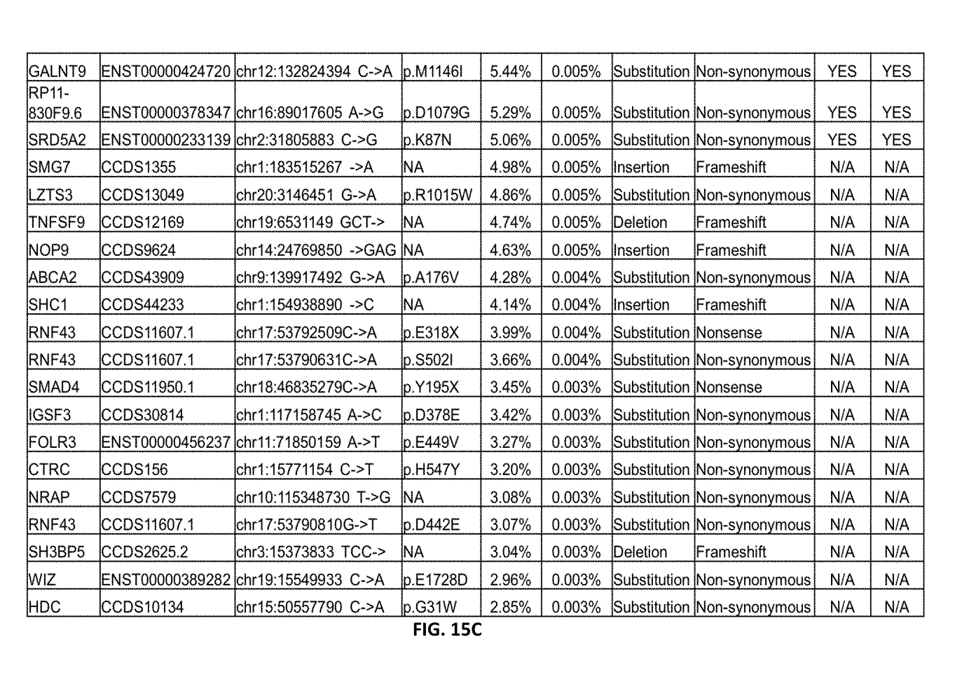

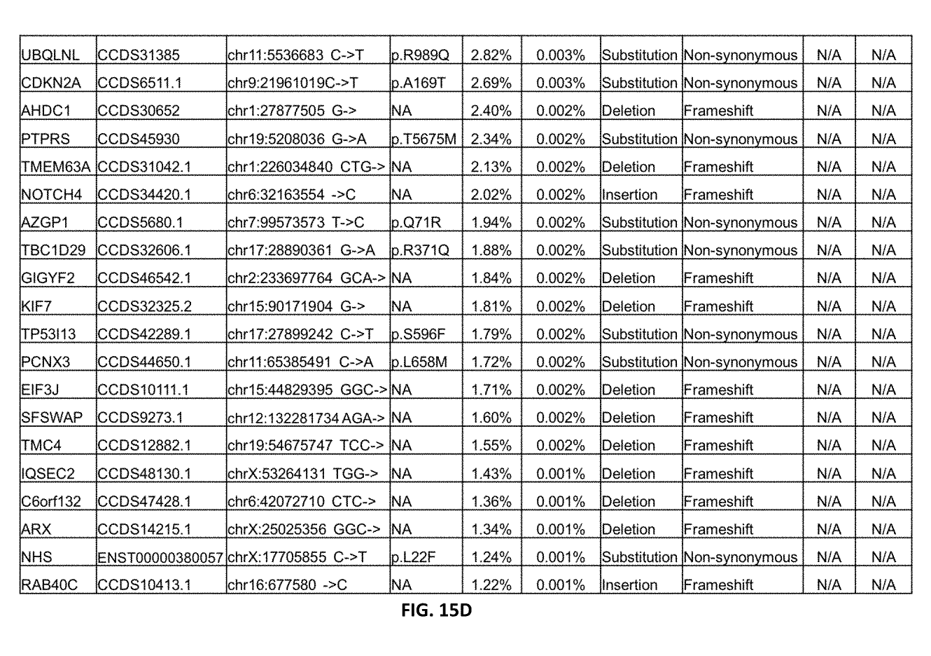

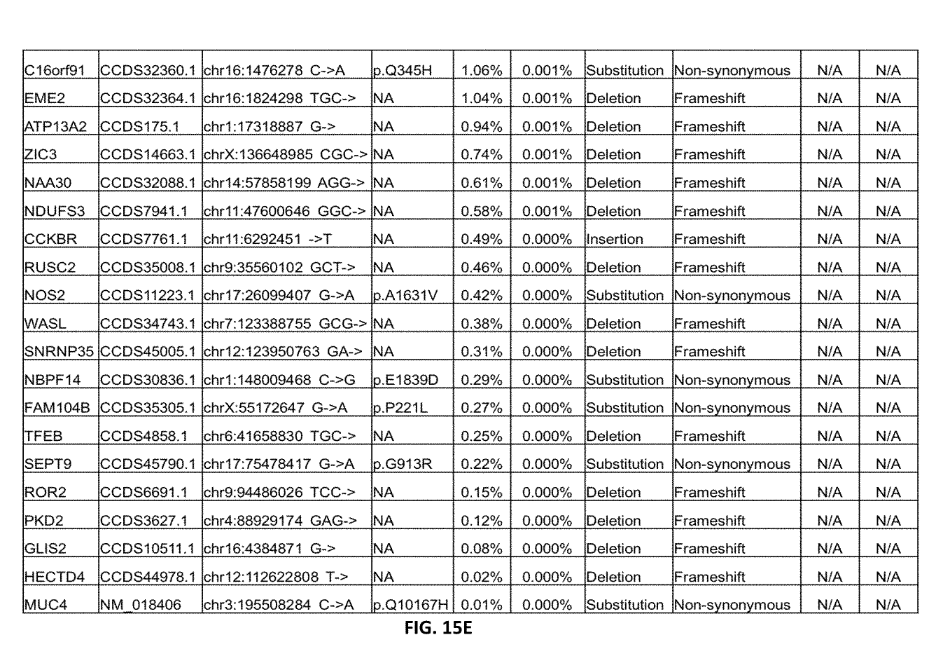

FIGS. 15A, 15B, 15C, 15D and 15E show sequence variants detected by RNA probe-based DNA double strand capture NGS, validation results by Sanger sequencing and ultra-rare mutation redetection results are shown and ranked by Mutant Allele Fraction.

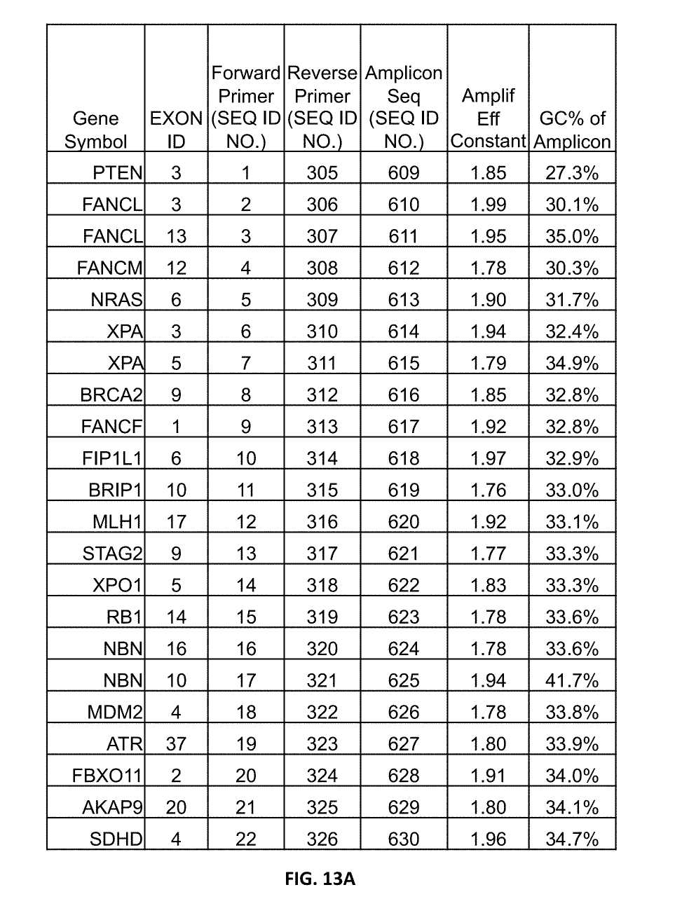

The ultra-rare mutation detection performance of this method with the target molecules captured by RNA probes targeting both strands of the DNA molecules was then evaluated by the success rate of re-detecting the 38 Sanger sequencing validated sequence variants in the libraries created from normal DNA samples which were spiked with sequential dilutions of tumor DNA. The library is constructed by a barcoded single-strand molecule-based approach and the target enrichment of the whole exome region of the human genome is performed by RNA probe-based DNA double strand capture. As the dilution folds increased, as expected, fewer and fewer variants were detected (FIG. 12), and when the tumor DNA sample was diluted 1,000 folds (the diluted sample containing 0.1 ng tumor DNA and 100 ng normal DNA), only 21 out of the 38 validated variants can be detected (FIGS. 15 A-E). The allelic fractions of these 21 SNVs in the 1:1000 diluted sample range from 0.03% to 0.005% with an average of 0.013% (FIGS. 15 A-E). No sequence variant was detected in 1:10,000 diluted sample which may presumably be due to the limitation of sequencing depth that has been achieved. For each sample, the targeted sequencing was performed with an average depth of 5,000×, which theoretically only allows us to see SNVs down to the frequency of 1/5000 (0.02%). To observe ultra-rare SNVs with even lower frequency, a greater than 5000× coverage is needed. It is also helpful to design capturing probes targeting only a small number of genes. With a smaller number of different nucleic acid sequences captured by the RNA probes targeting both strands of a smaller cohort of target genes, instead of the entire human genome exome, more copies of the target nucleic acids can be enriched, and this method can achieve a much greater sequencing depth with a significantly improved accuracy of ultra-rare SNV calling. The extremely low baseline error rate of this method allows ultra-rare SNV calling at the whole exome level with high accuracy, and the depth of NGS sequencing becomes the only limiting factor for such applications.

An RNA probe-based DNA double strand capture approach was reported as an improved method to enrich DNA molecules for NGS purpose, particularly targeted NGS. Such improved performance has been demonstrated in a human genome WES study. Aside from WES, another very important application of RNA probe-based DNA double strand capturing would be the targeted resequencing of a gene panel. Targeted re-sequencing is one of the most popular NGS applications, and it allows people to sequence a small cohort of gene targets to extreme depths, usually thousands of folds of coverage. And such sequencing depth can facilitate the detection of ultra-rare mutations with great sensitivity. In an RNA probe-based DNA double strand capture pipeline, attempts were made to capture the entire exome of all human genes, where an over 98% coverage with the depth of over 200× was achieved on a standard NGS platform. More importantly, the detection limit of this method for rare-mutation detection on whole exome scale is as low as 0.03%, which is made possible by the massively improved capture efficiency of the target molecules by the RNA probes targeting both DNA strands. For an even smaller cohort of target genes, the depth and coverage of RNA probe-based DNA double strand capturing NGS can be further increased, and the performance of ultra-rare mutation detection can be subsequently improved over several additional orders of magnitude.

Other than identifying ultra-rare SNVs with high sensitivity and accuracy, RNA based double strand capture method can also be adopted for gene copy number variant (CNV) assays. Barcoded single-stranded library construction links a unique barcode to every single-stranded DNA molecules. Such barcode information can not only be used to label the molecules and create super reads to reduce PCR errors, but also be used as a location marker for DNA fragments. After RNA probe-based DNA double strand capturing, NGS sequencing, and mapping the super reads back to the human genome, the barcode on each super read can be assigned to the position where the super read sequence is mapped. Therefore, a human genome can be reconstructed by unique barcodes. Copy number information can be represented by the diversity of barcodes at subgenomic loci. A highly efficient capture reaction with equal efficiency for all genomic regions offered by RNA probe-based DNA double strand capture is the key to a successful CNV calling.

Aside from CNV analysis, large structural variants frequently observed in cancer genomes can also be analyzed through RNA probe-based DNA double strand enrichment. RNA probes can be designed to enrich subgenomic regions flanking popular genome breakpoints specifically. A highly sensitive pipeline for translocation and large indel identification could be built based on the high efficiency of RNA probe-based DNA double strand capture.

In addition to applications in basic research, RNA probe-based DNA double strand capturing has a great potential in clinical NGS fields. It has been demonstrated that this method can highly efficiently construct NGS DNA libraries with very low amount of DNA materials ( [symbol] 20 pg). Meanwhile it can detect ultra-rare mutations with high confidence. Such features are critical for NGS based clinical diagnostics where the samples are often limited and highly heterogeneous. A typical example would be the NGS sequencing of FFPE samples. FFPE has been a standard sample preparation method for many decades. Historically archived FFPE sample is a very valuable resource for retrospective studies in biomedical research. However, due to chemical modifications during specimen preparation and chronic damages to the tissue blocks or slides over long-term storage, it has been a challenging task to conduct NGS studies with FFPE samples. Poor DNA quality and artificial sequence changes are two major issues coming along with FFPE based NGS studies. RNA probe-based DNA double strand capturing is offering great benefit for FFPE based WES studies. WES data have been reported to be discordant between FFPE and fresh frozen samples at lower coverage levels (˜20×), however, this discrepancy can be reduced when higher coverages are achieved. And recently, Allen et al. reported a reciprocal overlap of 90% somatic mutations between FFPE and fresh frozen tissue samples for the positions with sufficient sequencing (Van Allen, Wagle et al. 2014). In Allen's study, an RNA probe-based DNA single strand capture approach was applied, where its capture efficiency is far lower than RNA probe-based DNA double strand capture approach as was shown in data. With the enhanced capture efficiency by this method, WES studies with FFPE samples will offer comparable data quality to WES studies with fresh frozen tissues.

This method has a great potential to discover novel low-frequency disease-causing variants in biomedical and clinical applications, and can identify more actionable therapeutic targets for patients. This method can fulfill an unprecedented level of personalized precision medicine by revealing the most complete patient genomic profile to date including high-frequency, low-frequency and particularly ultra-low-frequency mutations. This method can also be applied in other clinical applications, like circulating DNA sequencing from body fluid samples, where only limited amount of DNA materials is available. In clinical NGS applications, it is critical to highly efficiently capture target DNA molecules from NGS libraries constructed with very limited amount of highly heterogeneous samples thus being less- or non-invasive; to highly efficiently enrich target sequences thereby reaching a great sequencing depth with limited cost and improved diagnostic sensitivity; and to remove artificial sequencing errors as completely as possible for the best diagnostic specificity. This method, utilizing two sets of RNA probes to capture the two strands of DNA antiparallel molecules simultaneously, has been demonstrated to meet these needs with great potentials in numerous NGS applications.

### Example 1

Materials and Methods

Tumor and Normal Tissue Sample

The paired tumor and normal tissue samples from a pancreatic cancer patient of Asian race were obtained in accordance with guidelines and regulations from Tianjin Medical University Cancer Institute & Hospital, P.R. China after Institutional Review Board (IRB) approval at Tianjin Medical University, and under full compliance with HIPAA guidelines. An informed consent for conducting this study was obtained from the patient. The tumor tissue sample has an estimated neoplastic content of 43.4%.

Library Preparation

Genomic DNA from patient normal and tumor fresh frozen tissues was extracted using DNeasy Blood & Tissue Kit (Qiagen) and sheared into 150 bp fragments with Diagenode's Bioruptor at a program of 7 cycles of 30 seconds ON/90 seconds OFF using 0.65 ml Bioruptor® Microtubes. Barcoded single-stranded library preparation starts from a complete dissociation of DNA duplex to form single-stranded DNA and tagging the 3′ end of each DNA single strand individually with a unique digital barcode. Barcoded single-stranded adapters have been disclosed in U.S. patent application Ser. No. 15/908,190, the disclosure is incorporated herein in its entirety. Pre-dephosphorylated fragmented DNA samples were mixed with barcoded single-stranded adapter (final concentration 0.15 uM), 20% PEG-8000, 100 U CircLigase II, and incubated at 60° C. for 1 hour. After immobilizing the ligation product on Streptavidin-coupled Dynabeads (ThermoFisher Scientific), each barcoded single-stranded DNA molecule is subject to an individual single-cycled PCR reaction to form its complementary strand. A DNA primer complimentary to the single-stranded adapter was annealed and extended using Bst 3.0 polymerase at 50° C. for 30 minutes. Blunt-end repair using T4 DNA polymerase was performed at 25° C. for 15 minutes. A double-stranded adapter was then ligated to the 5′ end of the DNA duplex using T4 DNA ligase with an incubation at 16° C. for 1 hour. The library is eluted from the beads by an incubation at 95° C. for 1 minute. High fidelity PCR amplification is performed to amplify the DNA sequence as well as the unique barcode. Adapter sequences are designed to be compatible with Illumina sequencing platforms.

RNA Probe Synthesis

To obtain RNA probes complementary to both strands of target subgenomic regions, particularly the exome regions, the entire exome sequences for every human gene were cloned by sequence synthesis and molecular cloning based on Hg19 reference human genome sequences. In brief, exome sequences in 32,524 CCDS IDs containing −50 bp and +50 bp intronic sequences were cloned into pcDNA 6.2 vector. For extremely large human genes, e.g. DMD, PTPRD, CNTNAP2, etc., their related target sequences were separated and subcloned into multiple vectors. The total DNA sequences used to generate RNA probes cover a 72.6 Mb genome region, where all the exomes with their −50 bp and +50 bp flanking intronic sequences, as well as 5′ and 3′ UTRs for each gene were included. Two clones for each target sequence were constructed, where a T7 promoter was inserted at the 5′ end of the plus strand in the “+” clone and at the 5′ end of the minus strand in the “−” clone (FIG. 4B and FIGS. 7A and 7B). Two pools of clones were established for any give number of genes following the rule that the two clones for the same DNA sequence are separated into two systems, where one system (FIG. 4B and FIGS. 7A and 7B, left panel, “+” Clone) produced the RNA probes targeting the plus strand of the DNA target, and the other system (FIG. 4B and FIGS. 7A and 7B, right panel, “−” Clone) produced the RNA probes targeting the minus strand of the DNA target through in vitro transcription. ATP, CTP, GTP, UTP, and Biotin-16/11-UTP were added in each transcription system at the concentration of 1 mM, 1 mM, 1 mM, 0.7 mM and 0.3 mM. RNA products were further sheared into 100-150 nt fragments with a Covaris S220 focused-ultrasonicator (Covaris). The fragmented RNA probes are ready for RNA probe-based DNA double strand capture applications. The two RNA probe libraries for each target DNA sequence were created separately and were never mixed until the actual capture procedure was carried out. In this study, RNA probes targeting both plus and minus strands of the whole exome sequences were created (including −50 bp and +50 bp flanking intronic sequences and 5′/3′ UTRs) for all human genes and a cancer-related 298-gene panel.

RNA Probe-Based DNA Double Strand Capture

RNA probe-based DNA double strand capture was performed to capture the whole exome of human genome following a library construction or a standard NGS library construction. In RNA probe-based DNA double strand capture, both DNA strands of the target regions are captured by a pair of complementary RNA probes where each DNA strand is targeted by its complementary RNA probe, individually. A hybridization mixture was prepared containing 500 ng DNA library, 2 ug of RNA probes (1 ug from “+” clone transcripts and 1 ug from “−” clone transcripts, targeting Hg19 human exomes including −50 bp and +50 bp flanking intronic sequences, as well as 5′ and 3′ UTRs as described before), 7 ul Human Cot-1 DNA (ThermoFisher Scientific), 3 ul Herring Sperm DNA Solution (ThermoFisher Scientific), 10 μl blocking Oligos (1 nmol/ul each) with following sequences:

Blocking Oligo 1:
5′-AAGAGCGTCGTGTAGGGAAAGAGTGTAGATCTCGGTG
GTCGCCGTATCATT-3′
Inverted dT (SEQ ID NO. 913, where the last T
is labelled with an inverted dT)
Blocking Oligo 2:
5′-CCTCAGCAAGAGCACACGTCTGAACTCCAGTCAC-NN
NNNNN-ATCTCGTATGCCGTCTTCTGCTTG-3′
Inverted dT (SEQ ID NO. 914, where N can
be any of A, T, C, and G, and the last G is
labelled with an inverted dT)

The hybridization mixture is heated for 5 minutes at 95° C., then held at 67.5° C. 25 ul pre-warmed (67.5° C.) 2.8× hybridization buffer (14×SSPE, 14×Denhardt's, 14 mM EDTA, 0.28% SDS) was added. The mixture was slowly pipetted up and down 8 to 10 times. The hybridization mixture was incubated for 24 hours at 67.5° C. with a heated lid.

After hybridization, 50 μl Dynal MyOne Streptavidin C1 magnetic beads (ThermoFisher Scientific) were washed three times by adding 200 μl of binding buffer (1M NaCl, 10 mM Tris-HCl, pH 7.5, and 1 mM EDTA), and re-suspended in 200 μl Binding buffer. The hybridization mixture was added to the bead solution gently and was subsequently incubated on a thermomixer at 850 rpm for 30 minutes at room temperature. To wash the beads, the supernatant was removed from beads on a Dynal magnetic separator and the beads were re-suspended in 500 μl Wash Buffer A (1×SSC/0.1% SDS), and incubated for 15 minutes at room temperature. Beads were then washed three times, each with 500 μl pre-warmed Wash Buffer B (0.1×SSC and 0.1% SDS) after incubation at 65° C. for 10 minutes. To elute captured DNA, beads were re-suspended in 50μ10.1M NaOH at RT for 10 minutes. The supernatant was transferred into a new 1.5 ml microcentrifuge tube after magnetic separation, and mixed with 50 μl Neutralizing Buffer (1M Tris-HCl, pH 7.5). DNA was purified with a Qiagen MinElute column and eluted in 17 μl of 70° C. EB buffer to obtain 15 μl of captured DNA library. The captured DNA library was amplified by Phusion Hot Start polymerase (New England Biolabs) using Illumina PE primer 1 and 2. The PCR program used was: 98° C. for 30 seconds; 6 ˜10 (depending on capture yield) cycles of 98° C. for 10 seconds, 65° C. for 30 seconds, 72° C. for 30 seconds; and a final incubation at 72° C. for 5 minutes. The PCR product was purified using GeneJET PCR Purification kit (ThermoFisher Scientific).

Real-Time PCR Assay

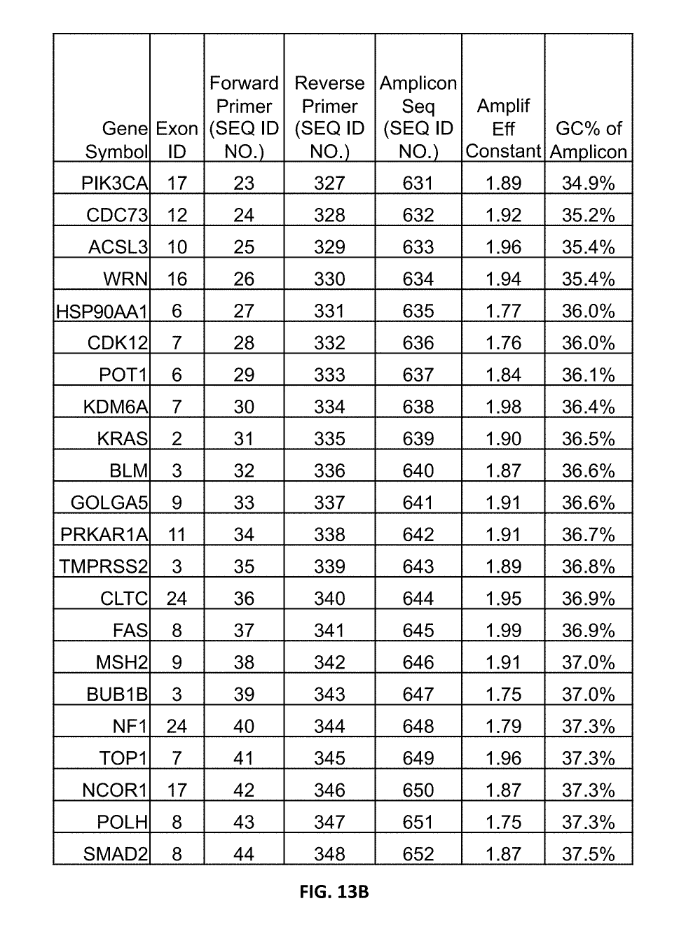

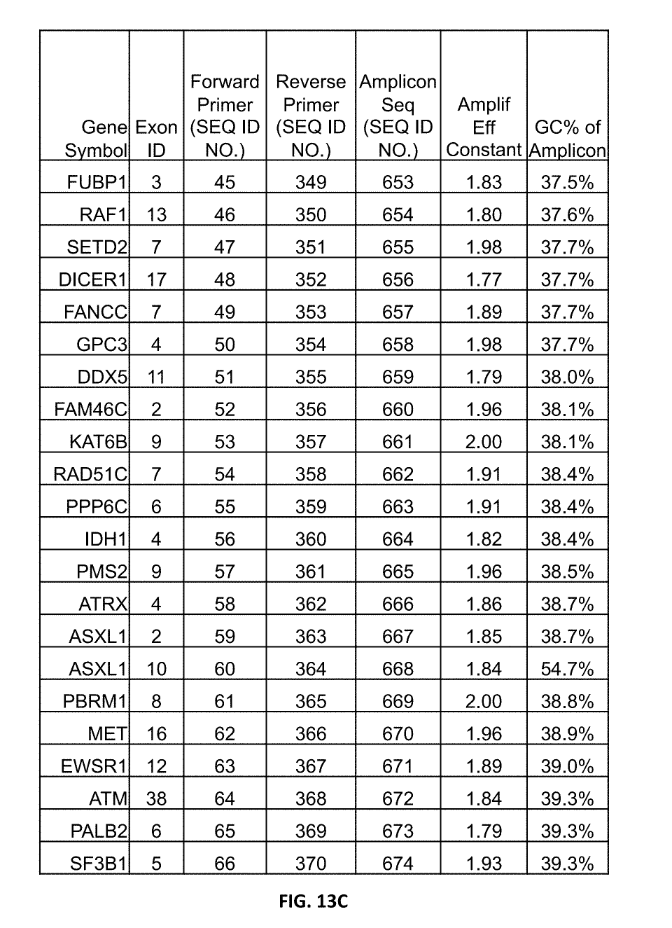

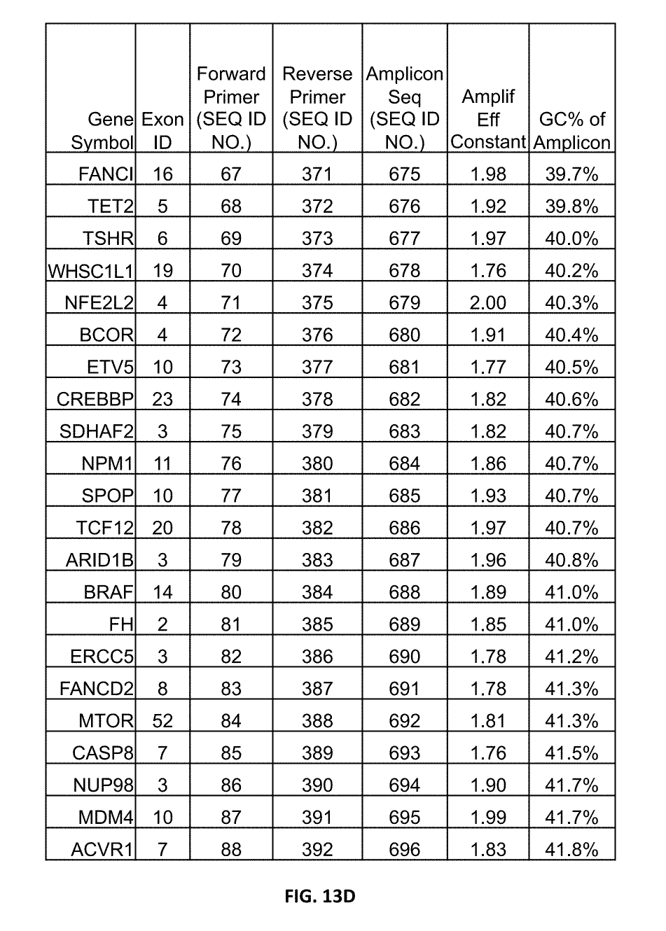

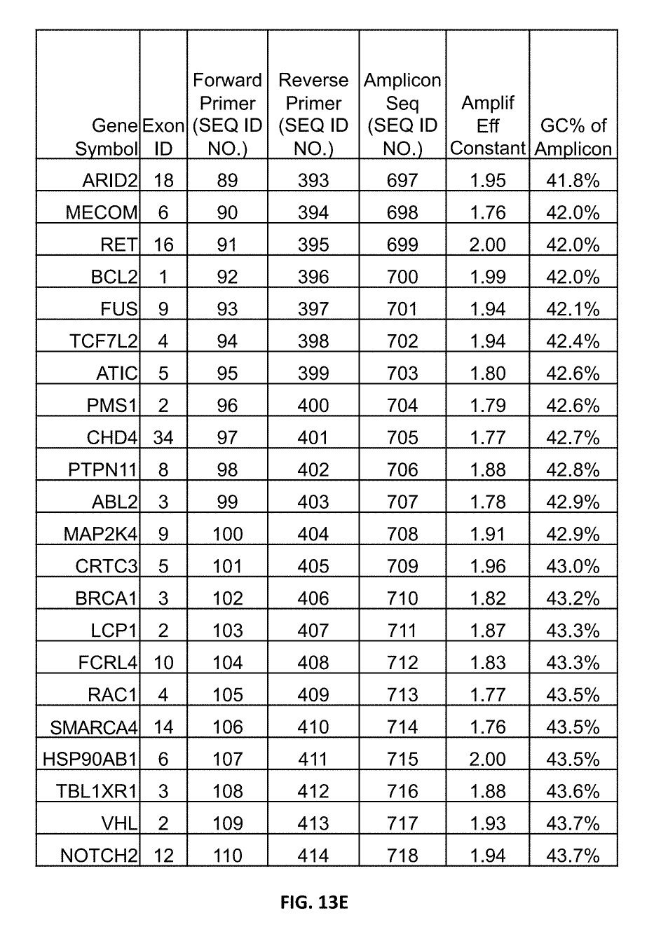

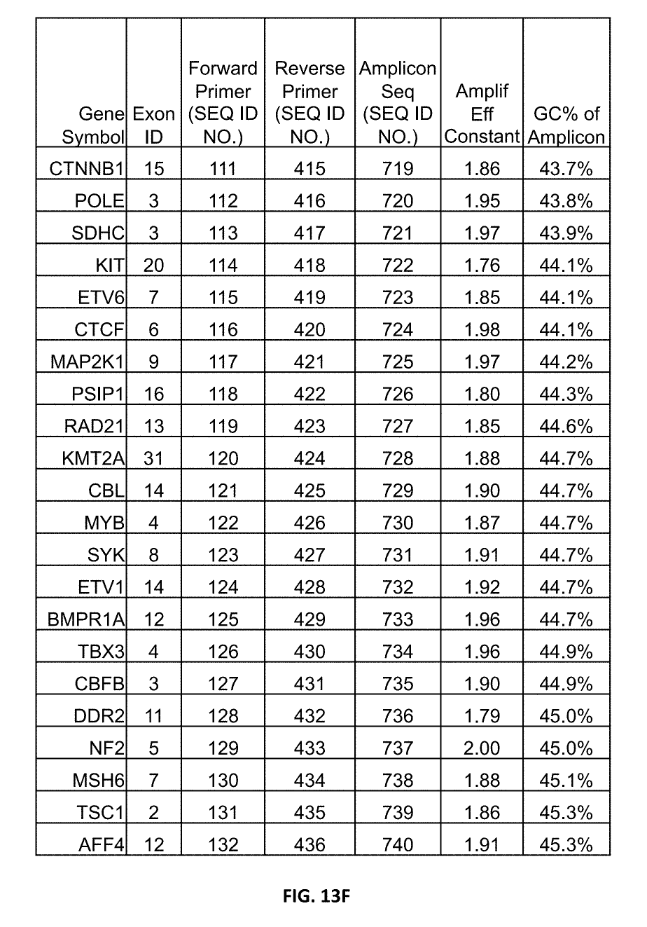

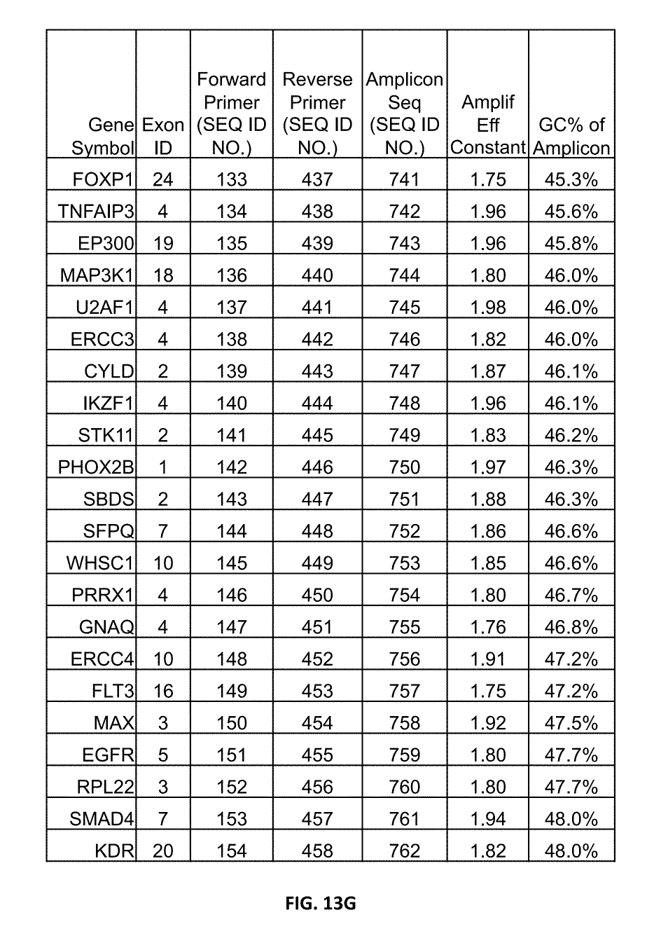

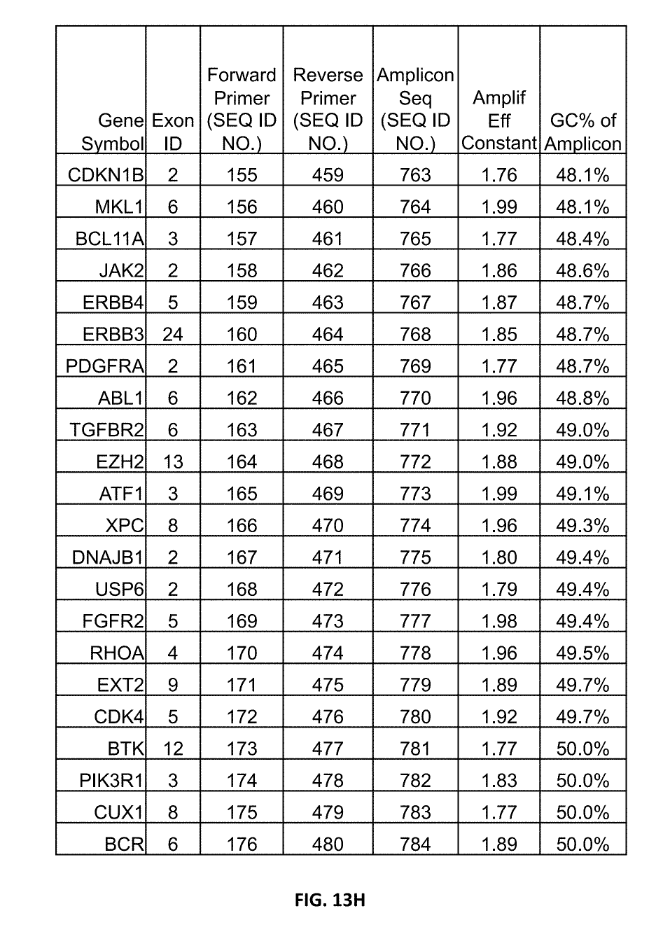

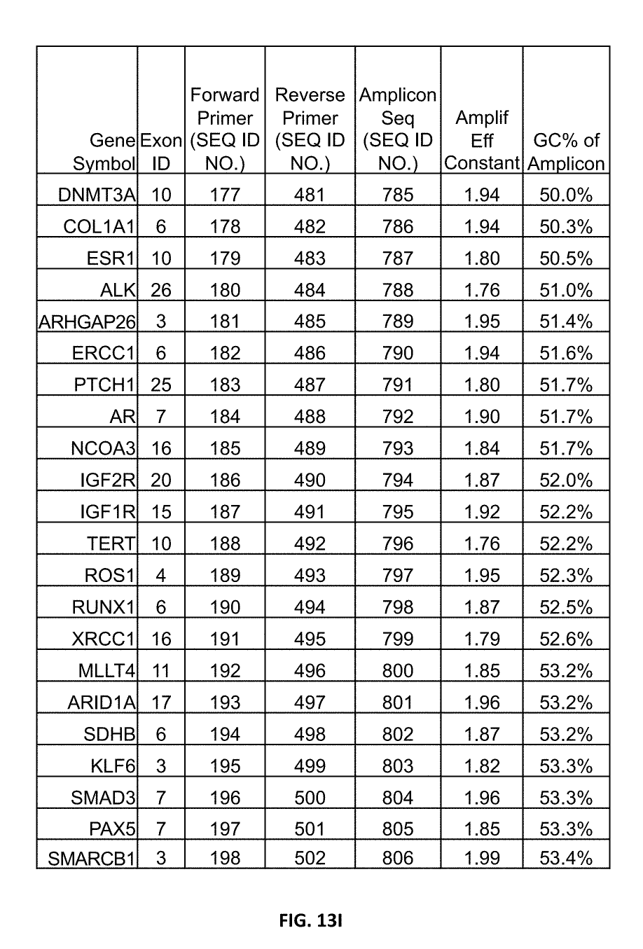

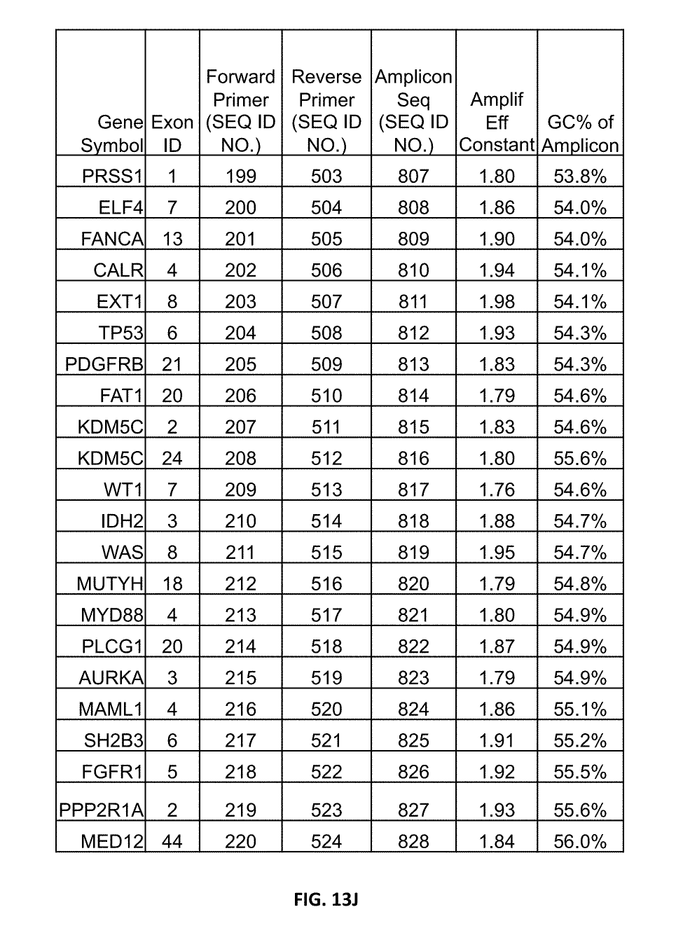

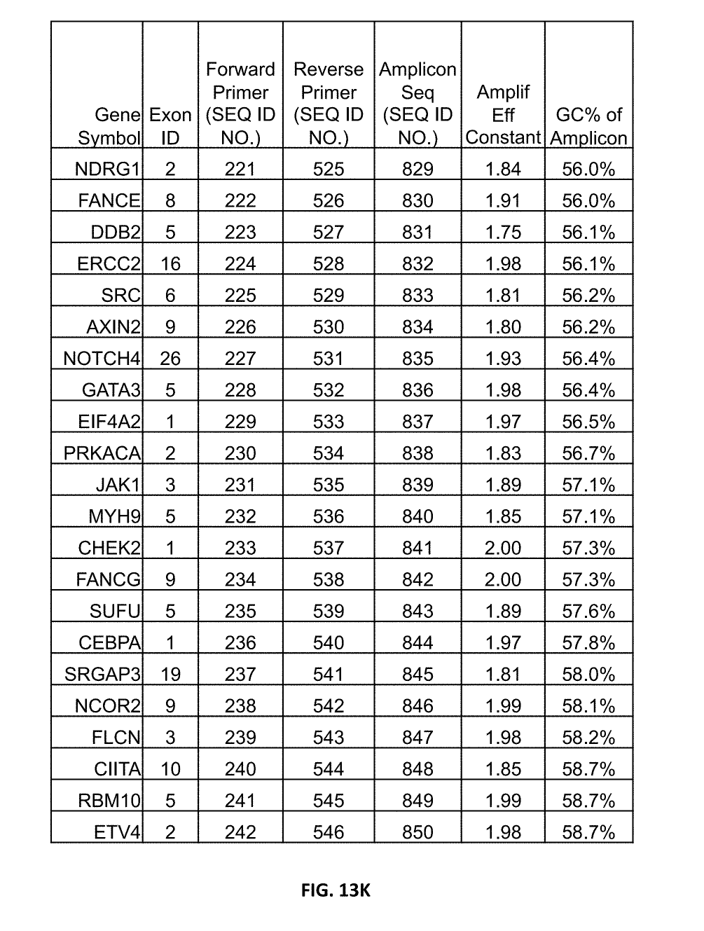

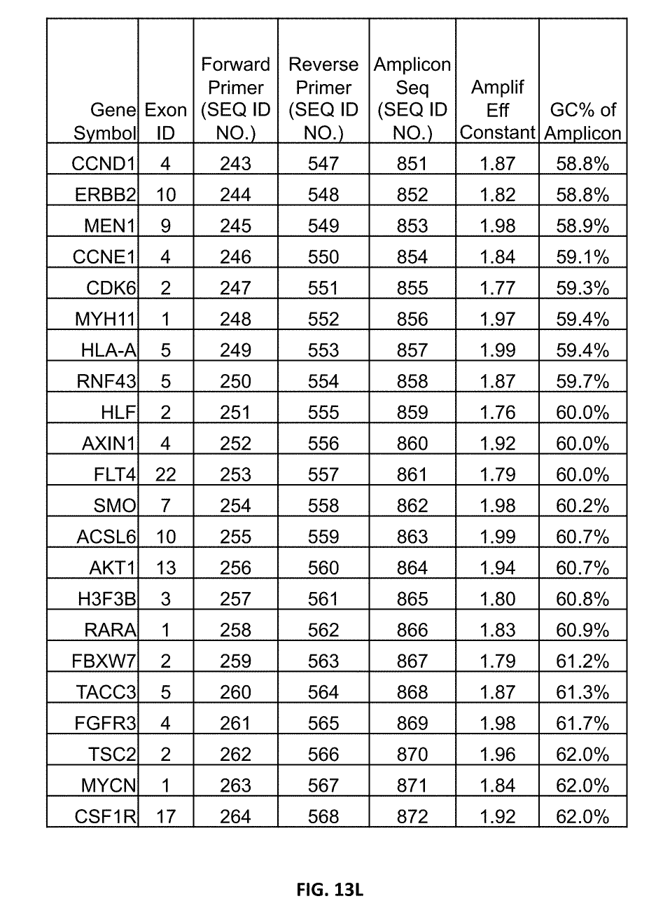

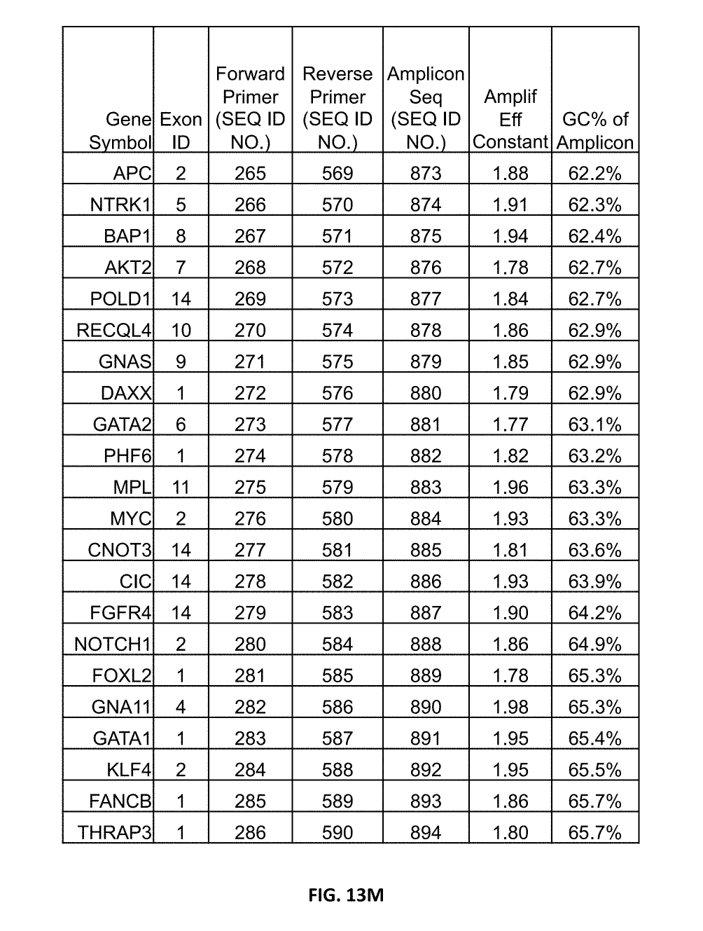

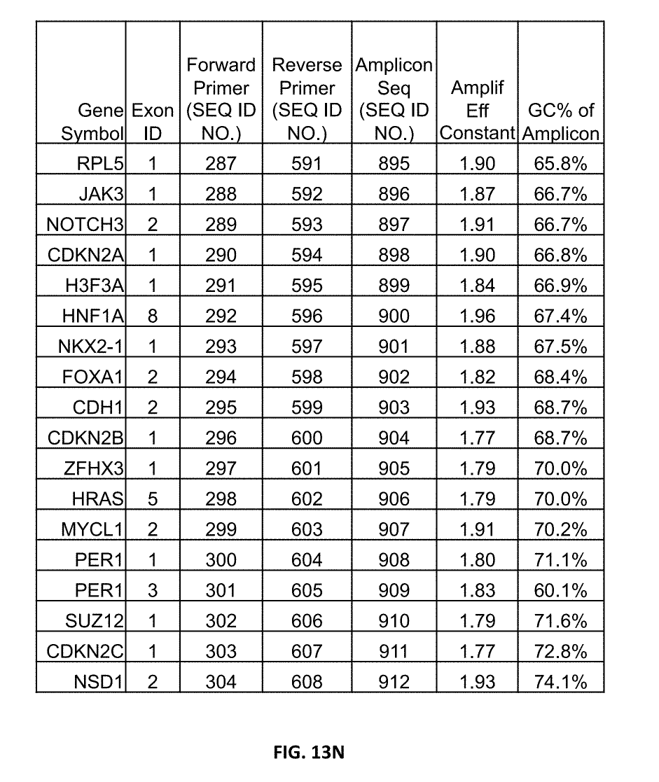

FIGS. 13A-13N illustrate 298 cancer-related gene targets, primer pairs, amplicon sequences, amplicon GC % and amplification efficiency constant for each real-time PCR detection are listed. Real-time PCR assays with SYBR green detection was carried out using an ABI PRISM 7500 Sequence Detection System (Applied Biosystems). Briefly, the reaction conditions consisted of 500 ng of genomic DNA or DNA library products, 0.2 μM primers, and SYBR Green Real-Time PCR Master Mix (ThermoFisher Scientific) in a final volume of 20 μl. Each cycle consisted of denaturation at 95° C. for 15 seconds, annealing at 58.5° C. for 5 seconds and extension at 72° C. for 20 seconds, respectively. Gene specific primers were designed using Primer 3 (Untergasser, Cutcutache et al. 2012) and their sequences are provided in FIG. 8. Reactions were run in triplicate in three independent experiments. The primer pair's standard amplification curve for each gene was established through using sequential dilutions of the “+” clone constructs containing the amplicon sequence. Amplification efficiencies for 298 target amplicons were established and listed in FIG. 8. Gene abundance ratios between different samples were calculated by the raising the gene specific amplification efficiency (AE) to the power of ΔCₜ value between different samples. For example, the ratio (r) of gene abundance in sample A vs sample B can be calculated through real-time PCR assay by: r_{(A/B)}=AE^{ΔCt}, ΔCₜ=C_{t(sample B)}−C_{t (sample A)}

Whole Exome Sequencing

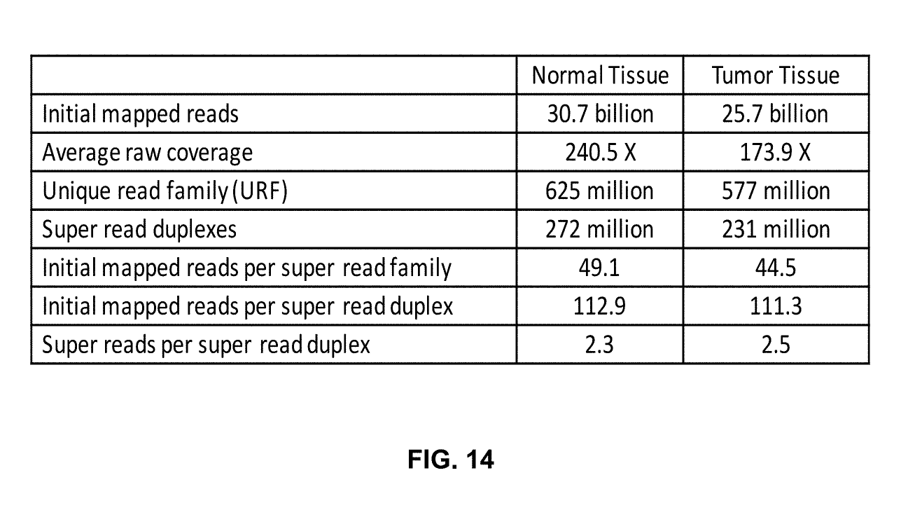

FIG. 14 show that initial mapped reads represent raw reads that contain the 12 nt barcode and mapped to the reference genome. Unique read family represents the number of URF. Each URF has a unique barcode and its sequence is obtained by consolidating read sequences arise from the same DNA molecule by PCR amplification. PCR errors are removed by requesting a sequence uniformity for over 95% of the reads within a URF. Super read duplexes represent the number of DNA duplex whose two strands are coming from two super reads.

Whole exome sequencing was performed on an Illumina HiSeq 2500 platform according to manufacturer's manual. Total numbers of on-target reads from randomly chosen 5 million to 50 million reads were calculated. After trimming and barcoded super read grouping, SNVs were called with GATK (version 3.6) in a default mode as recommended by the GATK documentation with reference genome of Hg19 (McKenna, Hanna et al. 2010). In brief, for every sample (tumor or normal DNA), sequencing result was preprocessed by mapping to reference genome with BWA (version 0.7.10), and duplicates were marked with Picard (version 2.0.1). Base Recalibration was performed to generate the reads ready for SNV analysis. For individually processed T/N pair reads, Indel Realignment was performed to generate pairwise-processed T/N pair reads. HaplotypeCaller was used for raw SNV calling. Output from variant calling was directly used for SNV detection by MuTect (version 1). Mutations were filtered through a 4-step approach introduced in the section “Mutation and ultra-rare mutation detection”. Low-quality variant with a Phred score <30.0 was abandoned. Paired SNVs from complementary reads bearing different barcodes were identified as true mutations and subject to further validation through Sanger sequencing. The data yields after each step of data analysis for an RNA probe-based DNA double strand capturing NGS study were shown in FIG. 14. SNVs identified and Sanger sequencing validation results were provided in FIGS. 15 A-E.

Mutation and Ultra-Rare Mutation Detection

The significantly increased number of unique reads obtained through RNA probe-based DNA double strand capturing enabled us to apply stringent filters with the following 4-step procedure.

Step 1) group reads with the same barcode that are representing PCR duplicates of an original barcoded single-stranded DNA molecule, and call it a unique read family (URF);

Step 2) combine reads within each URF obtained from Step 1) by requesting >95% sequence identity among the reads;

Step 3) extract the unique DNA sequence and the barcode sequence for each URF, and call it a “super read”

Step 4) for all the super reads identified in Step 3), find their paired complementary super reads, and only score sequence variants with matched complementary sequences from paired super reads. To accommodate damaged DNA molecules in the sample, complementary super reads may not be at the same length.

To evaluate the performance of RNA probe-based DNA double strand capture in detecting low frequency (ultra-rare) mutations, 100 ng tumor DNA sample was sequentially diluted by 10, 100, 1,000 and 10,000 folds, and spiked each of them into the same amount (100 ng) of genomic DNA extracted from the paired normal tissue of the aforementioned cancer patient. This design can simulate early stages of cancer occurrence, and represent the major obstacles in early cancer diagnostics using NGS, which is the very low allelic fractions of tumor specific mutations in the sample.

Build a highly accurate reference exome for ultra-rare mutation identification

To highly accurately assess the baseline mutation frequency of barcoded single-stranded library and RNA probe-based DNA double strand capturing pipeline, six replicates of standard NGS DNA libraries were constructed in parallel, each using 100 ng normal DNA input. These six replicates of exome datasets were used to re-build reference exome database for this particular patient by requesting that if the same SNV was observed in [symbol] 5 out of 6 independent datasets, the SNVs can be considered as germline variants and the reference exome sequence database will be updated. For a standard NGS pipeline, the error rate is 1%, and the chance to see the same random error at a fixed position for 5 times is (⅓×1%)⁵=4.12×10⁻¹³. This number means that if this approach is used to to sequence the whole human genome once, there is presumably going to be only one artificial error, because 3×10¹² human genome bases X (4.12×10⁻¹³)=1.24. However, only the human exome sequences are enriched and sequences, which is occupying only 1.5% of human genome, therefore the chance to see a single artificial error within the entire human exome is only 1.86% (=1.5% X 1.24). An updated highly accurate normal exome reference database of the patient was built accordingly.

### Example 2

RNA Probe-Based DNA Double Strand Capture Achieved a High Enrichment Efficiency

A method is developed to enrich targeted subgenomic sequences by RNA probe-based capture of both strands from the same DNA molecule, simultaneously. To assess the capture efficacy, RNA probes were created for the exome regions of the 298-gene panel adopted in this study. Real-time PCR assays were performed to detect and quantify the subgenomic regions of this gene panel in NGS library before and after RNA probe-based enrichment. No re-amplification of the library was performed after the capture to ensure that the amounts of DNA molecules obtained from RNA probe-based enrichment represent the captured yields for each gene.

FIG. 8A shows that for each of the six DNA libraries derived from different amounts (500 ng, 20 ng, 1 ng, 100 pg, 20 pg and 10 pg) of input genomic DNA, enrichment efficiency of 298 cancer-related genes were calculated. Recovery ratios of DNA single and double strand captures by RNA probes for all 298 genes in six libraries were quantified by real-time PCR assays detecting each gene's abundance in the libraries before and after single strand capture or double strand capture; FIG. 8B shows that an insert sequence composed of amplicon regions of five genes whose GC contents cover a broad range (27.3% to 74.1%) was cloned into a pcDNA vector; FIG. 8C shows that Real-time PCR analysis of sequential dilutions of the plasmid. 1, 10, 100, 1,000 and 10,000 femtomoles of the plasmids were added as templates for the assays. Cₜ value for each gene observed from different plasmid template amount was plotted, and trend lines were shown. No significant GC-dependent amplification bias was observed for real-time PCR assays; FIG. 8D shows that a whole genome library, and whole exome libraries captured by RNA based DNA single and double strand captures were analyzed on an agarose gel.

The same set of six libraries created from a sequentially diluted DNA input was adopted again. Such results indicate that each gene's capture ratio is in consistency for all six libraries (FIG. 8A). Such findings demonstrate that RNA probe-based DNA double strand capture efficiency for each gene is not dependent on the initial However, the capture ratios of different genes did vary to a significant extent (10.4% to 49.8%). Further investigation showed that RNA probe-based capture ratios for different genes were loosely correlated with the GC content of their amplicons (FIG. 8A). A very weak correlation (average R²=0.12) between amplicon GC contents and their RNA probe-based capture efficiencies were shown (FIG. 8A).

Library construction, RNA probe-based capture or the Real-time PCR assays are all potentially responsible for this GC content associated enrichment bias. Previous results have demonstrated that library construction is not significantly biased by GC content. Next, real-time PCR was investigated to check its potential GC content bias. 5 genes were chosen, PTEN, PALB2, ESR1, CSF1R, and NSD1, with distinct GC % in their amplicon sequences at 27.3%, 39.3%, 50.5%, 62%, and 74.1%, respectively (FIGS. 13A-13N). A plasmid (pcDNA 6.2 vector) containing a DNA insert composed of all five genes' amplicon regions separated by a 100 bp flanking sequence is cloned (FIG. 8B) and sequentially diluted to simulate variable amounts of the gene fragments after capture. All 5 gene fragments were cloned into the same plasmid to ensure the equal abundance between different genes in every diluted sample. Real-time PCR was performed to detect the copy number of plasmids by detecting each of the five genes in a series of diluted plasmid samples. As shown in FIG. 8C, there is no obvious GC-content-associated bias observed from the real-time PCR amplifications of all five genes using these primers. The 298 pairs of primers adopted in real-time PCR assays were designed with their Tₘ values all falling into a narrow range of 57° C. to 61° C. This restriction of Tₘ and short amplicon sequences with similar lengths (˜around 150 bp) helped ensure the uniformity of real-time PCR assays for all genes (FIGS. 13A-13N). Therefore, the only possible step, where the GC content related capture ratio bias is created, should be the RNA probe-based capture itself. Enrichment bias for hybridization-based subgenomic capture has been reported to be owing to GC content. Whole exome capture NGS studies were conducted to assess further the impact of GC content to the RNA probe-based capture efficiency of target subgenomic regions.

It is important to note that in this RNA probe-based capture, complementary RNA probes were used to capture both DNA strands of the target regions, and attempts were made to assess if there is any capture efficiency difference between using only one set of RNA probes to capture only one strand of target DNA and using two sets of RNA probes to capture both strands of target DNA simultaneously. These two capture methods were performed in parallel with two equal aliquots (500 ng) of DNA libraries, where each library was created from the same 20 ng genomic DNA. A whole genome library, captured yields by RNA probes targeting both strands of DNA molecules or RNA probes targeting only a single strand of the DNA target molecule were analyzed on an agarose gel (FIG. 8D). Real-time PCRs for the 298-gene panel were performed to evaluate the capture ratios of RNA probe single strand capture and double strand capture for all the genes (FIG. 8A). The average capture ratios for the target nucleic acid sequence capture method as disclosed herein (i.e. the double strand-targeting RNA probe-based capture method) is 29.2% across all genes in different libraries, much higher than the ratios observed from the conventional single strand-targeting RNA probe-based capture approach (˜8.5% on average). These results have demonstrated that to capture target DNA sequences by complementary RNA probes through hybridizing to both strands of the DNA duplex molecule simultaneously achieved an over 3-fold increase in capture efficiency compared to capturing a DNA single strand alone through RNA probes.

### Example 3

Whole Exome Sequencing

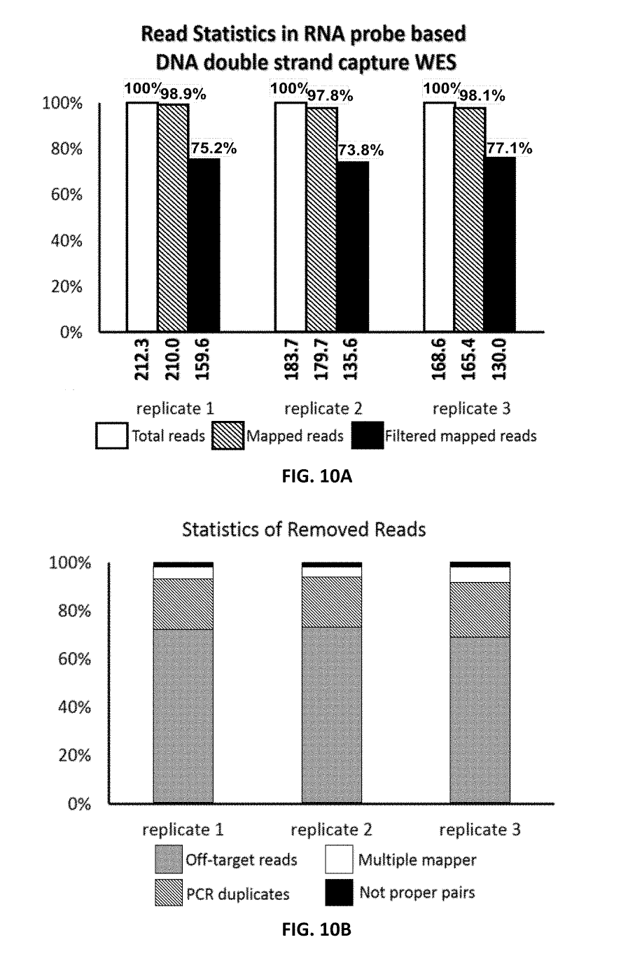

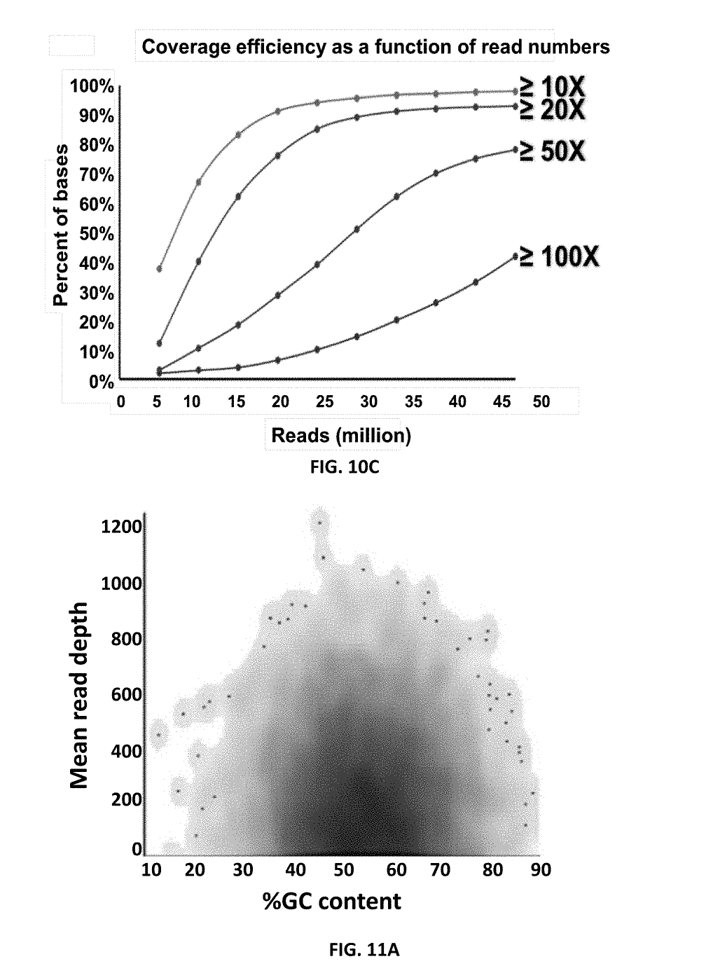

FIG. 10A shows a bar plot of percentage of initial reads, mapped reads and reads remained after filtering. Results were obtained from three technical replicates. Numbers of reads were shown under each bar with the unit of 1 million reads. FIG. 10B shows a stacked bar plot of subgroups of filtered reads in triple replicates. FIG. 10C shows a coverage efficiency correlation with read numbers. The percentage of target bases covered at [symbol] 10×, [symbol] 20×, [symbol] 50× and [symbol] 100× depths with 5 million to 50 million reads were shown.

To evaluate the performance of RNA probe-based DNA double strand capture in NGS, WES assays were performed using this method and compared the data to what obtained through standard NGS library preparation with a standard exome enrichment procedure. All libraries were constructed with 100 ng genomic DNA derived from the normal tissue of the cancer patient, and three technical replicates were performed for each sample. All NGS runs were carried out on the same Illumina HiSeq 2500 platform with the same technical specifications of the runs. As shown in FIG. 10A, an average of 188 million reads were obtained from RNA probe-based DNA double strand capturing WES, where 98.3% were aligned to the human genome, and the total read counts were significantly more (1.6 folds) than that from the standard sequencing pipeline. The higher numbers of reads for the libraries presumably came from the ultra-sensitive single-stranded DNA library construction, and the much more efficient RNA probe-based enrichment designed to capture both DNA strands (including DNA molecules that have damages ranging from minor single strand breaks to major damages on both strands).

All NGS data were analyzed on the same software pipeline with the same settings. Raw reads were filtered to remove duplicates, multiple mappers, improper pairs, and off-target reads. On average 75.4% reads were retained after filtering (FIG. 10A). For the reads that were removed, 71.8% were off-target reads, which were mapped to the human genome but outside of the target regions, 21.6% were PCR duplicates, and the remaining reads were mapped to multiple sites of the genome or not mapped at all (FIG. 10B). No statistically significant difference was observed in all the specifications measures for the three technical replicates in this experiment, which indicates that library construction and RNA probe-based DNA double strand capturing pipeline is technically highly reproducible (FIGS. 10A, and 10B).

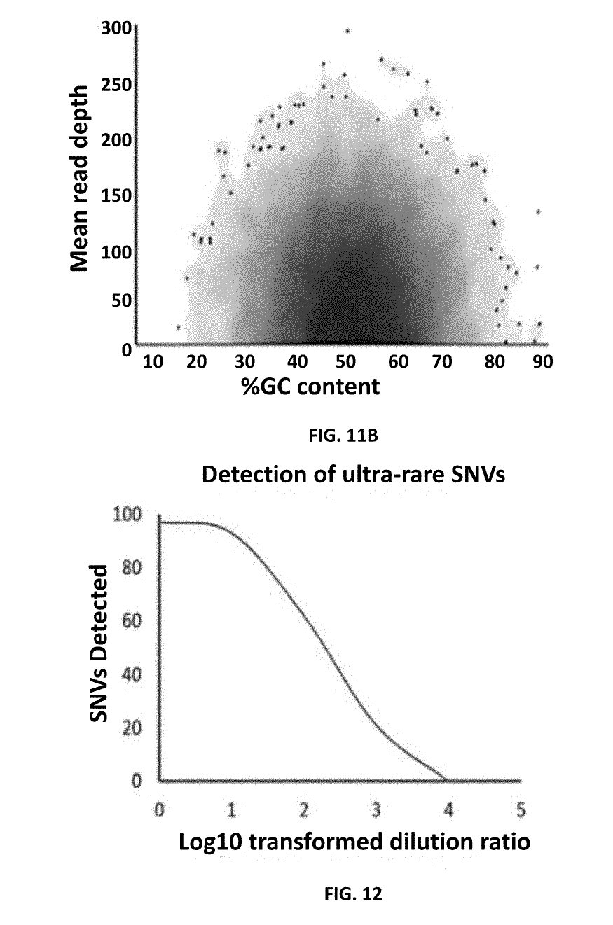

Next, the correlation between coverage efficiency and sequencing depth in NGS library with RNA probe-based DNA double strand capturing was evaluated. Filtered reads were randomly selected in 5 million read increments from 5 million to 50 million. The fractions of the retained on-target reads covering the depths of at least 10×, 20×, 50×, and 100× were plotted using randomly selected 5 to 50 million reads (FIG. 10C). 20 million reads could cover close to 90% of the target bases with no less than 10× depth. With 50 million reads, over 90% target bases were covered by at least 20×. The efficiency of coverage is not only dependent on the efficiency of library construction but also dependent on the length of the sheared molecules that were initially incorporated into the pipeline. For the current study, the average length of sheared DNA molecule is 150 bp. These real-time PCR results for the 298-gene panel indicated that enrichment efficiency of the library construction approach is not significantly biased by GC content (FIG. 8). Density plots were created to show GC content against normalized mean read depth for RNA probe-based DNA double strand capture WES study with normal tissue DNA (FIG. 11A), and DNA library WGS study with normal tissue DNA (without enrichment for whole exome, FIG. 11B).

To assess the impact of GC content on WES result, normalized mean read depth against GC content was plotted. There is a correlation between GC content and read depth in the WES experiment (FIG. 11A), and this bias is reduced in a WGS study (FIG. 11B). In this method, the mean read depth ratios of GC50%/GC20%=1.55, which is significantly lower than the ratio of 2.0 reported by numerous studies (Benjamini and Speed 2012, Meienberg, Zerjavic et al. 2015), which demonstrates a lower GC bias in this method.

### Example 4

Detection of SNVs

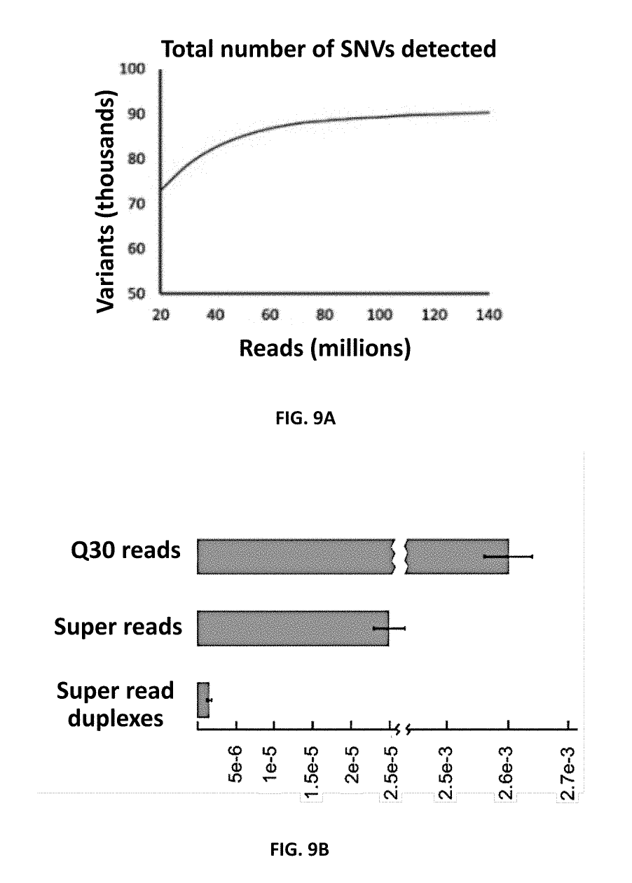

FIG. 9A shows that total number of SNVs detected at increasing read count thresholds. Sensitivity increases at higher read counts but quickly reaches a plateau with more than 80 million reads. FIG. 9B shows average SNV frequencies of normal tissue DNA measured by three approaches: a standard NGS approach where barcodes were directly trimmed off, a super read based approach by barcoded single-stranded library based NGS without matching variants from both DNA strands (without the last step of the 4-step procedure), and a super read approach by barcoded single-stranded library based NGS matching the SNV on both strands (all steps in the 4-step procedure were performed). All three approaches were performed with RNA probe-based DNA double strand capture WES.

One of the most important goals of exome sequencing is to identify sequence variants that are disease-causing or of clinical significance. To evaluate the sensitivity and specificity of sequence variant identification performance of library construction and RNA probe-based DNA double strand capturing, a WES study was conducted with 100 ng genomic DNA from a pair of normal and tumor tissue samples obtained from the same cancer patient. The same SNV calling pipeline was used for all data analysis in this study. Briefly, the normal DNA libraries created by library construction and RNA probe-based DNA double strand capturing method was sequenced and the data were analyzed using a standard data analysis pipeline, where the single-stranded barcodes were directly trimmed off, and 78,721 SNVs were detected from the exonic sequences of normal DNA sample at a read count of 30 million (error frequency 2.6×10⁻³, FIG. 9A). The total number of SNVs detected from 30 million reads of the normal tissue DNA is significantly higher than what was reported on other platforms (Clark, Chen et al. 2011). Next, further investigation was made to check if there is any bias in SNVs identified using the standard NGS data analysis workflow. Transition-transversion (ts/tv) ratio is routinely used to evaluate the specificity of new SNP calls. The ts/tv ratio on the target regions of WES was calculated to be 2.766, higher than the reported ts/tv ratios of 2.0-2.1 for WGS data. The ts/tv ratio in CCDS exonic regions as was then determined as 3.225, which falls into the range of 3.0˜3.3 for reported exonic variations. The reason for RNA probe-based DNA double strand capture for whole exome sequencing to have a higher ts/tv ratio than reported WGS studies is because target regions of sequencing are enriched for exons, and only contain UTRs and short flanking sequences within introns.

The accuracy of mutations enriched by DNA based mutation calling was then examined. Following the 4-step data analysis procedure introduced in Materials and Methods, super reads were generated after Step 3). Steps 1˜3 helped to reduce the mutation frequency by over two orders of magnitude from 2.6×10⁻³ down to 2.5×10⁻⁵ by removing most PCR related errors (FIG. 9B). This result indicates that PCR related artificial mutations dramatically reduce NGS sequencing accuracy. To detect rare mutations, or even ultra-rare mutations using NGS, a correction for PCR errors is mandatory. As outlined in Step 4), attempts were then made to further reduce artificial errors of mutation calling by using the redundant sequence information offered by complementary DNA strands that were originally from the same DNA duplex molecule. These results indicated that such procedure resulted in a single base mutation frequency of 1.6×10⁻⁶ (FIG. 9B). For any single base in the DNA sequences, the possibility of having the same artificial error on a paired position is ⅓× (2.5×10⁻⁵)²=2.08×10⁻¹⁹, which is equivalent to one artificial error per 4.8×10⁹ nucleotides. This is the theoretical error rate for the pipeline. The total amount of DNA sequence data and the remaining amount of data after each step can be found in FIG. 14, where a stepwise drop of data amount is correlated to the increase of mutation calling stringency.

To determine the accuracy of variant detection by library construction and RNA probe-based DNA double strand capturing for clinically relevant mutations, the WES data generated from the normal and tumor tissue pair were analyzed side-by-side. For all assessed heterozygous exonic positions, the result was filtered through such 4-step procedure. The filtered result showed that for RNA probe-based DNA double strand capturing, WES study identified 97 sequence variants that were exclusively detected in tumor tissue DNA sample with [symbol] 100× coverage at different fractions. 40 moderate- to high-abundance (>5%) variants were subject to Sanger sequencing validation, and 38 were confirmed (FIGS. 15A-15E). Two variants failed to be validated where both allelic fractions were low and beyond the detection limit of Sanger sequencing. 57 sequence variants (with mutant allele fractions <5%) were not subject to Sanger sequencing validation at all, due to the limited sensitivity of Sanger sequencing (Tsiatis, Norris-Kirby et al. 2010).

### Example 5

A Protocol for RNA Probe-Based DNA Double Strand Capture

Production of the RNA Probes

The entire exome sequences for every human gene by sequence synthesis and molecular cloning based on Hg19 reference human genome sequences. In brief, exome sequences in 32,524 CCDS IDs containing −50 bp and +50 bp intronic sequences were cloned into pcDNA 6.2 vector. The total DNA sequences used to generate RNA probes cover a 72.6 Mb genome region, where all the exomes with their −50 bp and +50 bp flanking intronic sequences, as well as 5′ and 3′ UTRs for each gene were included.

Two clones for each target sequence were constructed, where a T7 promoter was inserted at the 5′ end of the plus strand in the “+” clone and the 5′ end of the minus strand in the “−” clone (FIG. 4B and FIGS. 7A and 7B).

Two pools of clones were established for any give number of genes following the rule that the two clones for the same DNA sequence are separated into two systems, where one system (FIG. 4B and FIGS. 7A and 7B, left panel, “+” Clone) produced the RNA probes targeting the plus strand of the DNA target, and the other system (FIG. 4B and FIGS. 7A and 7B, right panel “−” Clone) produced the RNA probes targeting the minus strand of the DNA target through in vitro transcription.

AmpliScribe™ T7 Flash™ Biotin-RNA Transcription kit is used for RNA probe production and amplification

Prepare the mix as following:

| | volume per |
|---|---|
| component | reaction (μl) |
| Plasmid library with T7 promoter(100 ng) | 5.5 |
| T7 flash buffer 10X | 2 |
| NTP/biotin-UTP premix | 8 |
| 100 mM DTT | 2 |
| RNase Inhibitor | 0.5 |
| AmpliScribeT7 Flash enzyme | 2 |
| Total | 20 |

Mix well and incubate at 30° C. for 4 hours.

Add 1 μl DNaseI. Incubate at 37° C. for 15 minutes.

Purify the RNA probes using 2×RNA AMPure beads. Elute into 80 μl. You should have 150 μg probe now.

sonication of the RNA products to generate 100-150 nt RNA fragments as probes

Turn on BioRuptor and water bath (set to 3° C.) at least 45 minutes before starting.

Place up to 1 μg of RNA adjusted to 57 μl with 1×TE buffer in a BioRuptor microtube.

Shear with below setting for a target size range of 100-150 nt:

| Setting | value |
|---|---|
| Intensity | H |
| On:Off | 30:30 |
| Cycles | 35 |

Hybridize the probes with the DNA library

Mix the following components as DNA library+block mix at room temperature:
4.3 μl of DNA library (150 ng/ul)
3 μl of Human Cot-1 DNA (Life Technologies 15279-101)
3 μl of Salmon sperm (Life Technologies 15632-011)
0.7 μl Customized blocking oligos mix (1000 μM), sequence shown below:
Blocking Oligo 1 (as set forth in SEQ ID NO. 913)
Blocking Oligo 2 (as set forth in SEQ ID NO. 914)

Mix well by pipetting.

Transfer the DNA-library+block mix to 384-well PCR plate. Seal the plate will microAmp clear adhesive film (cat#4306311 from ABI) for tight sealing.

Centrifuge the plate briefly to collect the liquid at the bottom of the well.

Run the following thermocycler program (with 105° C. heated lid):

95° C. for 5 minutes;

67.5° C. forever

Prepare the Hybridization Buffer immediately after putting DNA-library+block mix in the thermocycler. Mix the following components at room temperature to prepare the Hybridization

Buffer:

| | volume in 1 |
|---|---|
| component | reaction (μl) |
| 20 × SSPE | 12.5 |
| 0.5M EDTA | 0.5 |
| 50 × Denhardt's | 5 |
| 10% SDS | 6.5 |
| Total | 24.5 |

With the 384-well plate in the thermocycler at 67.5° C., transfer 24.5 μl of Hybridization Buffer into a new well of the plate, seal the plate with adhesive film.

Incubate the Hybridization Buffer at 67.5° C. for at least 5 minutes (could be longer) while the RNA-Probe Library get prepared.

Prepare the RNA probes immediately after putting the Hybridization Buffer in the thermocycler. Mix the following components on ice to prepare the RNA probes:

| | volume in 1 |
|---|---|
| component | reaction (μl) |
| RNA probes from “+” clone transcripts (800 ng/μl) | 2.5 |
| RNA probes from “−” clone transcripts (800 ng/μl) | 2.5 |
| RNase Inhibitor (20 U/μl) | 0.5 |
| Nuclease-free water | 1.5 |
| Total | 7 |

With the 384-well plate in the thermocycler at 67.5° C., transfer 7 μl of RNA probes into a new well of the plate, seal the plate with adhesive film.

Incubate the RNA probes at 75.5° C. for 2 minutes with the heated lid.

Open the lid and maintain the plate at 75.5° C. Take 13 μl of pre-heated Hybridization Buffer and add it to the RNA probes.

Transfer 10 μl of DNA-library+block mix to the RNA probes.

Mix well by slowly pipetting up and down several times. The hybridization mixture should be ˜30 μl.

Seal the well with double adhesive film.

Incubate the hybridization mixture for 12˜48 hours at 67.5° C. with heated lid at 105° C.

Extracting the hybridized RNA-DNA molecules

Wash 50 μl Dynal MyOne Streptavidin C1 magnetic beads (ThermoFisher Scientific) with 200 μl Binding buffer (1M NaCl, 10 mM Tris-HCl, pH 7.5, and 1 mM EDTA) three times in 1.5 ml microfuge tube and resuspend in 200 μl binding buffer.

Add the entire hybridization mixture directly from the thermocycler to the bead solution, and invert the tube to mix several times.

Rotate the hybridization mixture/bead solution 360 deg. for 15 minutes at room temperature.

After binding, the beads are separated from the solution on a Dynal magnetic separator and the supernatant is removed.

Washing Procedure

Wash the beads for 15 minutes at room temperate in 500 μl wash buffer I (1×SSC/0.1% SDS).

Wash the beads for 15 minutes at 67.5° C. on a heating block with shaking, three times in 500 μl 67.5° C. pre-heated wash buffer II (0.1×SSC/0.1% SDS).

Mix the beads in 50 μl 0.1M NaOH at room temperature for 10 minutes.

Transfer the supernatant to a new tube.

Add 50 μl of Neutralizing buffer (1M Tris-HCl, pH 7.5).

Neutralized DNA is desalted and concentrated by AMPure beads with ratio 1:1 (beads:sample), elute in 20 μl 1×TE buffer.

Post-Capture amplification

PCR mix contains: (per reaction)

| | | |
|---|---|---|
| Captured DNA | 20 | ul |
| Water | 65 | ul |
| DMSO | 2.5 | ul |
| 5X Phusion Buffer | 10 | ul |
| 10 mM dNTPs | 1 | ul |
| Index PE primer II | 0.25 | ul |
| PE primer I | 0.25 | ul |
| HotStart Phusion | 1 | ul |

mix well

Amplification conditions:

| | | | |
|---|---|---|---|
| Step 1: 1 cycle | 98° C. | 1 | minute |
| Step 2: 14 cycles of | 98° C. | 10 | seconds |
| | 65° C. | 30 | seconds |
| | 72° C. | 30 | seconds |
| Step 3: 1 cycle | 72° C. | 5 | minutes |

| | | |
|---|---|---|
| Step 4: 1 cycle | 4° C. | hold |

The PCR is done in two wells for each sample, 50 ul each. Then the amplified PCR product was purified using AMPure beads with ratio 1:1 (beads:sample), elute in 30 μl 1×TE buffer.

Use Qubit to quantify yield. You will have ˜20 ng/ul in general.

### REFERENCE

Benjamini, Y. and T. P. Speed (2012). “Summarizing and correcting the GC content bias in high-throughput sequencing.” Nucleic Acids Res 40(10): e72.
Clark, M. J., et al. (2011). “Performance comparison of exome DNA sequencing technologies.” Nat Biotechnol 29(10): 908-914.
McKenna, A., et al. (2010). “The Genome Analysis Toolkit: a MapReduce framework for analyzing next-generation DNA sequencing data.” Genome Res 20(9): 1297-1303.
Meienberg, J., et al. (2015). “New insights into the performance of human whole-exome capture platforms.” Nucleic Acids Res 43(11): e76.
Tsiatis, A. C., et al. (2010). “Comparison of Sanger sequencing, pyrosequencing, and melting curve analysis for the detection of KRAS mutations: diagnostic and clinical implications.” J Mol Diagn 12(4): 425-432.
Untergasser, A., et al. (2012). “Primer3—new capabilities and interfaces.” Nucleic Acids Res 40(15): e115.
Van Allen, E. M., et al. (2014). “Whole-exome sequencing and clinical interpretation of formalin-fixed, paraffin-embedded tumor samples to guide precision cancer medicine.” Nat Med 20(6): 682-688.

1. A kit for enriching at least one target nucleic acid sequence from a biological sample, comprising:
at least one pair of RNA probe sets, each pair comprising a first RNA probe set and a second RNA probe set configured to respectively target two antiparallel strands of a duplex segment in each of the at least one target nucleic acid sequence, wherein each RNA probe in any of the first RNA probe set and the second RNA probe set is labelled with an immobilization portion; and
a solid support labelled with a coupling partner on a surface thereof, wherein the coupling partner is configured to be able to form a secure coupling to the immobilization portion to thereby allow immobilization of each RNA probe in any of the first RNA probe set and the second RNA probe set in the each of the at least one pair of RNA probe sets onto the solid support.

2. The kit of claim 1, wherein each RNA probe in any of the first RNA probe set and the second RNA probe set in the each of the at least one pair of RNA probe sets has a length of about 100-150 nt,

3. The kit of claim 1, wherein the secure coupling between the immobilization portion and the coupling partner labelled onto the solid support comprises at least one of a stable non-covalent binding or a covalent connection.

4. The kit of claim 3, wherein the secure coupling comprises a stable non-covalent binding, wherein the immobilization portion comprises a biotin moiety, and the coupling partner labelled onto the solid support at least one of streptavidin, avidin, or an anti-biotin antibody.

5. The kit of claim 1, wherein the solid support comprises at least one of a magnetic bead, a filter, a resin bead, a nanosphere, a plastic surface, a microtiter plate, a glass surface, a slide, a membrane, a microfluidic channel, a chip or a matrix.

6. The kit of claim 1, further comprising an apparatus having a working surface as the solid support, wherein the first RNA probe set and the second RNA probe set in each pair are respectively conjugated onto the working surface, arranged such that each RNA probe in the solid support-conjugated first RNA probe set does not substantially interact with each RNA probe in the solid support-conjugated second RNA probe set.

7. The kit of claim 6, wherein the apparatus is one of a column, a microfluidic channel, or a chip.

8. The kit of claim 6, wherein the solid support-conjugated first RNA probe set and the solid support-conjugated second RNA probe set in each pair are respectively arranged at at least one pair of two different regions of the working surface of the apparatus.

9. The kit of claim 8, wherein the solid support-conjugated first RNA probe set and the solid support-conjugated second RNA probe set in each pair are alternately arranged at more than one pairs of two different regions of the working surface of the apparatus.

10. The kit of claim 6, wherein the solid support-conjugated first RNA probe set and the solid support-conjugated second RNA probe set in each pair are mixedly arranged on the working surface of the apparatus, configured such that each RNA probe from the first RNA probe set has a relatively large distance to each RNA probe from the second RNA probe set to thereby substantially prevent an interaction therebetween.

11. The kit of claim 6, wherein the apparatus is configured to allow the biological sample to flow through the working surface for more than one round.

12. The kit of claim 1, wherein one or more of the at least one target nucleic acid sequence are each in a polynucleotide comprising at least one un-targeted sequence, wherein the kit further comprises:
at least one blocking oligo, configured to respectively hybridize with, and to thereby block, at least one strand of each of the at least one un-targeted sequence in the polynucleotide.

13. The kit of claim 12, wherein the at least one un-targeted sequence in the polynucleotide comprises a first adaptor sequence and a second adaptor sequence flanking each of the one or more of the at least one target nucleic acid sequence, wherein:
the at least one blocking oligo is configured to respectively block one strand of the first adaptor sequence and one strand of the second adaptor sequence in the polynucleotide.

14. A method for enriching at least one target nucleic acid sequence from a biological sample utilizing the kit according to claim 1, comprising:
preparing at least one pair of RNA probe sets, each pair comprising a first RNA probe set and a second RNA probe set configured to respectively target two antiparallel strands of a duplex segment in each of the at least one target nucleic acid sequence, wherein each RNA probe in any of the first RNA probe set and the second RNA probe set is labelled with an immobilization portion configured to allow immobilization onto a solid support; and
capturing each strand of the at least one target nucleic acid sequence from the biological sample through hybridization of both the first RNA probe set and the second RNA probe set in the each of the at least one pair of RNA probe sets respectively with the two antiparallel strands of the duplex segment in the each of the at least one target nucleic acid sequence and through immobilization onto the solid support via the immobilization portion labelled onto each RNA probe in any of the first RNA probe set and the second RNA probe set.

15. The method of claim 14, wherein one or more of the at least one pair of RNA probe sets is prepared through chemical synthesis, and the preparing at least one pair of RNA probe sets comprises:
performing chemical synthesis reactions to thereby obtain the one or more of the at least one pair of RNA probe sets.

16. The method of claim 14, wherein one or more of the at least one pair of RNA probe sets is prepared through transcription, and the preparing at least one pair of RNA probe sets comprises:
performing transcription reactions to thereby obtain the one or more of the at least one pair of RNA probe sets.

17. The method of claim 16, wherein the performing transcription reactions to thereby obtain the one or more of the at least one pair of RNA probe sets comprises:
performing transcription reactions such that each RNA probe in any of the one or more of the at least one pair of RNA probe sets is labelled with the immobilization portion during each of the transcription reactions.

18. The method of claim 17, wherein each of the transcription reactions is performed in presence of NTPs labelled with the immobilization portion, where the NTPs comprises at least one of ATPs, UTPs, GTPs, and CTPs.

19. The method of claim 18, wherein the NTPs labelled with the immobilization portion comprise biotin-labelled UTPs having a relative molar percentage of 2%-100% in all UTPs present in each of the transcription reactions.

20. The method of claim 16, wherein each RNA probe in any of the one or more of the at least one pair of RNA probe sets is labelled with the immobilization portion after each of the transcription reactions, and the performing transcription reactions to thereby obtain the one or more of the at least one pair of RNA probe sets comprises:
performing the transcription reactions; and
performing a labelling.

21. The method of claim 16, wherein the performing the transcription reactions comprises:
providing a plurality of DNA vectors, comprising at least one pair of DNA vectors, each pair comprising a first DNA vector and a second DNA vector configured to respectively allow transcription of a first RNA molecule and a second RNA molecule respectively targeting two antiparallel strands of a duplex segment in each of the one or more of the at least one target nucleic acid sequence; and
performing the transcription reactions over the plurality of DNA vectors.

22. The method of claim 21, wherein each of the plurality of DNA vectors comprises a promoter, selected from one of a T3 promoter, a T7 promoter, or a SP6 promoter.

23. The method of claim 21, wherein in the performing the transcription reactions over the plurality of DNA vectors, at least one of the transcription reactions is performed in vitro or in vivo.

24. The method of claim 21, wherein the performing the transcription reactions over the plurality of DNA vectors comprises:
pooling the plurality of DNA vectors to obtain at least two DNA vector pools, such that the first DNA vector and the second DNA vector in the each pair of DNA vectors are not in a same DNA vector pool; and
performing a transcription reaction over each of the at least two DNA vector pools respectively to obtain RNA molecules corresponding to the each of the at least two DNA vector pools.

25. The method of claim 24, further comprising, after the performing a transcription reaction over each of the at least two DNA vector pools respectively:
performing a fragmentation reaction to the RNA molecules corresponding to the each of the at least two DNA vector pools.

26. The method of claim 21, wherein the performing the transcription reactions over the plurality of DNA vectors comprises:
performing a transcription reaction over each of the plurality of DNA vectors to thereby obtain an RNA molecule corresponding thereto.

27. The method of claim 14, wherein the capturing each strand of the at least one target nucleic acid sequence from the biological sample comprises:
contacting both the first RNA probe set and the second RNA probe set in the each of the at least one pair of RNA probe sets with the at least one target nucleic acid sequence in the biological sample; and
immobilizing the at least one target nucleic acid sequence on the solid support.

28. The method of claim 27, wherein each RNA probe in any of the first RNA probe set and the second RNA probe set in the each of the at least one pair of RNA probe sets has a length of about 100-150 nt, wherein:
the contacting both the first RNA probe set and the second RNA probe set in the each of the at least one pair of RNA probe sets with the at least one target nucleic acid sequence in the biological sample is performed at a temperature of about 62-70° C. and for about 6-24 hours.

29. The method of claim 28, wherein the contacting both the first RNA probe set and the second RNA probe set in the each of the at least one pair of RNA probe sets with the at least one target nucleic acid sequence in the biological sample is performed at a temperature of about 67.5° C.

30. The method of claim 27, wherein the first RNA probe set and the second RNA probe set in the each of the at least one pair of RNA probe sets respectively target a different portion of the duplex segment in the each of the at least one target nucleic acid sequence, and the contacting both the first RNA probe set and the second RNA probe set in the each of the at least one pair of RNA probe sets with the at least one target nucleic acid sequence in the biological sample comprises:
contacting both of the first RNA probe set and the second RNA probe set in each of the at least one pair of RNA probe sets with the at least one target nucleic acid sequence in a single hybridization reaction.

31. The method of claim 27, wherein the first RNA probe set and the second RNA probe set in the each of the at least one pair of RNA probe sets respectively target a substantially same portion of the duplex segment in the each of the at least one target nucleic acid sequence, and the contacting both the first RNA probe set and the second RNA probe set in the each of the at least one pair of RNA probe sets with the at least one target nucleic acid sequence in the biological sample comprises at least one round of:
contacting one of the first RNA probe set and the second RNA probe set in each of the at least one pair of RNA probe sets with the at least one target nucleic acid sequence in a first hybridization reaction; and
contacting another of the first RNA probe set and the second RNA probe set in each of the at least one pair of RNA probe sets with the at least one target nucleic acid sequence in a second hybridization reaction.

32. The method of claim 27, wherein the first RNA probe set and the second RNA probe set in the each of the at least one pair of RNA probe sets respectively target a substantially same portion of the duplex segment in the each of the at least one target nucleic acid sequence, and the contacting both the first RNA probe set and the second RNA probe set in the each of the at least one pair of RNA probe sets with the at least one target nucleic acid sequence in the biological sample comprises at least one round of:
separately contacting the first RNA probe set and the second RNA probe set in each of the at least one pair of RNA probe sets with the at least one target nucleic acid sequence in a third hybridization reaction and a fourth hybridization reaction, respectively; and
combining the third hybridization reaction and the fourth hybridization reaction to thereby allow a fifth hybridization reaction to proceed.

33. The method of claim 27, wherein one or more of the at least one target nucleic acid sequence in the biological sample are each in a polynucleotide containing at least one un-targeted sequence, wherein the method further comprises, prior to the contacting both the first RNA probe set and the second RNA probe set in the each of the at least one pair of RNA probe sets with the at least one target nucleic acid sequence in the biological sample:
contacting at least one blocking oligo with the at least one target nucleic acid sequence such that the at least one blocking oligo respectively hybridizes with, and thereby blocks, at least one strand of each of the at least one un-targeted sequence in the polynucleotide.

34. The method of claim 33, wherein the at least one un-targeted sequence in the polynucleotide comprises a first adaptor sequence and a second adaptor sequence flanking each of the one or more of the at least one target nucleic acid sequence, wherein:
the at least one blocking oligo is configured to respectively block one strand of the first adaptor sequence and one strand of the second adaptor sequence in the polynucleotide.

35. The method of claim 14, wherein the capturing each strand of the at least one target nucleic acid sequence from the biological sample comprises:
conjugating the at least one pair of RNA probe sets on the solid support via the immobilization portion labelled onto each RNA probe in any of the first RNA probe set and the second RNA probe set in the each of the at least one pair of RNA probe sets to thereby obtain at least one pair of solid support-conjugated RNA probe sets, each pair comprising a solid support-conjugated first RNA probe set and a solid support-conjugated second RNA probe set; and
contacting both the solid support-conjugated first RNA probe set and the solid support-conjugated second RNA probe set in the each of the at least one pair of solid support-conjugated RNA probe sets with the at least one target nucleic acid sequence.

36. The method of claim 35, wherein the contacting both the solid support-conjugated first RNA probe set and the solid support-conjugated second RNA probe set in the each of the at least one pair of solid support-conjugated RNA probe sets with the at least one target nucleic acid sequence comprises:
contacting one of the solid support-conjugated first RNA probe set and the solid support-conjugated second RNA probe set in the each of the at least one pair of solid support-conjugated RNA probe sets with the at least one target nucleic acid sequence in a sixth hybridization reaction; and
contacting another of the solid support-conjugated first RNA probe set and the solid support-conjugated second RNA probe set in the each of the at least one pair of solid support-conjugated RNA probe sets with the at least one target nucleic acid sequence in a seventh hybridization reaction.

37. The method of claim 35, wherein the contacting both the solid support-conjugated first RNA probe set and the solid support-conjugated second RNA probe set in the each of the at least one pair of solid support-conjugated RNA probe sets with the at least one target nucleic acid sequence comprises:
separately contacting the solid support-conjugated first RNA probe set and the solid support-conjugated second RNA probe set in the each of the at least one pair of solid support-conjugated RNA probe sets with the at least one target nucleic acid sequence in an eighth hybridization reaction and a ninth hybridization reaction, respectively; and
combining the eighth hybridization reaction and the ninth hybridization reaction to thereby allow a tenth hybridization reaction to proceed.

38. The method of claim 35, wherein the contacting both the solid support-conjugated first RNA probe set and the solid support-conjugated second RNA probe set in the each of the at least one pair of solid support-conjugated RNA probe sets with the at least one target nucleic acid sequence comprises:
contacting both of the solid support-conjugated first RNA probe set and the solid support-conjugated second RNA probe set in the each of the at least one pair of solid support-conjugated RNA probe sets with the at least one target nucleic acid sequence in a single hybridization reaction.


SEQUENCE LISTING (914 sequences)

SEQ ID NO: 1 - DNA, 23 bp, Homo Sapiens
gctcatttttgttaatggtggct

SEQ ID NO: 2 - DNA, 27 bp, Homo Sapiens
gaacaatgaataaaccattttgtctca

SEQ ID NO: 3 - DNA, 20 bp, Homo Sapiens
atcatgctgacgcttctcct

SEQ ID NO: 4 - DNA, 27 bp, Homo Sapiens
tgcagatttttaaaaagtctcttttcc

SEQ ID NO: 5 - DNA, 27 bp, Homo Sapiens
agttaataaggacatggtttctgttct

SEQ ID NO: 6 - DNA, 26 bp, Homo Sapiens
aggaaactagagttcattttcctttt

SEQ ID NO: 7 - DNA, 21 bp, Homo Sapiens
tcgcaagtctgaatcacaact

SEQ ID NO: 8 - DNA, 27 bp, Homo Sapiens
tgtgcattgagagtttttatactagtg

SEQ ID NO: 9 - DNA, 21 bp, Homo Sapiens
ccaggttagctggtaagagga

SEQ ID NO: 10 - DNA, 27 bp, Homo Sapiens
aagcttttcaggataatttctgttttt

SEQ ID NO: 11 - DNA, 22 bp, Homo Sapiens
aggatgatactggttgacacaa

SEQ ID NO: 12 - DNA, 25 bp, Homo Sapiens
tcagcttttccttaaagtcacttca

SEQ ID NO: 13 - DNA, 27 bp, Homo Sapiens
caccatatattaacttctgacatttgc

SEQ ID NO: 14 - DNA, 25 bp, Homo Sapiens
gctacaaaagagctcaacaattttt

SEQ ID NO: 15 - DNA, 25 bp, Homo Sapiens
gtgattttctaaaatagcaggctct

SEQ ID NO: 16 - DNA, 22 bp, Homo Sapiens
tcaccactgagcaaatgtttga

SEQ ID NO: 17 - DNA, 25 bp, Homo Sapiens
agcagttgtgattctttctttctac

SEQ ID NO: 18 - DNA, 27 bp, Homo Sapiens
ttggaagtataatagcagttcttttct

SEQ ID NO: 19 - DNA, 25 bp, Homo Sapiens
tgacagctatgctaatagtgtttct

SEQ ID NO: 20 - DNA, 20 bp, Homo Sapiens
tgggtgcttgggtgtgtata

SEQ ID NO: 21 - DNA, 22 bp, Homo Sapiens
tctgtcttctcctctcttcctt

SEQ ID NO: 22 - DNA, 23 bp, Homo Sapiens
tggttaggagaatgagttgttgt

SEQ ID NO: 23 - DNA, 22 bp, Homo Sapiens
tgcatctgtggcattaaatggt

SEQ ID NO: 24 - DNA, 24 bp, Homo Sapiens
atggtttttatgacacagagttgt

SEQ ID NO: 25 - DNA, 27 bp, Homo Sapiens
aggatcactttatatggatcacttttt

SEQ ID NO: 26 - DNA, 20 bp, Homo Sapiens
tgtgtacatggtgccagaat

SEQ ID NO: 27 - DNA, 26 bp, Homo Sapiens
agtcaatttttctttcttctcttgca

SEQ ID NO: 28 - DNA, 19 bp, Homo Sapiens
ttggaggccactgctatgg

SEQ ID NO: 29 - DNA, 27 bp, Homo Sapiens
aaatgtattcactgtcttctctttctt

SEQ ID NO: 30 - DNA, 23 bp, Homo Sapiens
tgtgtgactctaaattgactgca

SEQ ID NO: 31 - DNA, 27 bp, Homo Sapiens
tgtgtgacatgttctaatatagtcaca

SEQ ID NO: 32 - DNA, 22 bp, Homo Sapiens
atgcaaagtacctaactccact

SEQ ID NO: 33 - DNA, 20 bp, Homo Sapiens
tgtttgtgcctcactttgca

SEQ ID NO: 34 - DNA, 21 bp, Homo Sapiens
acacgtcttggggatatcact

SEQ ID NO: 35 - DNA, 26 bp, Homo Sapiens
actggtatggaaatattacagtcctg

SEQ ID NO: 36 - DNA, 25 bp, Homo Sapiens
gccatgttttacatttgagttcaca

SEQ ID NO: 37 - DNA, 20 bp, Homo Sapiens
acaaggctgagacctgagtt

SEQ ID NO: 38 - DNA, 22 bp, Homo Sapiens
tgtcactttgttctgtttgcag

SEQ ID NO: 39 - DNA, 20 bp, Homo Sapiens
tggctgttgtcactattgca

SEQ ID NO: 40 - DNA, 22 bp, Homo Sapiens
gctgtttctcttttctccacca

SEQ ID NO: 41 - DNA, 22 bp, Homo Sapiens
actgtgtattcatgttcccctt

SEQ ID NO: 42 - DNA, 25 bp, Homo Sapiens
ggatttaactttgtttttctctgcc

SEQ ID NO: 43 - DNA, 25 bp, Homo Sapiens
agacttcgagtgtttaaatgaaaga

SEQ ID NO: 44 - DNA, 26 bp, Homo Sapiens
tcagaagttctggaaatacttcattt

SEQ ID NO: 45 - DNA, 25 bp, Homo Sapiens
atgcttcagatgttgtatgtaatgt

SEQ ID NO: 46 - DNA, 21 bp, Homo Sapiens
ctactcatgcccctcaaactt

SEQ ID NO: 47 - DNA, 24 bp, Homo Sapiens
tcttaaatgatcctcttctcccag

SEQ ID NO: 48 - DNA, 25 bp, Homo Sapiens
gcgatgtgcatatataatggaaagg

SEQ ID NO: 49 - DNA, 20 bp, Homo Sapiens
aatcctgtttgcagcgtgag

SEQ ID NO: 50 - DNA, 26 bp, Homo Sapiens
actccattcagaaagtaattttcacc

SEQ ID NO: 51 - DNA, 24 bp, Homo Sapiens
tcaggatttttcctttctctctcc

SEQ ID NO: 52 - DNA, 21 bp, Homo Sapiens
gagttctaggctactgtggct

SEQ ID NO: 53 - DNA, 23 bp, Homo Sapiens
gtgaatacactttctgctggttt

SEQ ID NO: 54 - DNA, 22 bp, Homo Sapiens
aaccaagtcagtaaggccatat

SEQ ID NO: 55 - DNA, 23 bp, Homo Sapiens
aaacccttctgtttctttcacag

SEQ ID NO: 56 - DNA, 22 bp, Homo Sapiens
tcctgtgagtgttctgtatgtt

SEQ ID NO: 57 - DNA, 20 bp, Homo Sapiens
gctgggaacatttgtcattt

SEQ ID NO: 58 - DNA, 24 bp, Homo Sapiens
tgccgtttcgagaattttattcac

SEQ ID NO: 59 - DNA, 21 bp, Homo Sapiens
tgcacactgaaattaggacgt

SEQ ID NO: 60 - DNA, 23 bp, Homo Sapiens
tttttcccccttgatccttctag

SEQ ID NO: 61 - DNA, 20 bp, Homo Sapiens
acttattgagcatgcctggt

SEQ ID NO: 62 - DNA, 20 bp, Homo Sapiens
acgcagtgctaaccaagttc

SEQ ID NO: 63 - DNA, 25 bp, Homo Sapiens
ttttatatgatctttcctggttggc

SEQ ID NO: 64 - DNA, 20 bp, Homo Sapiens
aagaatgcctgggactgagg

SEQ ID NO: 65 - DNA, 27 bp, Homo Sapiens
aaagtgtagactaatgatgtgactttt

SEQ ID NO: 66 - DNA, 24 bp, Homo Sapiens
tgtactctttttcctgttgctgtt

SEQ ID NO: 67 - DNA, 24 bp, Homo Sapiens
aagcaaacttgttctgtttttacc

SEQ ID NO: 68 - DNA, 21 bp, Homo Sapiens
tgtgtgtttctgtgggtttct

SEQ ID NO: 69 - DNA, 22 bp, Homo Sapiens
tgtttttgtccctctctcttgc

SEQ ID NO: 70 - DNA, 26 bp, Homo Sapiens
acatactttgtatcatcttattggca

SEQ ID NO: 71 - DNA, 23 bp, Homo Sapiens
tgttttcctttgtgtcattccct

SEQ ID NO: 72 - DNA, 23 bp, Homo Sapiens
ggattcagtgaggaatgctttca

SEQ ID NO: 73 - DNA, 23 bp, Homo Sapiens
acacagaatttgttaagcaacgt

SEQ ID NO: 74 - DNA, 27 bp, Homo Sapiens
tctcatcataccactattattttgcag

SEQ ID NO: 75 - DNA, 20 bp, Homo Sapiens
acacagccttctcaacctct

SEQ ID NO: 76 - DNA, 25 bp, Homo Sapiens
ctctcggtgtatttctctacttacc

SEQ ID NO: 77 - DNA, 23 bp, Homo Sapiens
tcactgtgatagccttatctgct

SEQ ID NO: 78 - DNA, 24 bp, Homo Sapiens
gttaatcttaacctgtgctttgcc

SEQ ID NO: 79 - DNA, 20 bp, Homo Sapiens
actccaacactgttgctcct

SEQ ID NO: 80 - DNA, 20 bp, Homo Sapiens
gccagagtcctttagcccta

SEQ ID NO: 81 - DNA, 23 bp, Homo Sapiens
tcctgggtttcttttcaacttgt

SEQ ID NO: 82 - DNA, 24 bp, Homo Sapiens
tcctgaagtgagatcttacatcct

SEQ ID NO: 83 - DNA, 20 bp, Homo Sapiens
gaccagaaaggctcagttcc

SEQ ID NO: 84 - DNA, 23 bp, Homo Sapiens
aaacattgatttggcttttccca

SEQ ID NO: 85 - DNA, 20 bp, Homo Sapiens
tgtctccatttcccaccaca

SEQ ID NO: 86 - DNA, 26 bp, Homo Sapiens
atttactctggattttgctttttcag

SEQ ID NO: 87 - DNA, 22 bp, Homo Sapiens
tgagatgtttttgccttcacag

SEQ ID NO: 88 - DNA, 21 bp, Homo Sapiens
tcccccttgtcttaaaccaca

SEQ ID NO: 89 - DNA, 20 bp, Homo Sapiens
ttgacgtgccattctcttgc

SEQ ID NO: 90 - DNA, 20 bp, Homo Sapiens
tttgggacgacttattgccc

SEQ ID NO: 91 - DNA, 27 bp, Homo Sapiens
agagtaacttcaatgtctttattccat

SEQ ID NO: 92 - DNA, 20 bp, Homo Sapiens
gatctccggttgggattcct

SEQ ID NO: 93 - DNA, 23 bp, Homo Sapiens
gcattttctctgttcaacaagca

SEQ ID NO: 94 - DNA, 22 bp, Homo Sapiens
gtttgtgacataagcagaacgc

SEQ ID NO: 95 - DNA, 21 bp, Homo Sapiens
tgacagccagattcgtctttg

SEQ ID NO: 96 - DNA, 22 bp, Homo Sapiens
tggaagtctgtgagttctctga

SEQ ID NO: 97 - DNA, 26 bp, Homo Sapiens
atgttcttcctttaggtatagtctca

SEQ ID NO: 98 - DNA, 20 bp, Homo Sapiens
cctgaagcagtccaggactt

SEQ ID NO: 99 - DNA, 22 bp, Homo Sapiens
agcttaagaaaagtgacgtggt

SEQ ID NO: 100 - DNA, 27 bp, Homo Sapiens
tccttgagtgtaaggcaattaataact

SEQ ID NO: 101 - DNA, 24 bp, Homo Sapiens
gtctgtggattgacttcttttcct

SEQ ID NO: 102 - DNA, 27 bp, Homo Sapiens
tgagcctcatttattttctttttctcc

SEQ ID NO: 103 - DNA, 24 bp, Homo Sapiens
tcccttatgttcttcttctctcca

SEQ ID NO: 104 - DNA, 25 bp, Homo Sapiens
gcactttaatactaacaggaacagc

SEQ ID NO: 105 - DNA, 24 bp, Homo Sapiens
tgtcctcagtctgtactaaactca

SEQ ID NO: 106 - DNA, 22 bp, Homo Sapiens
agcagatttgtatgaaagccct

SEQ ID NO: 107 - DNA, 20 bp, Homo Sapiens
accctgttacacgcttgtaa

SEQ ID NO: 108 - DNA, 23 bp, Homo Sapiens
actgaatttgttttagggcacat

SEQ ID NO: 109 - DNA, 20 bp, Homo Sapiens
caccggtgtggctctttaac

SEQ ID NO: 110 - DNA, 20 bp, Homo Sapiens
ccgttgggttttctcttcca

SEQ ID NO: 111 - DNA, 23 bp, Homo Sapiens
ttctattcttccttgctttgtgc

SEQ ID NO: 112 - DNA, 21 bp, Homo Sapiens
aggatggtcttgtgtctgtgt

SEQ ID NO: 113 - DNA, 24 bp, Homo Sapiens
agttattttcaaacggtctggttt

SEQ ID NO: 114 - DNA, 20 bp, Homo Sapiens
aaatggcccttgtcttgcag

SEQ ID NO: 115 - DNA, 21 bp, Homo Sapiens
tctcccttcctcctttgaaca

SEQ ID NO: 116 - DNA, 23 bp, Homo Sapiens
agttaaagttcggttgttttcgt

SEQ ID NO: 117 - DNA, 20 bp, Homo Sapiens
gcaaggagccaggcattttt

SEQ ID NO: 118 - DNA, 21 bp, Homo Sapiens
tccattggattccttcgcaag

SEQ ID NO: 119 - DNA, 20 bp, Homo Sapiens
ttgtcagctttttgggggtc

SEQ ID NO: 120 - DNA, 23 bp, Homo Sapiens
ggtttacgtgcatattttctggc

SEQ ID NO: 121 - DNA, 20 bp, Homo Sapiens
ggcactttccttctggttca

SEQ ID NO: 122 - DNA, 24 bp, Homo Sapiens
tggtggtgcatactttatttgtct

SEQ ID NO: 123 - DNA, 23 bp, Homo Sapiens
aagtggtttcctcagatagagca

SEQ ID NO: 124 - DNA, 20 bp, Homo Sapiens
agcgtttcagctccagactc

SEQ ID NO: 125 - DNA, 20 bp, Homo Sapiens
agcaaccatttttgtgccca

SEQ ID NO: 126 - DNA, 20 bp, Homo Sapiens
tatgtctcctcttccctgcc

SEQ ID NO: 127 - DNA, 20 bp, Homo Sapiens
gtcctgctcctgatcctgtt

SEQ ID NO: 128 - DNA, 20 bp, Homo Sapiens
cttctccctggctctgactc

SEQ ID NO: 129 - DNA, 20 bp, Homo Sapiens
atctcaatcgcctgctctcc

SEQ ID NO: 130 - DNA, 22 bp, Homo Sapiens
agaccttttcctccctcattca

SEQ ID NO: 131 - DNA, 23 bp, Homo Sapiens
acatctgggattttgcttcattg

SEQ ID NO: 132 - DNA, 24 bp, Homo Sapiens
tcagaccacggtttctatttttca

SEQ ID NO: 133 - DNA, 25 bp, Homo Sapiens
atcttctgttacattctgcttcaca

SEQ ID NO: 134 - DNA, 20 bp, Homo Sapiens
ttttcaccccgctcccctta

SEQ ID NO: 135 - DNA, 20 bp, Homo Sapiens
tccttaaggcctctgtgctt

SEQ ID NO: 136 - DNA, 22 bp, Homo Sapiens
ggtcatttgctgtgtttgttga

SEQ ID NO: 137 - DNA, 27 bp, Homo Sapiens
ccataatataaagttgttgcgttttgt

SEQ ID NO: 138 - DNA, 22 bp, Homo Sapiens
gttctgagttagctgcacattt

SEQ ID NO: 139 - DNA, 20 bp, Homo Sapiens
ttctggccttgttaatggcg

SEQ ID NO: 140 - DNA, 23 bp, Homo Sapiens
aaatcccttcctctctttctcag

SEQ ID NO: 141 - DNA, 20 bp, Homo Sapiens
tacacccctgtcctctctgt

SEQ ID NO: 142 - DNA, 20 bp, Homo Sapiens
agacctcagacaaggcatct

SEQ ID NO: 143 - DNA, 20 bp, Homo Sapiens
actttctctcctgccctcac

SEQ ID NO: 144 - DNA, 24 bp, Homo Sapiens
cacttcagttatgtacctgatggg

SEQ ID NO: 145 - DNA, 20 bp, Homo Sapiens
ttccccattccccattccaa

SEQ ID NO: 146 - DNA, 20 bp, Homo Sapiens
tgctgaacgcatttggctta

SEQ ID NO: 147 - DNA, 21 bp, Homo Sapiens
tcattgtgtcttccctcctct

SEQ ID NO: 148 - DNA, 23 bp, Homo Sapiens
tgttgacaatgttttctcccaca

SEQ ID NO: 149 - DNA, 27 bp, Homo Sapiens
acatttttaatgctcctttctttgaca

SEQ ID NO: 150 - DNA, 21 bp, Homo Sapiens
agcaaaaggagtgacattcct

SEQ ID NO: 151 - DNA, 25 bp, Homo Sapiens
tctcatcatttcactgagatatgca

SEQ ID NO: 152 - DNA, 23 bp, Homo Sapiens
tgaacttgtcacttcattggtca

SEQ ID NO: 153 - DNA, 23 bp, Homo Sapiens
aggcagcctttataaaagcaaat

SEQ ID NO: 154 - DNA, 21 bp, Homo Sapiens
acccattttccttcctggaca

SEQ ID NO: 155 - DNA, 21 bp, Homo Sapiens
aaaaatttcccctgcgcttag

SEQ ID NO: 156 - DNA, 20 bp, Homo Sapiens
agacatgatgcttcgcttga

SEQ ID NO: 157 - DNA, 20 bp, Homo Sapiens
tgcacgttgtttgtagctgt

SEQ ID NO: 158 - DNA, 22 bp, Homo Sapiens
gtttcccctggatttatgtggt

SEQ ID NO: 159 - DNA, 20 bp, Homo Sapiens
acgtgcatgtcctttttccc

SEQ ID NO: 160 - DNA, 25 bp, Homo Sapiens
tgtctctacctcctacatcttatct

SEQ ID NO: 161 - DNA, 25 bp, Homo Sapiens
ggttgtttctatttgctaatgctgt

SEQ ID NO: 162 - DNA, 25 bp, Homo Sapiens
agcacttcctgaaataatttcacct

SEQ ID NO: 163 - DNA, 20 bp, Homo Sapiens
atgggcctcactgtctgttt

SEQ ID NO: 164 - DNA, 20 bp, Homo Sapiens
gtcctcatggctctgtgact

SEQ ID NO: 165 - DNA, 20 bp, Homo Sapiens
cccacctgcattgttcatca

SEQ ID NO: 166 - DNA, 27 bp, Homo Sapiens
ggataactatgttcttccttttcatca

SEQ ID NO: 167 - DNA, 20 bp, Homo Sapiens
ctggcacacttcttcacctc

SEQ ID NO: 168 - DNA, 20 bp, Homo Sapiens
cctcagggatggtagtgaca

SEQ ID NO: 169 - DNA, 25 bp, Homo Sapiens
catccatggaatatgttcttttgca

SEQ ID NO: 170 - DNA, 20 bp, Homo Sapiens
ccttgcactcttgtggttgt

SEQ ID NO: 171 - DNA, 23 bp, Homo Sapiens
aaggtttccaattcacctttcag

SEQ ID NO: 172 - DNA, 20 bp, Homo Sapiens
ggtccccatccattcttcct

SEQ ID NO: 173 - DNA, 22 bp, Homo Sapiens
ccaatctgcttatgaccaggag

SEQ ID NO: 174 - DNA, 21 bp, Homo Sapiens
ggtgggattttgttgtttgca

SEQ ID NO: 175 - DNA, 20 bp, Homo Sapiens
tgaccaatttggcttcgtcc

SEQ ID NO: 176 - DNA, 21 bp, Homo Sapiens
tcaaagctgcttctgtcatct

SEQ ID NO: 177 - DNA, 20 bp, Homo Sapiens
ggctaatggttctcagagct

SEQ ID NO: 178 - DNA, 22 bp, Homo Sapiens
gcctttcaattcactgtcctca

SEQ ID NO: 179 - DNA, 20 bp, Homo Sapiens
tcagcagggtttttcttgct

SEQ ID NO: 180 - DNA, 20 bp, Homo Sapiens
ggttttcctctccttcccca

SEQ ID NO: 181 - DNA, 20 bp, Homo Sapiens
ccatgacactccttccacct

SEQ ID NO: 182 - DNA, 23 bp, Homo Sapiens
tgtaattcctggcttctaggttt

SEQ ID NO: 183 - DNA, 20 bp, Homo Sapiens
cgtgttcccgtttcctcttg

SEQ ID NO: 184 - DNA, 21 bp, Homo Sapiens
cctctgtgtatctccttccca

SEQ ID NO: 185 - DNA, 20 bp, Homo Sapiens
gctttctttttgctccccca

SEQ ID NO: 186 - DNA, 21 bp, Homo Sapiens
acttttaccctggatttgccc

SEQ ID NO: 187 - DNA, 21 bp, Homo Sapiens
ggtggggttttgttaacgtga

SEQ ID NO: 188 - DNA, 24 bp, Homo Sapiens
agtgtaaagttaaccttgctgtgt

SEQ ID NO: 189 - DNA, 20 bp, Homo Sapiens
gtatcaaggctgccctgact

SEQ ID NO: 190 - DNA, 20 bp, Homo Sapiens
ttcaggccaccaacctcatt

SEQ ID NO: 191 - DNA, 20 bp, Homo Sapiens
gtgctgattccctgatgtgc

SEQ ID NO: 192 - DNA, 20 bp, Homo Sapiens
accaacatggatggagtggt

SEQ ID NO: 193 - DNA, 19 bp, Homo Sapiens
tgcccaccctaatcctgtg

SEQ ID NO: 194 - DNA, 20 bp, Homo Sapiens
ctgcctctcttttctcccca

SEQ ID NO: 195 - DNA, 20 bp, Homo Sapiens
gaagtcatgggctgcttgtc

SEQ ID NO: 196 - DNA, 20 bp, Homo Sapiens
ccaagctgtgaaggcctttt

SEQ ID NO: 197 - DNA, 20 bp, Homo Sapiens
cctccttctcacgtgtctgt

SEQ ID NO: 198 - DNA, 20 bp, Homo Sapiens
agcagagtgacccagtgatg

SEQ ID NO: 199 - DNA, 20 bp, Homo Sapiens
tcctctctggatcctcgtga

SEQ ID NO: 200 - DNA, 20 bp, Homo Sapiens
ggagctgctcctcatcctac

SEQ ID NO: 201 - DNA, 23 bp, Homo Sapiens
tgaagtttttgtctgtttctccc

SEQ ID NO: 202 - DNA, 20 bp, Homo Sapiens
actggatctgcttcacacct

SEQ ID NO: 203 - DNA, 20 bp, Homo Sapiens
ctttccctcattccctcccc

SEQ ID NO: 204 - DNA, 20 bp, Homo Sapiens
ttgggcctgtgttatctcct

SEQ ID NO: 205 - DNA, 20 bp, Homo Sapiens
cctgacccttctccctatcc

SEQ ID NO: 206 - DNA, 20 bp, Homo Sapiens
tccctttctcccctctttga

SEQ ID NO: 207 - DNA, 20 bp, Homo Sapiens
actcaagtccctttcccctc

SEQ ID NO: 208 - DNA, 20 bp, Homo Sapiens
tcttcacttcagttgcccct

SEQ ID NO: 209 - DNA, 20 bp, Homo Sapiens
ccacactgagcctttttccc

SEQ ID NO: 210 - DNA, 19 bp, Homo Sapiens
catgatgcgctgtgtgtcc

SEQ ID NO: 211 - DNA, 20 bp, Homo Sapiens
aggaagggcagtgaggattc

SEQ ID NO: 212 - DNA, 20 bp, Homo Sapiens
caaaaagtgccagccctcac

SEQ ID NO: 213 - DNA, 20 bp, Homo Sapiens
actaagttgccacaggacct

SEQ ID NO: 214 - DNA, 20 bp, Homo Sapiens
gggctctggggcattaacat

SEQ ID NO: 215 - DNA, 20 bp, Homo Sapiens
cgaagtctcgctcttttccc

SEQ ID NO: 216 - DNA, 25 bp, Homo Sapiens
tgttttgttgttcttggcattttct

SEQ ID NO: 217 - DNA, 20 bp, Homo Sapiens
acctgcccagatccttaacc

SEQ ID NO: 218 - DNA, 20 bp, Homo Sapiens
ctctctccacccaaaccctt

SEQ ID NO: 219 - DNA, 21 bp, Homo Sapiens
tcccctcctcttcttgttctc

SEQ ID NO: 220 - DNA, 21 bp, Homo Sapiens
cgacttcagtcttccacttcc

SEQ ID NO: 221 - DNA, 20 bp, Homo Sapiens
tcagcaggaagtgttgacct

SEQ ID NO: 222 - DNA, 20 bp, Homo Sapiens
gaggccaggcatttttcact

SEQ ID NO: 223 - DNA, 20 bp, Homo Sapiens
caacacagtctctccctcca

SEQ ID NO: 224 - DNA, 19 bp, Homo Sapiens
ttgctgctgcctcgcttat

SEQ ID NO: 225 - DNA, 20 bp, Homo Sapiens
actgttctgacacaccccac

SEQ ID NO: 226 - DNA, 20 bp, Homo Sapiens
ctctaaatccctcgccctgg

SEQ ID NO: 227 - DNA, 20 bp, Homo Sapiens
aacctctacccacccattcc

SEQ ID NO: 228 - DNA, 20 bp, Homo Sapiens
tcctctctccccactctcag

SEQ ID NO: 229 - DNA, 18 bp, Homo Sapiens
aaaaacgggtggttgggc

SEQ ID NO: 230 - DNA, 20 bp, Homo Sapiens
acagacagccgaacagacac

SEQ ID NO: 231 - DNA, 25 bp, Homo Sapiens
ttctttttagtctagtgctccacta

SEQ ID NO: 232 - DNA, 19 bp, Homo Sapiens
taagcccgggacttccttg

SEQ ID NO: 233 - DNA, 20 bp, Homo Sapiens
ggggttttgattggctgagg

SEQ ID NO: 234 - DNA, 20 bp, Homo Sapiens
cctagggtgaggcttatggg

SEQ ID NO: 235 - DNA, 20 bp, Homo Sapiens
gggtggccattaacacacaa

SEQ ID NO: 236 - DNA, 20 bp, Homo Sapiens
ctgcgaggaggggagaattc

SEQ ID NO: 237 - DNA, 20 bp, Homo Sapiens
tcccattcccgtgtttcctt

SEQ ID NO: 238 - DNA, 20 bp, Homo Sapiens
aggctctgatgtgcttctct

SEQ ID NO: 239 - DNA, 20 bp, Homo Sapiens
gttttctgtctgcctctgcc

SEQ ID NO: 240 - DNA, 20 bp, Homo Sapiens
accctcaccctaaatctggc

SEQ ID NO: 241 - DNA, 19 bp, Homo Sapiens
ctgagcctgccctactctg

SEQ ID NO: 242 - DNA, 18 bp, Homo Sapiens
cgcgcgtacacacacaca

SEQ ID NO: 243 - DNA, 20 bp, Homo Sapiens
cctctctccttctgcctcag

SEQ ID NO: 244 - DNA, 22 bp, Homo Sapiens
tcgctgttagacatctctctca

SEQ ID NO: 245 - DNA, 20 bp, Homo Sapiens
tgagaccccttcagacccta

SEQ ID NO: 246 - DNA, 20 bp, Homo Sapiens
gtgtttccttggggtcatgg

SEQ ID NO: 247 - DNA, 18 bp, Homo Sapiens
ctcgctcatccccgaggg

SEQ ID NO: 248 - DNA, 20 bp, Homo Sapiens
ggcagctccgggtctataaa

SEQ ID NO: 249 - DNA, 20 bp, Homo Sapiens
ccctcaccttcccctctttt

SEQ ID NO: 250 - DNA, 20 bp, Homo Sapiens
ctgtccttccctgacctcag

SEQ ID NO: 251 - DNA, 18 bp, Homo Sapiens
tggtctctcggcgggaag

SEQ ID NO: 252 - DNA, 20 bp, Homo Sapiens
tgagttaacggctgcctctt

SEQ ID NO: 253 - DNA, 20 bp, Homo Sapiens
cacctggctccactgtgtag

SEQ ID NO: 254 - DNA, 20 bp, Homo Sapiens
cccctgctaatgtctgaggt

SEQ ID NO: 255 - DNA, 18 bp, Homo Sapiens
ctggccacactgggtctc

SEQ ID NO: 256 - DNA, 20 bp, Homo Sapiens
cactgtggccttgtttcctg

SEQ ID NO: 257 - DNA, 18 bp, Homo Sapiens
ctcactcctcccctgctc

SEQ ID NO: 258 - DNA, 20 bp, Homo Sapiens
tagttggcgagtgggcttta

SEQ ID NO: 259 - DNA, 19 bp, Homo Sapiens
ctggtttagcgacacgagc

SEQ ID NO: 260 - DNA, 20 bp, Homo Sapiens
cctctgctgactctgtctcc

SEQ ID NO: 261 - DNA, 19 bp, Homo Sapiens
cattggttgcggccatctc

SEQ ID NO: 262 - DNA, 18 bp, Homo Sapiens
ccactgcaacccgactcc

SEQ ID NO: 263 - DNA, 18 bp, Homo Sapiens
caggagggcggggtaaag

SEQ ID NO: 264 - DNA, 18 bp, Homo Sapiens
cgcctcttcccaccctag

SEQ ID NO: 265 - DNA, 18 bp, Homo Sapiens
cgtgaccgacatgtggct

SEQ ID NO: 266 - DNA, 20 bp, Homo Sapiens
ctgagacctggggactgatc

SEQ ID NO: 267 - DNA, 20 bp, Homo Sapiens
agggtttccttctcgctgat

SEQ ID NO: 268 - DNA, 18 bp, Homo Sapiens
aggtgtggtgttgcccac

SEQ ID NO: 269 - DNA, 20 bp, Homo Sapiens
tacccactccatttcccacc

SEQ ID NO: 270 - DNA, 18 bp, Homo Sapiens
ctgggctggcgtatgacg

SEQ ID NO: 271 - DNA, 20 bp, Homo Sapiens
ccgctcagtgtctctctctt

SEQ ID NO: 272 - DNA, 19 bp, Homo Sapiens
caggaagcctgtgttccgt

SEQ ID NO: 273 - DNA, 20 bp, Homo Sapiens
gaacttgccggttaagcagg

SEQ ID NO: 274 - DNA, 20 bp, Homo Sapiens
gtatcaacgctctgtgggtc

SEQ ID NO: 275 - DNA, 20 bp, Homo Sapiens
tgcttctcttccttctcccc

SEQ ID NO: 276 - DNA, 18 bp, Homo Sapiens
ctttgtgtgccccgctcc

SEQ ID NO: 277 - DNA, 19 bp, Homo Sapiens
aggcctcttgtttcctccc

SEQ ID NO: 278 - DNA, 20 bp, Homo Sapiens
ctatgggtgcccttctccac

SEQ ID NO: 279 - DNA, 19 bp, Homo Sapiens
gaggacctgtgggactctg

SEQ ID NO: 280 - DNA, 20 bp, Homo Sapiens
gggtgagactgacctctctt

SEQ ID NO: 281 - DNA, 18 bp, Homo Sapiens
ctgcagttcgcttgtgcc

SEQ ID NO: 282 - DNA, 18 bp, Homo Sapiens
cacccaccgctgtgttgc

SEQ ID NO: 283 - DNA, 19 bp, Homo Sapiens
aggaagggagcctcaaagg

SEQ ID NO: 284 - DNA, 19 bp, Homo Sapiens
gactctcctgtctccgctc

SEQ ID NO: 285 - DNA, 18 bp, Homo Sapiens
cctcctctgcgttcgacg

SEQ ID NO: 286 - DNA, 20 bp, Homo Sapiens
actacatttcccaggaggca

SEQ ID NO: 287 - DNA, 18 bp, Homo Sapiens
ccgtccgcgctacatact

SEQ ID NO: 288 - DNA, 20 bp, Homo Sapiens
cagtggctcaggaaaccaag

SEQ ID NO: 289 - DNA, 20 bp, Homo Sapiens
gctgatcctccaccttcctt

SEQ ID NO: 290 - DNA, 19 bp, Homo Sapiens
gaacatggtgcgcaggttc

SEQ ID NO: 291 - DNA, 20 bp, Homo Sapiens
gtgtgttgggggatagcctc

SEQ ID NO: 292 - DNA, 18 bp, Homo Sapiens
cacgtctgcccctctctc

SEQ ID NO: 293 - DNA, 19 bp, Homo Sapiens
ggcagaagagaggcagaca

SEQ ID NO: 294 - DNA, 20 bp, Homo Sapiens
tattaccggcagaaccagca

SEQ ID NO: 295 - DNA, 20 bp, Homo Sapiens
cacccggttccatctacctt

SEQ ID NO: 296 - DNA, 18 bp, Homo Sapiens
cgatgagggtctggccag

SEQ ID NO: 297 - DNA, 18 bp, Homo Sapiens
cgctgctgccttgatggg

SEQ ID NO: 298 - DNA, 20 bp, Homo Sapiens
actcactgaccctctccctt

SEQ ID NO: 299 - DNA, 20 bp, Homo Sapiens
agcgaggacatctggaagaa

SEQ ID NO: 300 - DNA, 19 bp, Homo Sapiens
caagaggccaatgaggggg

SEQ ID NO: 301 - DNA, 20 bp, Homo Sapiens
ctacctgtaactgggcctgt

SEQ ID NO: 302 - DNA, 18 bp, Homo Sapiens
gatggcgcctcagaagca

SEQ ID NO: 303 - DNA, 18 bp, Homo Sapiens
gggctccgtagacgcttt

SEQ ID NO: 304 - DNA, 20 bp, Homo Sapiens
ctgccttctcccctgaagag

SEQ ID NO: 305 - DNA, 27 bp, Homo Sapiens
agttgttttagaagatatttgcaagca

SEQ ID NO: 306 - DNA, 27 bp, Homo Sapiens
tcttatcttcactgaaaacataaacct

SEQ ID NO: 307 - DNA, 25 bp, Homo Sapiens
actgaaagctaggcacattttatga

SEQ ID NO: 308 - DNA, 23 bp, Homo Sapiens
gggggaaaagaacatctgaaaat

SEQ ID NO: 309 - DNA, 26 bp, Homo Sapiens
tgagaatacatttccaaacttgtcct

SEQ ID NO: 310 - DNA, 25 bp, Homo Sapiens
acattagcaattaagaacaccatct

SEQ ID NO: 311 - DNA, 23 bp, Homo Sapiens
tggtaaaacacaatccttcacga

SEQ ID NO: 312 - DNA, 24 bp, Homo Sapiens
acctgtagttcaactaaacagagg

SEQ ID NO: 313 - DNA, 20 bp, Homo Sapiens
acgcaactgacaggaggaat

SEQ ID NO: 314 - DNA, 23 bp, Homo Sapiens
tgccttatttccctattgatgca

SEQ ID NO: 315 - DNA, 25 bp, Homo Sapiens
tcactaaatacgtttcacaggtaga

SEQ ID NO: 316 - DNA, 25 bp, Homo Sapiens
tcagaaacgatcagttgaaatttca

SEQ ID NO: 317 - DNA, 27 bp, Homo Sapiens
tgcataagttatcaaaacacttaaggt

SEQ ID NO: 318 - DNA, 23 bp, Homo Sapiens
tttatgctctcccaataagctcc

SEQ ID NO: 319 - DNA, 19 bp, Homo Sapiens
gcgcccggctgaaattttt

SEQ ID NO: 320 - DNA, 24 bp, Homo Sapiens
ctcaaaggtacatgagaaaggtga

SEQ ID NO: 321 - DNA, 25 bp, Homo Sapiens
acaatagtacggtaatgaagaagct

SEQ ID NO: 322 - DNA, 24 bp, Homo Sapiens
atggctacatgtaactagtgagat

SEQ ID NO: 323 - DNA, 27 bp, Homo Sapiens
tcctagtcctttaaaacttttacgtga

SEQ ID NO: 324 - DNA, 22 bp, Homo Sapiens
actttcaaccactctgaaaggt

SEQ ID NO: 325 - DNA, 23 bp, Homo Sapiens
actttgcactgaaaaagtacaca

SEQ ID NO: 326 - DNA, 20 bp, Homo Sapiens
gacagctccttcaagaggga

SEQ ID NO: 327 - DNA, 21 bp, Homo Sapiens
catacaggttgccttactggt

SEQ ID NO: 328 - DNA, 26 bp, Homo Sapiens
agtttaaaatcatatgcacaacctca

SEQ ID NO: 329 - DNA, 25 bp, Homo Sapiens
gcgggtgatacttctttaatactca

SEQ ID NO: 330 - DNA, 20 bp, Homo Sapiens
tgcacccccataaatccaaa

SEQ ID NO: 331 - DNA, 22 bp, Homo Sapiens
acagacagaaattcactctgca

SEQ ID NO: 332 - DNA, 25 bp, Homo Sapiens
aacaaaccttatttcccacatgtaa

SEQ ID NO: 333 - DNA, 24 bp, Homo Sapiens
tcaataatgcatttccactccaaa

SEQ ID NO: 334 - DNA, 24 bp, Homo Sapiens
tgaagtgatcggaatacatgtaga

SEQ ID NO: 335 - DNA, 21 bp, Homo Sapiens
ggtcctgcaccagtaatatgc

SEQ ID NO: 336 - DNA, 20 bp, Homo Sapiens
gcctatgtgacagcaaacca

SEQ ID NO: 337 - DNA, 22 bp, Homo Sapiens
tcagtggcatcttttcacaagt

SEQ ID NO: 338 - DNA, 23 bp, Homo Sapiens
tcaggggtatctcacatactagc

SEQ ID NO: 339 - DNA, 21 bp, Homo Sapiens
ggaacaaagaaaaggccagga

SEQ ID NO: 340 - DNA, 25 bp, Homo Sapiens
tctatggtaaaagatctcaggtcat

SEQ ID NO: 341 - DNA, 25 bp, Homo Sapiens
tcaaggaaagctgatacctatttca

SEQ ID NO: 342 - DNA, 20 bp, Homo Sapiens
ttccaacctccaatgaccca

SEQ ID NO: 343 - DNA, 24 bp, Homo Sapiens
tgtccttgtgaaataaaaagaccc

SEQ ID NO: 344 - DNA, 24 bp, Homo Sapiens
aaacccactaatacttgaaggtca

SEQ ID NO: 345 - DNA, 20 bp, Homo Sapiens
cccacttccaaactcaagcc

SEQ ID NO: 346 - DNA, 24 bp, Homo Sapiens
aacaaaaacctgagaaaccagaac

SEQ ID NO: 347 - DNA, 20 bp, Homo Sapiens
taaattgcaaagcccacccc

SEQ ID NO: 348 - DNA, 21 bp, Homo Sapiens
tgaggggaacatatgtgcaac

SEQ ID NO: 349 - DNA, 23 bp, Homo Sapiens
acgtttacatactgaacacaggt

SEQ ID NO: 350 - DNA, 21 bp, Homo Sapiens
ccgagcagtcaaatgaactca

SEQ ID NO: 351 - DNA, 23 bp, Homo Sapiens
acagcaatcgtgaacaaatacct

SEQ ID NO: 352 - DNA, 27 bp, Homo Sapiens
ttttcattcgtatatgcttttcaaaca

SEQ ID NO: 353 - DNA, 22 bp, Homo Sapiens
tgaaacctgagaagaaggatgt

SEQ ID NO: 354 - DNA, 20 bp, Homo Sapiens
aggacttcactgtgacctgg

SEQ ID NO: 355 - DNA, 20 bp, Homo Sapiens
tgcttcaatggaggagctca

SEQ ID NO: 356 - DNA, 22 bp, Homo Sapiens
ggaccaagacatcacattccag

SEQ ID NO: 357 - DNA, 20 bp, Homo Sapiens
aatgtgggatcctcgcctta

SEQ ID NO: 358 - DNA, 27 bp, Homo Sapiens
cagtataattaacccaatattcggagt

SEQ ID NO: 359 - DNA, 20 bp, Homo Sapiens
tgtgttattggcaggaacgt

SEQ ID NO: 360 - DNA, 22 bp, Homo Sapiens
agaaagtacagaagaaggggga

SEQ ID NO: 361 - DNA, 23 bp, Homo Sapiens
tgtactgaaatgccaatggaact

SEQ ID NO: 362 - DNA, 21 bp, Homo Sapiens
tgtcccttcaacatcaaccaa

SEQ ID NO: 363 - DNA, 22 bp, Homo Sapiens
tgaaaccctcatgttaagcaac

SEQ ID NO: 364 - DNA, 18 bp, Homo Sapiens
aagccgtccgagatgacc

SEQ ID NO: 365 - DNA, 20 bp, Homo Sapiens
ggccaatctgctctaaacca

SEQ ID NO: 366 - DNA, 24 bp, Homo Sapiens
tgcaaaccacaaaagtatactcca

SEQ ID NO: 367 - DNA, 22 bp, Homo Sapiens
acaaagcacgatatgaagcaca

SEQ ID NO: 368 - DNA, 23 bp, Homo Sapiens
tcctaaacgtaagaagcaacact

SEQ ID NO: 369 - DNA, 23 bp, Homo Sapiens
tatgtaagacacgagacactgga

SEQ ID NO: 370 - DNA, 24 bp, Homo Sapiens
tcacaaccattaaaacaggagaca

SEQ ID NO: 371 - DNA, 25 bp, Homo Sapiens
agacaacgtcttcctatgatagaaa

SEQ ID NO: 372 - DNA, 23 bp, Homo Sapiens
cccaagatttaagaccaaaggct

SEQ ID NO: 373 - DNA, 27 bp, Homo Sapiens
tcaaaatagaatttagttgatggaggg

SEQ ID NO: 374 - DNA, 20 bp, Homo Sapiens
gcaatcccaggccagagata

SEQ ID NO: 375 - DNA, 27 bp, Homo Sapiens
tggagtttttctattaaccaggttatt

SEQ ID NO: 376 - DNA, 21 bp, Homo Sapiens
tgcagatatggcatcaacaga

SEQ ID NO: 377 - DNA, 20 bp, Homo Sapiens
ctccaaacctctacctggca

SEQ ID NO: 378 - DNA, 20 bp, Homo Sapiens
tgtgcagtccaggaaacaga

SEQ ID NO: 379 - DNA, 23 bp, Homo Sapiens
ttatgctgcttatcatacccagt

SEQ ID NO: 380 - DNA, 22 bp, Homo Sapiens
tcatgtccccagaaaatccagt

SEQ ID NO: 381 - DNA, 24 bp, Homo Sapiens
ctgactctagatacctggctaaac

SEQ ID NO: 382 - DNA, 27 bp, Homo Sapiens
aaaaaggcaccatctaaaagaaatagt

SEQ ID NO: 383 - DNA, 22 bp, Homo Sapiens
cttggagcaagtaagagcatgt

SEQ ID NO: 384 - DNA, 22 bp, Homo Sapiens
cgcagacaaatttcaggaagga

SEQ ID NO: 385 - DNA, 23 bp, Homo Sapiens
gcctacttcatccaaaatagcca

SEQ ID NO: 386 - DNA, 27 bp, Homo Sapiens
tctctaacacgactataattttcctct

SEQ ID NO: 387 - DNA, 24 bp, Homo Sapiens
tcaagagataaagtccataagcct

SEQ ID NO: 388 - DNA, 19 bp, Homo Sapiens
cccatttggaagcagctcg

SEQ ID NO: 389 - DNA, 20 bp, Homo Sapiens
tggctaaccaagtctcagga

SEQ ID NO: 390 - DNA, 25 bp, Homo Sapiens
ccagttatatcacaaataaagcccc

SEQ ID NO: 391 - DNA, 20 bp, Homo Sapiens
atctgaagatggggctggtg

SEQ ID NO: 392 - DNA, 24 bp, Homo Sapiens
aacattgcatattacccacaaaga

SEQ ID NO: 393 - DNA, 25 bp, Homo Sapiens
acaacaggaagtaaactcattttcc

SEQ ID NO: 394 - DNA, 20 bp, Homo Sapiens
agaattcaggcaatcaccca

SEQ ID NO: 395 - DNA, 21 bp, Homo Sapiens
agggtgtttctgtaacctcca

SEQ ID NO: 396 - DNA, 20 bp, Homo Sapiens
accaatttcctgtgcagaga

SEQ ID NO: 397 - DNA, 20 bp, Homo Sapiens
acagaaagctcccactcctc

SEQ ID NO: 398 - DNA, 25 bp, Homo Sapiens
agacaattcaagcttcagaatctct

SEQ ID NO: 399 - DNA, 24 bp, Homo Sapiens
ctcctctcagtcttctaaaatggt

SEQ ID NO: 400 - DNA, 21 bp, Homo Sapiens
tggaagggtttatatcgggct

SEQ ID NO: 401 - DNA, 23 bp, Homo Sapiens
tctgaaccacaatcaacttctcc

SEQ ID NO: 402 - DNA, 21 bp, Homo Sapiens
aacactgtgaaaagcaaagct

SEQ ID NO: 403 - DNA, 22 bp, Homo Sapiens
tctactgccaatgccttagttc

SEQ ID NO: 404 - DNA, 20 bp, Homo Sapiens
gtggcaaggttgaatcagca

SEQ ID NO: 405 - DNA, 25 bp, Homo Sapiens
tgctttaaaatctactcctaccagg

SEQ ID NO: 406 - DNA, 21 bp, Homo Sapiens
tggagccacataacacattca

SEQ ID NO: 407 - DNA, 22 bp, Homo Sapiens
agagaatgatgcaatttggggt

SEQ ID NO: 408 - DNA, 21 bp, Homo Sapiens
tttgcagccagaatctcttcc

SEQ ID NO: 409 - DNA, 24 bp, Homo Sapiens
agagaacagagaaagcttgaaact

SEQ ID NO: 410 - DNA, 20 bp, Homo Sapiens
cactgagagccttgaaagcc

SEQ ID NO: 411 - DNA, 22 bp, Homo Sapiens
ggaaacatcctgcacgttaagt

SEQ ID NO: 412 - DNA, 20 bp, Homo Sapiens
acactgacattgggagttgg

SEQ ID NO: 413 - DNA, 25 bp, Homo Sapiens
tgtacttaccacaacaaccttatct

SEQ ID NO: 414 - DNA, 20 bp, Homo Sapiens
gggccttgcagtaaaaggag

SEQ ID NO: 415 - DNA, 23 bp, Homo Sapiens
gcttgatctggagttaatcgaga

SEQ ID NO: 416 - DNA, 20 bp, Homo Sapiens
gtgggttttagcttgtcgca

SEQ ID NO: 417 - DNA, 20 bp, Homo Sapiens
ctggctccagaatccttcct

SEQ ID NO: 418 - DNA, 20 bp, Homo Sapiens
aagggggattctatgcctgg

SEQ ID NO: 419 - DNA, 21 bp, Homo Sapiens
agcatcctcccaaaagaagga

SEQ ID NO: 420 - DNA, 24 bp, Homo Sapiens
aatacagtaaggagtggagaagtc

SEQ ID NO: 421 - DNA, 23 bp, Homo Sapiens
cctgacaaatccagagtatacgc

SEQ ID NO: 422 - DNA, 24 bp, Homo Sapiens
agacccattaaaacctattcctca

SEQ ID NO: 423 - DNA, 21 bp, Homo Sapiens
ggcaaaagcctttgatgaagc

SEQ ID NO: 424 - DNA, 20 bp, Homo Sapiens
gtggaataacacctccagcc

SEQ ID NO: 425 - DNA, 25 bp, Homo Sapiens
tgatttttaaagtagcagaatggca

SEQ ID NO: 426 - DNA, 21 bp, Homo Sapiens
tgagtcacacaccaatgaaga

SEQ ID NO: 427 - DNA, 23 bp, Homo Sapiens
gtgaaatatgcaacagcttctca

SEQ ID NO: 428 - DNA, 23 bp, Homo Sapiens
tttacacggaatttcagtggtgg

SEQ ID NO: 429 - DNA, 21 bp, Homo Sapiens
acgaatcacttcttcagggga

SEQ ID NO: 430 - DNA, 20 bp, Homo Sapiens
aaggaaagccccttgagttt

SEQ ID NO: 431 - DNA, 25 bp, Homo Sapiens
acaacccacagtattttaaattgca

SEQ ID NO: 432 - DNA, 21 bp, Homo Sapiens
ccccagtaagtcccttcaaat

SEQ ID NO: 433 - DNA, 24 bp, Homo Sapiens
accacatatctgctatgtcttcct

SEQ ID NO: 434 - DNA, 24 bp, Homo Sapiens
tgactgaatgagaacttaagtggg

SEQ ID NO: 435 - DNA, 21 bp, Homo Sapiens
ccctcccatcttcctctaacc

SEQ ID NO: 436 - DNA, 20 bp, Homo Sapiens
acactggcatgcaacatgtt

SEQ ID NO: 437 - DNA, 20 bp, Homo Sapiens
aaacaggcctgagaaagctt

SEQ ID NO: 438 - DNA, 19 bp, Homo Sapiens
atccgtgatctgcaggcat

SEQ ID NO: 439 - DNA, 19 bp, Homo Sapiens
ccacacctggccaagctta

SEQ ID NO: 440 - DNA, 23 bp, Homo Sapiens
tctgtcatcacactcaaagatgc

SEQ ID NO: 441 - DNA, 18 bp, Homo Sapiens
tctgacacgcaagcccag

SEQ ID NO: 442 - DNA, 19 bp, Homo Sapiens
gcgcagaaagtgacagtgc

SEQ ID NO: 443 - DNA, 21 bp, Homo Sapiens
ccccctcgattgttaacatga

SEQ ID NO: 444 - DNA, 20 bp, Homo Sapiens
tcatgaacttcccacacagc

SEQ ID NO: 445 - DNA, 20 bp, Homo Sapiens
tcccaacaccggaaaggata

SEQ ID NO: 446 - DNA, 20 bp, Homo Sapiens
catcccagccatacaggact

SEQ ID NO: 447 - DNA, 20 bp, Homo Sapiens
ctatgctgcagctgttaccc

SEQ ID NO: 448 - DNA, 20 bp, Homo Sapiens
tggaaggcagtaaaggctga

SEQ ID NO: 449 - DNA, 20 bp, Homo Sapiens
acactctgacaggtcaagca

SEQ ID NO: 450 - DNA, 20 bp, Homo Sapiens
taaaccctaccccaagcagc

SEQ ID NO: 451 - DNA, 21 bp, Homo Sapiens
actcaaggcataaaagctggg

SEQ ID NO: 452 - DNA, 20 bp, Homo Sapiens
cctgacaaagtgcaggactc

SEQ ID NO: 453 - DNA, 21 bp, Homo Sapiens
gtgattttcgtggaagtgggt

SEQ ID NO: 454 - DNA, 20 bp, Homo Sapiens
tccaagctagtagtggccag

SEQ ID NO: 455 - DNA, 20 bp, Homo Sapiens
ggtcacacatgggtctgagg

SEQ ID NO: 456 - DNA, 19 bp, Homo Sapiens
caggccacacacacacttc

SEQ ID NO: 457 - DNA, 24 bp, Homo Sapiens
atagcccttacaacaaaaacaaga

SEQ ID NO: 458 - DNA, 20 bp, Homo Sapiens
gccagagcaggattaggaga

SEQ ID NO: 459 - DNA, 20 bp, Homo Sapiens
cagtgcgtgctcctttagtg

SEQ ID NO: 460 - DNA, 22 bp, Homo Sapiens
gctctgaaaatgctctttggga

SEQ ID NO: 461 - DNA, 26 bp, Homo Sapiens
tggacatttgtagaagaaataaggct

SEQ ID NO: 462 - DNA, 20 bp, Homo Sapiens
caccttcgcctttactgcag

SEQ ID NO: 463 - DNA, 22 bp, Homo Sapiens
attccttgaccacatcaaacct

SEQ ID NO: 464 - DNA, 20 bp, Homo Sapiens
agcacctcctactccctact

SEQ ID NO: 465 - DNA, 21 bp, Homo Sapiens
gacacccaaacaaggaactca

SEQ ID NO: 466 - DNA, 20 bp, Homo Sapiens
gagaccctctcttcagagcc

SEQ ID NO: 467 - DNA, 20 bp, Homo Sapiens
caatgatgctggtccacacc

SEQ ID NO: 468 - DNA, 21 bp, Homo Sapiens
aggagcttcatgacagactca

SEQ ID NO: 469 - DNA, 21 bp, Homo Sapiens
tgcagggcttcatacaagaga

SEQ ID NO: 470 - DNA, 19 bp, Homo Sapiens
ctcctcttctctggcagcc

SEQ ID NO: 471 - DNA, 20 bp, Homo Sapiens
ctgctcccctgtgaatcagt

SEQ ID NO: 472 - DNA, 20 bp, Homo Sapiens
agatcctgcacttgctcact

SEQ ID NO: 473 - DNA, 19 bp, Homo Sapiens
tccctccatgctcctctct

SEQ ID NO: 474 - DNA, 20 bp, Homo Sapiens
caaatatcagggtgcagggc

SEQ ID NO: 475 - DNA, 20 bp, Homo Sapiens
gattcatttctgctgggccc

SEQ ID NO: 476 - DNA, 20 bp, Homo Sapiens
ggtagtccagggtatgtggg

SEQ ID NO: 477 - DNA, 22 bp, Homo Sapiens
cacttatgcaaggagaatgctg

SEQ ID NO: 478 - DNA, 20 bp, Homo Sapiens
acaaaggaactgatgccctc

SEQ ID NO: 479 - DNA, 20 bp, Homo Sapiens
cctccccacatccatggtac

SEQ ID NO: 480 - DNA, 18 bp, Homo Sapiens
ctgacgagggcacacaga

SEQ ID NO: 481 - DNA, 20 bp, Homo Sapiens
attctcccattgcacagcct

SEQ ID NO: 482 - DNA, 20 bp, Homo Sapiens
ttctgtcatccatgctcccc

SEQ ID NO: 483 - DNA, 20 bp, Homo Sapiens
aagctacagccaggtcactt

SEQ ID NO: 484 - DNA, 20 bp, Homo Sapiens
tacctggaggatgatggctg

SEQ ID NO: 485 - DNA, 20 bp, Homo Sapiens
atctgggaccaagagtagcc

SEQ ID NO: 486 - DNA, 19 bp, Homo Sapiens
ctgaatgaggcagggaagc

SEQ ID NO: 487 - DNA, 20 bp, Homo Sapiens
acatccccgtgtcactactg

SEQ ID NO: 488 - DNA, 20 bp, Homo Sapiens
attccctgcacttctaggca

SEQ ID NO: 489 - DNA, 23 bp, Homo Sapiens
cagagtcacaaaataacacccca

SEQ ID NO: 490 - DNA, 20 bp, Homo Sapiens
gccaggtgaaagcacacgta

SEQ ID NO: 491 - DNA, 18 bp, Homo Sapiens
atctacctgccctgcacg

SEQ ID NO: 492 - DNA, 18 bp, Homo Sapiens
cacaggctgtggaggtcc

SEQ ID NO: 493 - DNA, 20 bp, Homo Sapiens
cactagagtggtgcagccta

SEQ ID NO: 494 - DNA, 19 bp, Homo Sapiens
gtaccagccccaagtggat

SEQ ID NO: 495 - DNA, 20 bp, Homo Sapiens
gtctcccattctctgcctct

SEQ ID NO: 496 - DNA, 20 bp, Homo Sapiens
ctgactggggtccacaaact

SEQ ID NO: 497 - DNA, 20 bp, Homo Sapiens
cattcccagctaccctcctc

SEQ ID NO: 498 - DNA, 24 bp, Homo Sapiens
tcttaaagcaattaaggagcacct

SEQ ID NO: 499 - DNA, 19 bp, Homo Sapiens
cagcattgtcctcaggcac

SEQ ID NO: 500 - DNA, 21 bp, Homo Sapiens
ataagagcatgaccctgcatg

SEQ ID NO: 501 - DNA, 19 bp, Homo Sapiens
caggagagtgtgcactggg

SEQ ID NO: 502 - DNA, 19 bp, Homo Sapiens
acaggtgtccccagagatg

SEQ ID NO: 503 - DNA, 20 bp, Homo Sapiens
ctgaggcagggcatgatact

SEQ ID NO: 504 - DNA, 19 bp, Homo Sapiens
cacctcacctgagtcccag

SEQ ID NO: 505 - DNA, 18 bp, Homo Sapiens
ccgccacgagctcagaag

SEQ ID NO: 506 - DNA, 20 bp, Homo Sapiens
tctccccatgacagccattt

SEQ ID NO: 507 - DNA, 20 bp, Homo Sapiens
ctcctcaggcatgggttctt

SEQ ID NO: 508 - DNA, 18 bp, Homo Sapiens
gtggcaagtggctcctga

SEQ ID NO: 509 - DNA, 20 bp, Homo Sapiens
ccctaccctgcctcaacata

SEQ ID NO: 510 - DNA, 24 bp, Homo Sapiens
aagtatacccaaccattcaactct

SEQ ID NO: 511 - DNA, 20 bp, Homo Sapiens
tacattctcccaaccccacc

SEQ ID NO: 512 - DNA, 20 bp, Homo Sapiens
tggtgatgtgcagtcaacag

SEQ ID NO: 513 - DNA, 18 bp, Homo Sapiens
ggcctgtctgtgtgctca

SEQ ID NO: 514 - DNA, 19 bp, Homo Sapiens
gagccacccacttcaggag

SEQ ID NO: 515 - DNA, 20 bp, Homo Sapiens
agggtgggtggaagtttagt

SEQ ID NO: 516 - DNA, 19 bp, Homo Sapiens
gagacacggttgggagagg

SEQ ID NO: 517 - DNA, 20 bp, Homo Sapiens
cccacctacacattccctca

SEQ ID NO: 518 - DNA, 20 bp, Homo Sapiens
cagcttcctatgcccagagg

SEQ ID NO: 519 - DNA, 18 bp, Homo Sapiens
ctgagcgtggtggcagtc

SEQ ID NO: 520 - DNA, 18 bp, Homo Sapiens
ctgtccctgctcgtcaca

SEQ ID NO: 521 - DNA, 20 bp, Homo Sapiens
cgtacccagaagacaatggc

SEQ ID NO: 522 - DNA, 24 bp, Homo Sapiens
ggctttgaaatggaactgaaaact

SEQ ID NO: 523 - DNA, 20 bp, Homo Sapiens
ccaagagtccccacatctgg

SEQ ID NO: 524 - DNA, 20 bp, Homo Sapiens
atggggttctccttgggaag

SEQ ID NO: 525 - DNA, 20 bp, Homo Sapiens
ggggtagccaggaagatctc

SEQ ID NO: 526 - DNA, 20 bp, Homo Sapiens
ctgcagcacttctttgtcca

SEQ ID NO: 527 - DNA, 19 bp, Homo Sapiens
gtgagggagggaaaggcag

SEQ ID NO: 528 - DNA, 20 bp, Homo Sapiens
tacacctcccctcttggaac

SEQ ID NO: 529 - DNA, 20 bp, Homo Sapiens
ctgaggggcaatagggaagg

SEQ ID NO: 530 - DNA, 20 bp, Homo Sapiens
gcactcggcagatctcagta

SEQ ID NO: 531 - DNA, 20 bp, Homo Sapiens
tcatcccttccatcacctcc

SEQ ID NO: 532 - DNA, 20 bp, Homo Sapiens
gagccctgttcttgctgatc

SEQ ID NO: 533 - DNA, 20 bp, Homo Sapiens
gcgccctatgaccttcacta

SEQ ID NO: 534 - DNA, 21 bp, Homo Sapiens
cagagtgtgcctaggaagttg

SEQ ID NO: 535 - DNA, 20 bp, Homo Sapiens
attcgcccgtagattgaccc

SEQ ID NO: 536 - DNA, 18 bp, Homo Sapiens
ccacagggacaagggctg

SEQ ID NO: 537 - DNA, 20 bp, Homo Sapiens
tgggattcgaaccaccaaac

SEQ ID NO: 538 - DNA, 19 bp, Homo Sapiens
gacaccaacccgtctaccc

SEQ ID NO: 539 - DNA, 20 bp, Homo Sapiens
ccaccttgcatgggtactca

SEQ ID NO: 540 - DNA, 20 bp, Homo Sapiens
acaaccttaggttccaagcc

SEQ ID NO: 541 - DNA, 19 bp, Homo Sapiens
gaggccccaggaagaatcc

SEQ ID NO: 542 - DNA, 19 bp, Homo Sapiens
ccctgcctctgtgtgtctg

SEQ ID NO: 543 - DNA, 19 bp, Homo Sapiens
ccatgcctggaactgcttc

SEQ ID NO: 544 - DNA, 18 bp, Homo Sapiens
catctccaccacccaggg

SEQ ID NO: 545 - DNA, 19 bp, Homo Sapiens
ctgaggctggccacttgaa

SEQ ID NO: 546 - DNA, 19 bp, Homo Sapiens
ccaaaggccaaaggaggtc

SEQ ID NO: 547 - DNA, 18 bp, Homo Sapiens
aaggctgcctgggacatc

SEQ ID NO: 548 - DNA, 19 bp, Homo Sapiens
tccccttccctgactccag

SEQ ID NO: 549 - DNA, 18 bp, Homo Sapiens
gacacaggctggagctcc

SEQ ID NO: 550 - DNA, 18 bp, Homo Sapiens
ccagggggagaaggacct

SEQ ID NO: 551 - DNA, 20 bp, Homo Sapiens
ccaagccgcttaatccttcc

SEQ ID NO: 552 - DNA, 20 bp, Homo Sapiens
tgcccccagaatgacaacta

SEQ ID NO: 553 - DNA, 20 bp, Homo Sapiens
aatctcagaccccacccttc

SEQ ID NO: 554 - DNA, 20 bp, Homo Sapiens
cccaccttgaacacgcaaat

SEQ ID NO: 555 - DNA, 20 bp, Homo Sapiens
cctcctctactgctaaggcc

SEQ ID NO: 556 - DNA, 20 bp, Homo Sapiens
gaccgcaaagccggtactta

SEQ ID NO: 557 - DNA, 20 bp, Homo Sapiens
cacccttttcccgtctgaag

SEQ ID NO: 558 - DNA, 19 bp, Homo Sapiens
gacctcagaccgaagtccc

SEQ ID NO: 559 - DNA, 18 bp, Homo Sapiens
agggaagggtgcaggtag

SEQ ID NO: 560 - DNA, 19 bp, Homo Sapiens
atatgtggggagcatgcgt

SEQ ID NO: 561 - DNA, 19 bp, Homo Sapiens
gctccaggcctttgtctta

SEQ ID NO: 562 - DNA, 20 bp, Homo Sapiens
tcaaaggcggccaaagaatt

SEQ ID NO: 563 - DNA, 20 bp, Homo Sapiens
cccactttctccccctcaat

SEQ ID NO: 564 - DNA, 19 bp, Homo Sapiens
ttctacacgaccaggccag

SEQ ID NO: 565 - DNA, 18 bp, Homo Sapiens
cctgaaccctggaccctg

SEQ ID NO: 566 - DNA, 18 bp, Homo Sapiens
cgaagtcctgggagcccc

SEQ ID NO: 567 - DNA, 20 bp, Homo Sapiens
tcggatggctacagtctgtg

SEQ ID NO: 568 - DNA, 20 bp, Homo Sapiens
cagacctgggtggctatgag

SEQ ID NO: 569 - DNA, 18 bp, Homo Sapiens
cagcaagcctggccatgg

SEQ ID NO: 570 - DNA, 18 bp, Homo Sapiens
tcctccccaactcccact

SEQ ID NO: 571 - DNA, 20 bp, Homo Sapiens
ccagagagtagaacagggca

SEQ ID NO: 572 - DNA, 19 bp, Homo Sapiens
ctggcctcacactgtctgg

SEQ ID NO: 573 - DNA, 20 bp, Homo Sapiens
gtaaaaactgcacccagcct

SEQ ID NO: 574 - DNA, 18 bp, Homo Sapiens
tcacccctaggcccatga

SEQ ID NO: 575 - DNA, 18 bp, Homo Sapiens
agtctccggagccccatg

SEQ ID NO: 576 - DNA, 19 bp, Homo Sapiens
cctctatatccccgccccc

SEQ ID NO: 577 - DNA, 18 bp, Homo Sapiens
cacctgtgcccgctccta

SEQ ID NO: 578 - DNA, 18 bp, Homo Sapiens
agggttccgtggggactc

SEQ ID NO: 579 - DNA, 18 bp, Homo Sapiens
taccaggcagggttggtg

SEQ ID NO: 580 - DNA, 18 bp, Homo Sapiens
cgtcgttgtctccccgaa

SEQ ID NO: 581 - DNA, 19 bp, Homo Sapiens
ctgcttccccaccatcctg

SEQ ID NO: 582 - DNA, 20 bp, Homo Sapiens
gtatgggctcagctgcaatt

SEQ ID NO: 583 - DNA, 20 bp, Homo Sapiens
ttgtggggtaggacagtgac

SEQ ID NO: 584 - DNA, 20 bp, Homo Sapiens
ctcgacaaagcaacaggtcc

SEQ ID NO: 585 - DNA, 20 bp, Homo Sapiens
agtgcaaggtcacagaggtc

SEQ ID NO: 586 - DNA, 20 bp, Homo Sapiens
aaatgagcctctcagtgccc

SEQ ID NO: 587 - DNA, 19 bp, Homo Sapiens
ctctctgggggctgagact

SEQ ID NO: 588 - DNA, 18 bp, Homo Sapiens
cacccacgaaaacccacc

SEQ ID NO: 589 - DNA, 19 bp, Homo Sapiens
tctgcgaagtcctgggaag

SEQ ID NO: 590 - DNA, 20 bp, Homo Sapiens
taacttccatcagaggcgct

SEQ ID NO: 591 - DNA, 20 bp, Homo Sapiens
atgcatctaaagccccgaga

SEQ ID NO: 592 - DNA, 19 bp, Homo Sapiens
catcctcccccagtgtctg

SEQ ID NO: 593 - DNA, 18 bp, Homo Sapiens
cacacacagggcccactg

SEQ ID NO: 594 - DNA, 20 bp, Homo Sapiens
gacttttcgagggcctttcc

SEQ ID NO: 595 - DNA, 19 bp, Homo Sapiens
gggcagacggggaaactta

SEQ ID NO: 596 - DNA, 19 bp, Homo Sapiens
ctgtcacaggccaagggag

SEQ ID NO: 597 - DNA, 20 bp, Homo Sapiens
caggtctttaggaggagggg

SEQ ID NO: 598 - DNA, 19 bp, Homo Sapiens
ctctagctggccggtcttc

SEQ ID NO: 599 - DNA, 20 bp, Homo Sapiens
tttccaacccctccctactc

SEQ ID NO: 600 - DNA, 20 bp, Homo Sapiens
ttttacgcgtggaatgcaca

SEQ ID NO: 601 - DNA, 20 bp, Homo Sapiens
aagagggaaaagttgccact

SEQ ID NO: 602 - DNA, 18 bp, Homo Sapiens
ccctcgcctccctcactg

SEQ ID NO: 603 - DNA, 20 bp, Homo Sapiens
gcgtatgatggaggcgtagt

SEQ ID NO: 604 - DNA, 18 bp, Homo Sapiens
aggatggccgcagagatg

SEQ ID NO: 605 - DNA, 19 bp, Homo Sapiens
cagacctctcctccagcct

SEQ ID NO: 606 - DNA, 19 bp, Homo Sapiens
caaagcctcgcacacactc

SEQ ID NO: 607 - DNA, 20 bp, Homo Sapiens
gtaacgattgcccagtgctc

SEQ ID NO: 608 - DNA, 19 bp, Homo Sapiens
caccccagcgcactagtta

SEQ ID NO: 609 - DNA, 143 bp, Homo Sapiens
gctcatttttgttaatggtggctttttgtttgtttgttttgttttaaggtttttggattcaaagcataaaaaccattacaagatatacaatctgtaagtatgttttcttatttgtatgcttgcaaatatcttctaaaacaact

SEQ ID NO: 610 - DNA, 143 bp, Homo Sapiens
gaacaatgaataaaccattttgtctcattaaaattttagattattatgtagttggcagctgagaacaatacttagtggataccatcgaatagtacaacaggtaagtcctttttaaaaggtttatgttttcagtgaagataaga

SEQ ID NO: 611 - DNA, 160 bp, Homo Sapiens
atcatgctgacgcttctcctttatcttttaaaatttgcagtggctgagaggactactaactagtagacagagttttaacatcatatttggtgaatgtccatattgtagtaaggtaagcaaattgttaatcacatctcataaaatgtgcctagctttcagt

SEQ ID NO: 612 - DNA, 241 bp, Homo Sapiens
tgcagatttttaaaaagtctcttttccattattttttcaacttataggaatgaggcaaagtagcctaaagaaagattggttcttatcagaagaagaatttaaattatggaacagactttatagattaagggacagtgatgaaattaaagagataacattgcctcaagttcagttttcttctttacaaaatgaggaaaacaaaccagtaagttgaatatattttcagatgttcttttccccc

SEQ ID NO: 613 - DNA, 104 bp, Homo Sapiens
agttaataaggacatggtttctgttctttttttacagatacttttaaagttttgtcagaaaagagccactttcaaggtaggacaagtttggaaatgtattctca

SEQ ID NO: 614 - DNA, 173 bp, Homo Sapiens
aggaaactagagttcattttccttttcttaggacctgttatggaatttgattatgtaatatgcgaagaatgtgggaaagaatttatggattcttatcttatgaaccactttgatttgccaacttgtgataactgcaggtacttattttagatggtgttcttaattgctaatgt

SEQ ID NO: 615 - DNA, 209 bp, Homo Sapiens
tcgcaagtctgaatcacaacttatttaaatatggattttgtgttgtagattgtgaagaggtctcttgaagtttggggtagtcaagaagcattagaagaagcaaaggaagtccgacaggaaaaccgagaaaaaatgaaacagaagaaatttgataaaaaagtaaaaggtagatggccacattttatatcgtgaaggattgtgttttacca

SEQ ID NO: 616 - DNA, 192 bp, Homo Sapiens
tgtgcattgagagtttttatactagtgattttaaactataatttttgcagaatgtgaaaagctatttttccaatcatgatgaaagtctgaagaaaaatgatagatttatcgcttctgtgacagacagtgaaaacacaaatcaaagagaagctgcaagtcatggtaagtcctctgtttagttgaactacaggt

SEQ ID NO: 617 - DNA, 332 bp, Homo Sapiens
ccaggttagctggtaagaggatttttttggagaaaaaaatgatatttagaaagttaatttctaattccggaatggaataaaaacaatatgagtagtgtaatcttgtagaaaaagagttgtataatcttgtagaatttctcattctgtggtacaacccaggggtaaactattattccagtagtcagtacacttttctagataaatcttgagtgaaaaccagcaatttctttttccttgtggtctgattcctttttctaatccatgaaggccatcttgtagattacatttatcattaatgcaagaataaagacaattcctcctgtcagttgcgt

SEQ ID NO: 618 - DNA, 155 bp, Homo Sapiens
aagcttttcaggataatttctgtttttttttgtttgtttgtttttaggagttatggtacagcacctgtaaatcttaacatcaagacagggggaagagtttatggaactacaggtaaaatttgtattttctgttgcatcaatagggaaataaggca

SEQ ID NO: 619 - DNA, 212 bp, Homo Sapiens
aggatgatactggttgacacaattgttttattttatttcagttggttagaagcaaacgctgaatatcttgtagaaagagattatgaatcagcttgtaaaatatggagtggaaatgaaatgctcttaactttacacaaaatgggtatcaccactgctacttttcccattttgcaggtaagatattttttctacctgtgaaacgtatttagtga

SEQ ID NO: 620 - DNA, 151 bp, Homo Sapiens
tcagcttttccttaaagtcacttcatttttattttcagtgaagaactgttctaccagatactcatttatgattttgccaattttggtgttctcaggttatcggtaagtttagatccttttcacttctgaaatttcaactgatcgtttctga

SEQ ID NO: 621 - DNA, 150 bp, Homo Sapiens
caccatatattaacttctgacatttgcaaatttcaggagttgtcacagcagaaatgtttagacatatgcagaactctgagataattcgaaaaatgactgaagaattcgatgaggtaacttactaccttaagtgttttgataacttatgca

SEQ ID NO: 622 - DNA, 150 bp, Homo Sapiens
gctacaaaagagctcaacaatttttttattacgtgcaatttttttaataggaataaaaaaatacgttgttggcctcattatcaagacgtcatctgacccaacttgtgtagaggtaagagtttattttggagcttattgggagagcataaa

SEQ ID NO: 623 - DNA, 146 bp, Homo Sapiens
gtgattttctaaaatagcaggctcttatttttctttttgtttgtttgtagcgatacaaacttggagttcgcttgtattaccgagtaatggaatccatgcttaaatcagtaagttaaaaacaatataaaaaaatttcagccgggcgc

SEQ ID NO: 624 - DNA, 128 bp, Homo Sapiens
tcaccactgagcaaatgtttgaaattatgttaattttgacaggtacaaaatcaacatgcaaaagaagagtctcttgctgatgatctttttaggtaaagtttgattcacctttctcatgtacctttgag

SEQ ID NO: 625 - DNA, 199 bp, Homo Sapiens
agcagttgtgattctttctttctacttgtgtgatttacaggattaaagacaacaactccaggaccaagcctttcacaaggcgtgtcagttgatgaaaaactaatgccaagcgccccagtgaacactacaacatacgtagctgacacagaatcagagcaagcagatacatggtaaagcttcttcattaccgtactattgt

SEQ ID NO: 626 - DNA, 160 bp, Homo Sapiens
ttggaagtataatagcagttcttttctctttataggttagaccaaagccattgcttttgaagttattaaagtctgttggtgcacaaaaagacacttatactatgaaagaggtaagctgaatcaagagataagtagtatctcactagttacatgtagccat

SEQ ID NO: 627 - DNA, 177 bp, Homo Sapiens
tgacagctatgctaatagtgtttcttgtctatataattgcagatctctacaatatggaaatcagttcatatatcagtcaatgccacgaatgttaactctatggcttgattatggtacaaaggcatatgaatgggaaaaaggtataactcttcacgtaaaagttttaaaggactagga

SEQ ID NO: 628 - DNA, 159 bp, Homo Sapiens
tgggtgcttgggtgtgtataaacacatgatactgtatcatagcaataagatttgtaaaacattgcaatggctgaaaaactgtctaacaggaaattttaagtgttattccaaagaagaacaagcctaaaagtaagttaacctttcagagtggttgaaagt

SEQ ID NO: 629 - DNA, 176 bp, Homo Sapiens
tctgtcttctcctctcttcctttacacaaacttaaacagaatggaaatgaaaaccaaggagaagttgaagaacaaacatttaaagaaaaggaattagacagaaaacctgaagatgtgcctcctgagattttgtctaatgaaaggtatacaaaatgtgtactttttcagtgcaaagt

SEQ ID NO: 630 - DNA, 150 bp, Homo Sapiens
tggttaggagaatgagttgttgttgttgatgttgttttttaaccacacagacctttccaaataaaagattccagttgcatatgaaatattagatcacaagtacagtaagtaatatttctctaacatgtcatccctcttgaaggagctgtc

SEQ ID NO: 631 - DNA, 152 bp, Homo Sapiens
tgcatctgtggcattaaatggtgatacatattatttgaatttcagatttacggcaagatatgctaacacttcaaattattcgtattatggaaaatatctggcaaaatcaaggtcttgatcttcggtaggtaaccagtaaggcaacctgtatg

SEQ ID NO: 632 - DNA, 108 bp, Homo Sapiens
atggtttttatgacacagagttgtgattttttttctttttcacagtttctcaagcaagacctcccccaaatcagaagaaaggtgaggttgtgcatatgattttaaact

SEQ ID NO: 633 - DNA, 161 bp, Homo Sapiens
aggatcactttatatggatcactttttcttattttgtagtcttcaaaaattaaaaaaggaagcaaaggggatacatccatgttgaaaccaacactgatggcagcagttccggtaagaagtgacccttatttaatattgagtattaaagaagtatcacccgc

SEQ ID NO: 634 - DNA, 158 bp, Homo Sapiens
tgtgtacatggtgccagaatatttgtttttcttcttatagaatgtccaacatcccagcttgcttccttggatcagcacagtcagaaaatgttctaacagatattaaattgtgagtaatttttttccctcaacttttattttggatttatgggggtgca

SEQ ID NO: 635 - DNA, 200 bp, Homo Sapiens
agtcaatttttctttcttctcttgcaggtgaacctatgggtcgtggaacaaaagttatcctacacctgaaagaagaccaaactgagtacttggaggaacgaagaataaaggagattgtgaagaaacattctcagtttattggatatcccattactctttttgtaagtttttatgtaattgcagagtgaatttctgtctgt

SEQ ID NO: 636 - DNA, 150 bp, Homo Sapiens
ttggaggccactgctatggcttatgaaaataattgttttttgttttacagtgggcaaatcaaactagcagattttggacttgctcggctctataactctgaagagaggtaaggcattaattaaaattacatgtgggaaataaggtttgtt

SEQ ID NO: 637 - DNA, 194 bp, Homo Sapiens
aaatgtattcactgtcttctctttctttaggttccagcaacaaattatatatatacacccctgaatcaacttaagggtggtacaattgtcaatgtctatggtgttgtgaagttctttaagcccccatatctaagcaaaggaactggtaggtattaaaactggtggagttttttggagtggaaatgcattattga

SEQ ID NO: 638 - DNA, 154 bp, Homo Sapiens
tgtgtgactctaaattgactgcattaattttctcactctcgtcttgcagcattttcagttagctttggttgactgtaatccctgcactttgtccaatgctgaaagtaagtattattaagtactgtagttttctacatgtattccgatcacttca

SEQ ID NO: 639 - DNA, 211 bp, Homo Sapiens
tgtgtgacatgttctaatatagtcacattttcattatttttattataaggcctgctgaaaatgactgaatataaacttgtggtagttggagctggtggcgtaggcaagagtgccttgacgatacagctaattcagaatcattttgtggacgaatatgatccaacaatagaggtaaatcttgttttaatatgcatattactggtgcaggacc

SEQ ID NO: 640 - DNA, 183 bp, Homo Sapiens
atgcaaagtacctaactccactgatttctttttccctcactttttaggattatggctgctgttcctcaaaataatctacaggagcaactagaacgtcactcagccagaacacttaataataaattaagtctttcaaaaccaaaattttcgtaagtgttttgactggtttgctgtcacataggc

SEQ ID NO: 641 - DNA, 161 bp, Homo Sapiens
tgtttgtgcctcactttgcaggagttccactatatagaagaagatctttatcgaacaaagaacacattgcaaagcagaattaaagatcgagacgaagaaattcaaaaactcaggaatcaggtatgaatcactattcacaacttgtgaaaagatgccactga

SEQ ID NO: 642 - DNA, 150 bp, Homo Sapiens
acacgtcttggggatatcacttttgttatttttatttttagggaagcacactgagaaagcggaagatgtatgaggaattccttagtaaagtctctattttaggtgagttgtaaagtgtgttaactttgctagtatgtgagatacccctga

SEQ ID NO: 643 - DNA, 155 bp, Homo Sapiens
actggtatggaaatattacagtcctgtaattctttcttctaggtcatattgaacattccagatacctatcattactcgatgctgttgataacagcaagatggctttgaactcagtaagtggttaattattaccttcctggccttttctttgttcc

SEQ ID NO: 644 - DNA, 198 bp, Homo Sapiens
gccatgttttacatttgagttcacagaaaggaaatgtttattctaggtctgcttcgcctgtgtagatgggaaagaattccgtcttgctcagatgtgtggacttcatattgttgtacatgcagatgaattagaagaacttatcaactactatcaggtattaacgagacttttatatgacctgagatcttttaccataga

SEQ ID NO: 645 - DNA, 160 bp, Homo Sapiens
acaaggctgagacctgagttgataaaatttctttgttctttcagtgaagagaaaggaagtacagaaaacatgcagaaagcacagaaaggaaaaccaaggttctcatgaatctccaactttaaatcctgtaggtattgaaataggtatcagctttccttga

SEQ ID NO: 646 - DNA, 173 bp, Homo Sapiens
tgtcactttgttctgtttgcaggtggaaaaccatgaattccttgtaaaaccttcatttgatcctaatctcagtgaattaagagaaataatgaatgacttggaaaagaagatgcagtcaacattaataagtgcagccagagatcttggtaagaatgggtcattggaggttggaa

SEQ ID NO: 647 - DNA, 135 bp, Homo Sapiens
tggctgttgtcactattgcatatgctaactttttctgtttacatttcagggcatttgaatatgaaattcgattttacactggaaatgaccctctggatgtttgggataggtgggtctttttatttcacaaggaca

SEQ ID NO: 648 - DNA, 150 bp, Homo Sapiens
gctgtttctcttttctccaccattctataggaataagatggtagaatacctgacagactgggttatgggaacatcaaaccaagcagcagatgatgatgtaaaatgtcttacaaggtaaaaaaagaatgaccttcaagtattagtgggttt

SEQ ID NO: 649 - DNA, 150 bp, Homo Sapiens
actgtgtattcatgttcccctttctagtgctgattataaacctaagaaaattaaaacagaagataccaagaaggagaagaaaagaaaactagaagaagaagaggttagtaaagagacttaggtcctttggggcttgagtttggaagtggg

SEQ ID NO: 650 - DNA, 150 bp, Homo Sapiens
ggatttaactttgtttttctctgcctgattgggttctctctttattttagtttctacagagcctgtggagacctctcgatggacagaagaagaaatggaagttgctaaaaaaggtaaattgtagtagttctggtttctcaggtttttgtt

SEQ ID NO: 651 - DNA, 158 bp, Homo Sapiens
agacttcgagtgtttaaatgaaagattaaagtctcaatacttttttaggtacaatggtggctgttgcaattagcccaggaactagaggagagactgactaaagaccgaaatgatgtaagattttctttctttattcctggggtgggctttgcaattta

SEQ ID NO: 652 - DNA, 128 bp, Homo Sapiens
tcagaagttctggaaatacttcatttatttttaaatccttttgttttaggctctccagcagaactatctcctactactctttcccctgttaatcatagcttgggtaagttgcacatatgttcccctca

SEQ ID NO: 653 - DNA, 152 bp, Homo Sapiens
atgcttcagatgttgtatgtaatgtattctttttattttatgtgtagatggctcttggacaagtccgagcagtacaacacactgggagggaatgccctctccttttaaaggcaggaatttttatttattacctgtgttcagtatgtaaacgt

SEQ ID NO: 654 - DNA, 133 bp, Homo Sapiens
ctactcatgcccctcaaacttatttttaataatttcttttcccttcacagctatttgcatgcaaagaacatcatccatagagacatgaaatccaacagtatcctttggttgttgagttcatttgactgctcgg

SEQ ID NO: 655 - DNA, 151 bp, Homo Sapiens
tcttaaatgatcctcttctcccagataatagatgccactcaaaaaggaaattgctctcgtttcatgaatcacagctgtgaaccaaattgtgaaacccaaaaagtaagttgaggtggatttagagtttgaggtatttgttcacgattgctgt

SEQ ID NO: 656 - DNA, 151 bp, Homo Sapiens
gcgatgtgcatatataatggaaaggtttttgttattttcagggtccaccaatgagctgtgtacgattggctgaaagagttgtagctctcatttgctgtgagaaactgcacaaaattggtaaggatgtttgaaaagcatatacgaatgaaaa

SEQ ID NO: 657 - DNA, 151 bp, Homo Sapiens
aatcctgtttgcagcgtgagttaacctgcaactgattttgttttacagatggttttatcattagcgtctgaactcagagagaatcatcttaatggatttaacactcaaaggcggtaggtgttaaactaaacatccttcttctcaggtttca

SEQ ID NO: 658 - DNA, 151 bp, Homo Sapiens
actccattcagaaagtaattttcacctttttttttttttcaaggaaactgagaagaaaatatggcacttcaaatatcctatcttcttcctgtgtatagggctagacttacaggtagagtgaagctatgggaccaggtcacagtgaagtcct

SEQ ID NO: 659 - DNA, 171 bp, Homo Sapiens
tcaggatttttcctttctctctcctattattagacttattcgtctaatggaagagatcatgagtgagaaggagaataaaaccattgtttttgtggaaaccaaaagaagatgtgatgagcttaccagaaaaatgaggagagatgggtatgtgtgagctcctccattgaagca

SEQ ID NO: 660 - DNA, 339 bp, Homo Sapiens
gagttctaggctactgtggctttttccagtagatttagatgagattatgtgttttgaaatgttttgtgggatcccttagaaagcatcacttcagggcagagacactcaatattgccagccagcttgggttctaaagtgatttaatcaaattcatgctcctgatcttttttttcccccttcctttggctatgaaaacccaaagcccggagtgattgttttctccttgctttaagcagtgaagttatcctaatgcaaaagagcttagtagaaaatgagtggtttacctttttttctaaaagtatattttcaagtttattctggaatgtgatgtcttggtcc

SEQ ID NO: 661 - DNA, 176 bp, Homo Sapiens
gtgaatacactttctgctggttttaaatgacagatactgaaataaaaataaacatcaaacaagaaagtgcagatgtaaatgtgattggaaacaaggatgtcgttactgaagaggatttggatgtttttaagcaggcccaggaactttcttgggaggtaaggcgaggatcccacatt

SEQ ID NO: 662 - DNA, 159 bp, Homo Sapiens
aaccaagtcagtaaggccatatacagttattatgttttttactctcaggggaaagttggggacatgctgctacaatacggctaatctttcattgggaccgaaagcaaaggtcagtacagaaacaagttaataactccgaatattgggttaattatactg

SEQ ID NO: 663 - DNA, 151 bp, Homo Sapiens
aaacccttctgtttctttcacagatgagtgccaaaccaaatatggaaatgctaatgcctggagatactgtaccaaagtttttgacatgctcacagtagcagctgtaagtttctatttttaaagtctcatgtacgttcctgccaataacaca

SEQ ID NO: 664 - DNA, 164 bp, Homo Sapiens
tcctgtgagtgttctgtatgttaacacttttattccttgttttgttttaggcgataaactacattcagttgagtctgcaagactgggaggaactggggtgataagaaatctattcactgtcaaggtgagaattagcaaattttcccccttcttctgtactttct

SEQ ID NO: 665 - DNA, 161 bp, Homo Sapiens
gctgggaacatttgtcatttattcttttctactccttgtattttgtgcaggtctgcagactcgtgaatgaggtctaccacatgtataatcgacaccagtatccatttgttgttcttaacatttctgttgattcaggtaagttccattggcatttcagtaca

SEQ ID NO: 666 - DNA, 124 bp, Homo Sapiens
tgccgtttcgagaattttattcacttttatatttatgtctcacatctagggaactagctcttcagaaaaatccaagtcttcaggatcgtcacgatcaaagaggttggttgatgttgaagggaca

SEQ ID NO: 667 - DNA, 155 bp, Homo Sapiens
tgcacactgaaattaggacgtttatatttcttcaggtattagaaaactactcggatgctccaatgacaccaaaacagattctgcaggtcatagaggcagaaggactaaaggaaatgaggtttgtattgttcttgttgcttaacatgagggtttca

SEQ ID NO: 668 - DNA, 150 bp, Homo Sapiens
tttttcccccttgatccttctaggtggggacggatggcctgttgcgtctcagcagcagtgcactaaataacgagttttttacccatgcggctcagagctggcgggagcgcctggctgatggtatgtagacttggtcatctcggacggctt

SEQ ID NO: 669 - DNA, 152 bp, Homo Sapiens
acttattgagcatgcctggttttttgtcttccagcaatatccagattattatgcaataattaaggagcctatagatctcaagaccattgcccagaggatacaggtatgaagatgaccataagtaaatgattgtggtttagagcagattggcc

SEQ ID NO: 670 - DNA, 162 bp, Homo Sapiens
acgcagtgctaaccaagttctttcttttgcacagggcattttggttgtgtatatcatgggactttgttggacaatgatggcaagaaaattcactgtgctgtgaaatccttgaacagtaagtggcattttatttaaccatggagtatacttttgtggtttgca

SEQ ID NO: 671 - DNA, 100 bp, Homo Sapiens
ttttatatgatctttcctggttggcaggacccatggatgaaggaccagatcttgatctaggtaattttgaattctagttgtgcttcatatcgtgctttgt

SEQ ID NO: 672 - DNA, 163 bp, Homo Sapiens
aagaatgcctgggactgaggggagatatttttgtttgtcagagtcagagcactttttccgatgctgtttggataaaaaatcacaaagaacaatgcttgctgttgtggactacatgagaagacaaaagaggtaatgtaatgagtgttgcttcttacgtttagga

SEQ ID NO: 673 - DNA, 150 bp, Homo Sapiens
aaagtgtagactaatgatgtgacttttgttttcacagactgaaacagcagagcttcctgcttctgatagcataaacccaggcaacctacaattggtttcagagttaaaggtcagaagaatattctcttccagtgtctcgtgtcttacata

SEQ ID NO: 674 - DNA, 150 bp, Homo Sapiens
tgtactctttttcctgttgctgttcttttctgcaggagggaaaacccctgatcctaaaatgaatgctaggacttacatggatgtaatgcgagaacaacacttgactaaagaagaagtatgtaaacctgtctcctgttttaatggttgtga

SEQ ID NO: 675 - DNA, 151 bp, Homo Sapiens
aagcaaacttgttctgtttttacccactgattctttttcagccccttctcaaagtcagcatgtcaatgagagactgcttgatacttgtccttcggaaagctatgtttgccaagtatgtagcatctttttctatcataggaagacgttgtct

SEQ ID NO: 676 - DNA, 161 bp, Homo Sapiens
tgtgtgtttctgtgggtttctttaaggtttggacagaagggtaaagctattaggattgaaagagtcatctatactggtaaagaaggcaaaagttctcagggatgtcctattgctaagtgggtaagtgtgacttgataaagcctttggtcttaaatcttggg

SEQ ID NO: 677 - DNA, 150 bp, Homo Sapiens
tgtttttgtccctctctcttgcagtgaaattacagacaacccttacatgacgtcaatccctgtgaatgcttttcagggactatgcaatgaaaccttgacactgtgagtattaccagttctactccctccatcaactaaattctattttga

SEQ ID NO: 678 - DNA, 169 bp, Homo Sapiens
acatactttgtatcatcttattggcagcactggaagaagctgcaaaacgtttccaggaattgaaagcacaaagagaaagtaaagaagccctagagattgaaaaaaactcaagaaaaccccctccctacaaacacatcaaagtaagtctttatctctggcctgggattgc

SEQ ID NO: 679 - DNA, 159 bp, Homo Sapiens
tgttttcctttgtgtcattcccttttatcaggttgcccacattcccaaatcagatgctttgtactttgatgactgcatgcagcttttggcgcagacattcccgtttgtagatgacaatgaggtgaggtataaaataacctggttaatagaaaaactcca

SEQ ID NO: 680 - DNA, 166 bp, Homo Sapiens
ggattcagtgaggaatgctttcatgaagttggtgtatctttttaaataggaaaatccttgtaaatgatggtgacgcttcaaaagccagactggaactgagggaagagaatcccttgaaccacaacgtggtaagagattaatagcttctgttgatgccatatctgca

SEQ ID NO: 681 - DNA, 163 bp, Homo Sapiens
acacagaatttgttaagcaacgtagtctttcttttaaattctctttcaggtttttcatatgaaaaagatccccgattatactttgacgacacttgtgttgtgcctgagagactggaaggtaagtagccccatccaaggtttgttgccaggtagaggtttggag

SEQ ID NO: 682 - DNA, 133 bp, Homo Sapiens
tctcatcataccactattattttgcagttttgtgtgcgacaactgcttgaagaaaactggcagacctcgaaaagaaaacaaattcagtgctaagagtaagtttcgggaagctttctgtttcctggactgcaca

SEQ ID NO: 683 - DNA, 182 bp, Homo Sapiens
acacagccttctcaacctctttttctttttttctttcttgtttttagtctttttgctaaagaacatctgcagcacatgacagaaaagcagctgaacctctatgaccgcctgattaacgagcctagtaatgactgggatatttactactgggccacaggtactgggtatgataagcagcataa

SEQ ID NO: 684 - DNA, 150 bp, Homo Sapiens
ctctcggtgtatttctctacttacctgtaataatgcttttgtcttaatagggtggttctcttcccaaagtggaagccaaattcatcaattatgtgaagaattgcttccggatgactgaccaagaggtaactggattttctggggacatga

SEQ ID NO: 685 - DNA, 150 bp, Homo Sapiens
tcactgtgatagccttatctgcttgtttctctttgactttgtagctcgttctccggtttttagtgccatgtttgaacatgaaatggaggagagcaaaaaggtatgtaacaagatgaagacatgtctgtttagccaggtatctagagtcag

SEQ ID NO: 686 - DNA, 150 bp, Homo Sapiens
gttaatcttaacctgtgctttgcctcctgttctgtcttgactttgccagaaaaatcgagttgagcagcaacttcacgagcatttgcaagatgcaatgtccttcttaaaggatgtctgtgaggtactatttcttttagatggtgccttttt

SEQ ID NO: 687 - DNA, 120 bp, Homo Sapiens
actccaacactgttgctcctaattactgttttatcctacttttaggactctggagatgccacatggaaagaaacattctggttggcaagtatcttcttacatgctcttacttgctccaag

SEQ ID NO: 688 - DNA, 122 bp, Homo Sapiens
gccagagtcctttagccctactcaggttaaaatgatgttttgtttttcagttacttacacgccaagtcaatcatccacagagacctcaagagtaatagtatccttcctgaaatttgtctgcg

SEQ ID NO: 689 - DNA, 229 bp, Homo Sapiens
tcctgggtttcttttcaacttgtaatagtgttgtattcttgtctttaggcaagccaaaattccttccggatagaatatgatacctttggtgaactaaaggtgccaaatgataagtattatggcgcccagaccgtgagatctacgatgaactttaagattggaggtgtgacagaacgcatgccagtaagtggcatttgtggaaatgttggctattttggatgaagtaggc

SEQ ID NO: 690 - DNA, 182 bp, Homo Sapiens
tcctgaagtgagatcttacatcctttcttctcataggtgaagagaaggcagagaaaggacttagcgtccagtgactccaggaaaacgacagagaagcttctgaaaacatttttgaaaagacaagccatcaaaactgccttcagaagcaaaaggcaagaggaaaattatagtcgtgttagaga

SEQ ID NO: 691 - DNA, 150 bp, Homo Sapiens
gaccagaaaggctcagttccctgttttctcttcctaacattttagcaagaacagtgatgaaatcaacatacctcgactcattgtcagtcaactaaaatggcttgacagagttgtggatggcaaggtaggcttatggactttatctcttga

SEQ ID NO: 692 - DNA, 150 bp, Homo Sapiens
aaacattgatttggcttttcccatttattattctgtaggttgctatgacccgagagaagtttccagagaagattccatttagactaacaagaatgttgaccaatgctatggaggtgagtggatatcgggaacgagctgcttccaaatggg

SEQ ID NO: 693 - DNA, 176 bp, Homo Sapiens
tgtctccatttcccaccacagggtcatgctctatcagatttcagaagaagtgagcagatcagaattgaggtcttttaagtttcttttgcaagaggaaatctccaaatgcaaactggatgatgacatggtaagacctggtatcttactgagatttagtcctgagacttggttagcca

SEQ ID NO: 694 - DNA, 156 bp, Homo Sapiens
atttactctggattttgctttttcagatactggctttggcactactagtggaggggcatttggaacatctgcatttggttctagcaacaatactggaggcctctttggaaattcacagactaaaccaggtaggggctttatttgtgatataactgg

SEQ ID NO: 695 - DNA, 151 bp, Homo Sapiens
tgagatgtttttgccttcacaggtgattgaagtgggaaaaaatgatgacctggaggactctaagtccttaagtgatgataccgatgtagaggttacctctgaggtatgaatctttagcaagaactattttgcaccagccccatcttcagat

SEQ ID NO: 696 - DNA, 165 bp, Homo Sapiens
tcccccttgtcttaaaccacaggatttattggatcattcgtgtacatcaggaagtggctctggtcttccttttctggtacaaagaacagtggctcgccagattacactgttggagtgtgtcggtaattcttttttttcctttctttgtgggtaatatgcaatgtt

SEQ ID NO: 697 - DNA, 153 bp, Homo Sapiens
ttgacgtgccattctcttgcttcctcttcctcaaaaaggataagcactgttcaaaggatgccctacttgcaggattaaaacaagatgaaccaggacaagcaggaagtcagaagtcttctaccaagtaaggaaaatgagtttacttcctgttgt

SEQ ID NO: 698 - DNA, 150 bp, Homo Sapiens
tttgggacgacttattgcccttcaatttcctcatggccaaaggcttattcatagggcttcttgactaaagcccttggagcactgggtttttcttgaagtatatggtgagttaatgacttgaatctgcaattgggtgattgcctgaattct

SEQ ID NO: 699 - DNA, 157 bp, Homo Sapiens
agagtaacttcaatgtctttattccatcttctctttagggtcggattccagttaaatggatggcaattgaatccctttttgatcatatctacaccacgcaaagtgatgtgtaagtgtgggtgttgctctcttggggtggaggttacagaaacaccct

SEQ ID NO: 700 - DNA, 264 bp, Homo Sapiens
gatctccggttgggattcctgcggattgacatttctgtgaagcagaagtctgggaatcgatctggaaatcctcctaatttttactccctctccccgcgactcctgattcattgggaagtttcaaatcagctataactggagagtgctgaagattgatgggatcgttgccttatgcatttgttttggttttacaaaaaggaaacttgacagaggatcatgctgtacttaaaaaatacaagtaagttctctgcacaggaaattggt

SEQ ID NO: 701 - DNA, 159 bp, Homo Sapiens
gcattttctctgttcaacaagcagaacaggataattcagacaacaacaccatctttgtgcaaggcctgggtgagaatgttacaattgagtctgtggctgattacttcaagcagattggtattattaaggtacttgtggagaggagtgggagctttctgt

SEQ ID NO: 702 - DNA, 151 bp, Homo Sapiens
gtttgtgacataagcagaacgctttgatttggtttctttctacagctccattttcagtccggcagcacacattactctgcgtacaaaacgattgaacaccagattgcagttcaggtaggaaacgcaagagattctgaagcttgaattgtct

SEQ ID NO: 703 - DNA, 155 bp, Homo Sapiens
tgacagccagattcgtctttgttttatagagttgttgcctgcaatctctatccctttgtaaagacagtggcttctccaggtgtaactgttgaggaggctgtggagcaaattgacattggtaagtcagaaaaaccattttagaagactgagaggag

SEQ ID NO: 704 - DNA, 190 bp, Homo Sapiens
tggaagtctgtgagttctctgatctttacttcttttttaggcacacctgttgtccttaactgactttccgggtcatcagcacttcttcagaagtgttccttttccctcccagcaaatatggaagactgactatcctgatattcccagacattctttgctgtgatttgtaagcccgatataaacccttcca

SEQ ID NO: 705 - DNA, 150 bp, Homo Sapiens
atgttcttcctttaggtatagtctcaataacatttcttcccctaggtgctgctgatgtagagaaggtggaggaaaagtcagcaatagatctgacccctattgtggtagaagacaaaggtgggtgtttggagaagttgattgtggttcaga

SEQ ID NO: 706 - DNA, 152 bp, Homo Sapiens
cctgaagcagtccaggacttatgtgaccgtggtctctttttcttctagttgatcataccagggttgtcctacacgatggtgatcccaatgagcctgtttcagattacatcaatgcaaatatcatcatggtaagctttgcttttcacagtgtt

SEQ ID NO: 707 - DNA, 154 bp, Homo Sapiens
agcttaagaaaagtgacgtggtcaatttttttcttaaatagatcactttgccagctgtgtggaggatggatttgagggagacaagactggaggcagtagtccaggtgagagatgttgagatgttgcagatttgaactaaggcattggcagtaga

SEQ ID NO: 708 - DNA, 156 bp, Homo Sapiens
tccttgagtgtaaggcaattaataacttacacttgtctttatgttccagcctgaaagaatagacccaagcgcatcacgacaaggatatgatgtccgctctgatgtctggagtttggggatcacattggtatgtttatgctgattcaaccttgccac

SEQ ID NO: 709 - DNA, 151 bp, Homo Sapiens
gtctgtggattgacttcttttccttttatgttgttctggtttttaaaggcagcagcctccttggaaagacgaaaagcatcctgggttcaggctgacatctgcacttaacaggtacatgggttgtttcctggtaggagtagattttaaagca

SEQ ID NO: 710 - DNA, 125 bp, Homo Sapiens
tgagcctcatttattttctttttctccccccctaccctgctagtctggagttgatcaaggaacctgtctccacaaagtgtgaccacatattttgcaagtaagtttgaatgtgttatgtggctcca

SEQ ID NO: 711 - DNA, 150 bp, Homo Sapiens
tcccttatgttcttcttctctccagatctgttacctaaagcaataaaaaatggccagaggatcagtgtccgatgaggaaatgatggagctcagagaagcttttgccaaagttggtgagtagacctggtaccccaaattgcatcattctct

SEQ ID NO: 712 - DNA, 150 bp, Homo Sapiens
gcactttaatactaacaggaacagccttgaaaatgtcttttctttccagtacaccccaaaaagggagatttggtatactctgagatccagactactcagctgggagaagaagaggaaggtacgtctttgggaagagattctggctgcaaa

SEQ ID NO: 713 - DNA, 154 bp, Homo Sapiens
tgtcctcagtctgtactaaactcaaaccaagttctcatgcattactaggttggagaaacgtacggtaaggatataacctcccggggcaaagacaagccgattgccgtatgtaaaactttcagtccacttcagtttcaagctttctctgttctct

SEQ ID NO: 714 - DNA, 131 bp, Homo Sapiens
agcagatttgtatgaaagcccttacattttttctaggtatgaagtagctccgaggtctgatagtgaagaaagtggctcagaagaagaggaagaggtaagagtgcatttcctggctttcaaggctctcagtg

SEQ ID NO: 715 - DNA, 216 bp, Homo Sapiens
accctgttacacgcttgtaattgactcttctaggtgagcccattggcaggggtaccaaagtgatcctccatcttaaagaagatcagacagagtacctagaagagaggcgggtcaaagaagtagtgaagaagcattctcagttcataggctatcccatcaccctttatgtgagtatggacttttaaatcttttacacttaacgtgcaggatgtttcc

SEQ ID NO: 716 - DNA, 156 bp, Homo Sapiens
actgaatttgttttagggcacattgaatactttactttccttttcctcagcatcaacaacagcaacttgtgattggcggtgaccggatattcagttgcacatccccacatcaatgcactgccaatggtaagactctccaactcccaatgtcagtgt

SEQ ID NO: 717 - DNA, 213 bp, Homo Sapiens
caccggtgtggctctttaacaacctttgcttgtcccgataggtcacctttggctcttcagagatgcagggacacacgatgggcttctggttaaccaaactgaattatttgtgccatctctcaatgttgacggacagcctatttttgccaatatcacactgccaggtactgacgttttactttttaaaaagataaggttgttgtggtaagtaca

SEQ ID NO: 718 - DNA, 158 bp, Homo Sapiens
ccgttgggttttctcttccaggggttaattgtgaaattaattttgatgactgtgcaagtaacccttgtatccatggaatctgtatggatggcattaatcgctacagttgtgtctgctcaccaggattcacaggtaaagctccttttactgcaaggccc

SEQ ID NO: 719 - DNA, 135 bp, Homo Sapiens
ttctattcttccttgctttgtgcatgtttatctagactgctgatcttggacttgatattggtgcccagggagaaccccttggatatcgccaggatggtatgtgtctcatatttctcgattaactccagatcaagc

SEQ ID NO: 720 - DNA, 153 bp, Homo Sapiens
aggatggtcttgtgtctgtgttgactgattctcttgtagaccgagattttagatgaagataagcgcttaggcagtgcagtggattactactttattcaagatgacggaagcagatttaaggtaagcccctgactgcgacaagctaaaacccac

SEQ ID NO: 721 - DNA, 157 bp, Homo Sapiens
agttattttcaaacggtctggttttattttagtgctgttcctttgggaaccacggccaaagaagagatggagcggttctggaataagaatataggttcaaaccgtcctctgtctccccacattactatctacaggtaaggaaggattctggagccag

SEQ ID NO: 722 - DNA, 161 bp, Homo Sapiens
aaatggcccttgtcttgcaggtatgacataatgaagacttgctgggatgcagatcccctaaaaagaccaacattcaagcaaattgttcagctaattgagaagcagatttcagagagcaccaatcatgtgagtataccctggccaggcatagaatccccctt

SEQ ID NO: 723 - DNA, 170 bp, Homo Sapiens
tctcccttcctcctttgaacaaacagaacagaacaaacatgacctatgagaaaatgtccagagccctgcgccactactacaaactaaacattatcaggaaggagccaggacaaaggcttttgttcaggtagcacttcctttttctcctttccttcttttgggaggatgct

SEQ ID NO: 724 - DNA, 179 bp, Homo Sapiens
agttaaagttcggttgttttcgtatttcaggtcagcaaattaaaacgtcacattcgctctcatactggagagcgtccgtttcagtgcagtttgtgcagttatgccagcagggacacatacaagctgaaaaggcacatgagaacccattcaggtaggacttctccactccttactgtatt

SEQ ID NO: 725 - DNA, 154 bp, Homo Sapiens
gcaaggagccaggcatttttcttatctcaacatgtgtttgcagcctcctccaaaactgcccagtggagtgttcagtctggaatttcaagattttgtgaataaatggtaagttggctccttgttctctggaagcgtatactctggatttgtcagg

SEQ ID NO: 726 - DNA, 158 bp, Homo Sapiens
tccattggattccttcgcaagggtcaaagactctaaatggaggatctgatgctcaagatggtaatcagccacaacataacggggagagcaatgaagacagcaaagacaaccatgaagccagcacgaagaaaaagtgaggaataggttttaatgggtct

SEQ ID NO: 727 - DNA, 166 bp, Homo Sapiens
ttgtcagctttttgggggtcatttttattcaggatgaagatgcatcagggggcgatcaagatcaggaagaaagaagatggaacaaaaggactcagcagatgcttcatggtcttcaggtattgccgctgttgtctcagaggaaaatgcttcatcaaaggcttttgcc

SEQ ID NO: 728 - DNA, 150 bp, Homo Sapiens
ggtttacgtgcatattttctggcttacgggttttctttatttcctttcagatgcctggaagtcattgacagataaagtccaggaagctcgatcaaatgcccgcctaaagcagctctcatttgcaggtaatggctggaggtgttattccac

SEQ ID NO: 729 - DNA, 150 bp, Homo Sapiens
ggcactttccttctggttcagagcatgtgattgcgaccagcagattgatagctgtacgtatgaagcaatgtataatattcagtcccaggcgccatctatcaccgagagcagcacctttggtaagttgccattctgctactttaaaaatca

SEQ ID NO: 730 - DNA, 150 bp, Homo Sapiens
tggtggtgcatactttatttgtctagaatcgaacagatgtgcagtgccagcaccgatggcagaaagtactaaaccctgagctcatcaagggtccttggaccaaagaagaagatcagagagtaagttctttcttcattggtgtgtgactca

SEQ ID NO: 731 - DNA, 150 bp, Homo Sapiens
aagtggtttcctcagatagagcagtaattgtccatgctctctctaaccaggaaatgttaattttggaggccgtccacaacttccaggttcccatcctgcggtaagtgtcactaggaataccactgaatgagaagctgttgcatatttcac

SEQ ID NO: 732 - DNA, 150 bp, Homo Sapiens
agcgtttcagctccagactctttttgaccaatattgtttcctctttcagaagtgcctagctgccactccatttatatgaggcaagaaggcttcctggctcatcccagcagaacagaagttaagttgtccaccactgaaattccgtgtaaa

SEQ ID NO: 733 - DNA, 208 bp, Homo Sapiens
agcaaccatttttgtgcccatgttttctcattcccttatagggatcgtggaagaataccaattgccatattacaacatggtaccgagtgatccgtcatacgaagatatgcgtgaggttgtgtgtgtcaaacgtttgcggccaattgtgtctaatcggtggaacagtgatgaagtgagtggaactcagtcccctgaagaagtgattcgt

SEQ ID NO: 734 - DNA, 196 bp, Homo Sapiens
tatgtctcctcttccctgcccctgcagactatattgaactccatgcacaaataccagccccggttccacattgtaagagccaatgacatcttgaaactcccttatagtacatttcggacatacttgttccccgaaactgaattcatcgctgtgactgcataccagaatgataaggtaaactcaaggggctttcctt

SEQ ID NO: 735 - DNA, 198 bp, Homo Sapiens
gtcctgctcctgatcctgtttgtattgatttttctaaaaggcttttgtggccacaggaaccaatctgtctctccagttttttccggccagctggcagggagaacagcgacaaacacctagccgagagtatgtcgacttagaaagagaagcaggcaaggtaggaaacatttctttgcaatttaaaatactgtgggttgt

SEQ ID NO: 736 - DNA, 151 bp, Homo Sapiens
cttctccctggctctgactcacccttgttttataacagatgctgcaatgtacaacaactctgaagccctgcccacctctcctatggcacccacaacctatggtatatgtgattcctaattacacaaattaatttgaagggacttactgggg

SEQ ID NO: 737 - DNA, 151 bp, Homo Sapiens
atctcaatcgcctgctctccctttcttctttccagtatggtgactacgaccccagtgttcacaagcggggatttttggcccaagaggaattgcttccaaaaagggtaagagattaaattcccttttcaggaagacatagcagatatgtggt

SEQ ID NO: 738 - DNA, 182 bp, Homo Sapiens
agaccttttcctccctcattcacaggctggcttattagctgtaatggcccagatgggttgttacgtccctgctgaagtgtgcaggctcacaccaattgatagagtgtttactagacttggtgcctcagacagaataatgtcaggtgagttttttgtttcccacttaagttctcattcagtca

SEQ ID NO: 739 - DNA, 150 bp, Homo Sapiens
acatctgggattttgcttcattgtacatatgttttcttccttcagtgaccatgaaagacaccaggttgacagcactggaaactgaagtaccagttgtcgctagaacaggtaagctataaccaggccagtggttagaggaagatgggaggg

SEQ ID NO: 740 - DNA, 150 bp, Homo Sapiens
tcagaccacggtttctatttttcatctagaatgaagatgataaccgagccagtgagagcaagaaacccaaaacggaggacaagaattcagcaggccataagccatccagcaacagagagtacgttcccaaaacatgttgcatgccagtgt

SEQ ID NO: 741 - DNA, 150 bp, Homo Sapiens
atcttctgttacattctgcttcacaggccattctcgaatctccagaaaagcagctaacactaaatgagatctataactggttcacacgaatgtttgcttacttccgacgcaacgcggccacgtggaaggtaagctttctcaggcctgttt

SEQ ID NO: 742 - DNA, 193 bp, Homo Sapiens
ttttcaccccgctccccttagaactggaatgatgaatgggacaatcttatcaaaatggcttccacagacacacccatggcccgaagtggacttcagtacaactcactggaagaaatacacatatttgtcctttgcaacatcctcagaaggccaatcattgtcatttcaggtgagatgcctgcagatcacggat

SEQ ID NO: 743 - DNA, 153 bp, Homo Sapiens
tccttaaggcctctgtgctttttaacaaatggtttcttttgcagttggagttctctccacagacactgtgttgctacggcaaacagttgtgcacaatacctcgtgatgccacttattacagttaccagaacaggtaagcttggccaggtgtgg

SEQ ID NO: 744 - DNA, 198 bp, Homo Sapiens
ggtcatttgctgtgtttgttgacaggtgccaatttgctaattgacagcactggtcagagactaagaattgcagattttggagctgcagccaggttggcatcaaaaggaactggtgcaggagagtttcagggacaattactggggacaattgcatttatggcacctgaggtgagaagcatctttgagtgtgatgacaga

SEQ ID NO: 745 - DNA, 150 bp, Homo Sapiens
ccataatataaagttgttgcgttttgtatttcagaccattgccctcttgaacatttaccgtaaccctcaaaactcttcccagtctgctgacggtttgcgctgtaagttcatacaagttccttccccggttccctgggcttgcgtgtcaga

SEQ ID NO: 746 - DNA, 150 bp, Homo Sapiens
gttctgagttagctgcacatttacctgttggatgttatctgtatttgcagttgtgtactgtcagctatggaaaagtcaagctggtcttgaagcacaacaggtaagagattccatgacaggcctgtcccaaggcactgtcactttctgcgc

SEQ ID NO: 747 - DNA, 165 bp, Homo Sapiens
ttctggccttgttaatggcgtgttctgctttttctttcagtttccccctttctagggtgaggatggttctacacagccacccggagttccttagttgaaaggtgcgccctgctgtgacaggtattctttctttatgtttttctttcatgttaacaatcgaggggg

SEQ ID NO: 748 - DNA, 115 bp, Homo Sapiens
aaatcccttcctctctttctcagataacctgaggaccatggatgctgatgagggtcaagacatgtcccaagtttcaggtgagaccttatgagatagctgtgtgggaagttcatga

SEQ ID NO: 749 - DNA, 130 bp, Homo Sapiens
tacacccctgtcctctctgtcccagggaaattcaactactgaggaggttacggcacaaaaatgtcatccagctggtggatgtgttatacaacgaagagaagcagaaaatatatcctttccggtgttggga

SEQ ID NO: 750 - DNA, 242 bp, Homo Sapiens
agacctcagacaaggcatctcataggaggctttttcataaaactaggctctgctggtagtaaggaggccagtttggaggcaggcgttgagctgtgcacatctccccactccagccaccttctccatatccatcttttatttcatttttccacttggctgagccatccagaaccttttcaatgtataaaatggaatattcttacctcaattcctctgcctacgagtcctgtatggctgggatg

SEQ ID NO: 751 - DNA, 175 bp, Homo Sapiens
actttctctcctgccctcacaagggaaaaagaccttgatgaagttctgcagacccactcagtgtttgtaaatgtttcctaaggtcaggttgccaagaaggaagatctcatcagtgcgtttggaacagatgaccaaactgaaatctgtaagcaggcgggtaacagctgcagcatag

SEQ ID NO: 752 - DNA, 176 bp, Homo Sapiens
cacttcagttatgtacctgatgggtatttctaggcaagaggaggaacgacgtagaagagaggaagagatgatgattcgtcaacgtgagatggaagaacaaatgaggcgccaaagagaggaaagttacagccgaatgggctacatggatccagtaagtcagcctttactgccttcca

SEQ ID NO: 753 - DNA, 178 bp, Homo Sapiens
ttccccattccccattccaaggtaatgtaaatgggaaaaaaagaaaccacacaaagaggatacaggaccctacagaagatgctgaagctgaggacacacccaggaaaagactcaggacggacaagcacagtcttcggaaggtaattgtgttccaggtttgcttgacctgtcagagtgt

SEQ ID NO: 754 - DNA, 167 bp, Homo Sapiens
tgctgaacgcatttggcttatttctccctttcacactttcaaaaatagatcctcgtccctcccaagatgttgtttacacgaggggcttcataacggattctaacggaagacactgaaaaggtaacttgtcagagggggaagagggtggctgcttggggtagggttta

SEQ ID NO: 755 - DNA, 201 bp, Homo Sapiens
tcattgtgtcttccctcctctagctatcttaatgacttggaccgcgtagctgaccctgcctacctgcctacgcaacaagatgtgcttagagttcgagtccccaccacagggatcatcgaatacccctttgacttacaaagtgtcattttcaggtagtaactgagtccatgaaacctatttcccagcttttatgccttgagt

SEQ ID NO: 756 - DNA, 159 bp, Homo Sapiens
tgttgacaatgttttctcccacagggaaaaagcaagcatggttgtccctgaagaaagagaaggcagagatgaaacaaacttagacctagtaagaggcacagcatctgcagatgtttccactgacactcggaaagccggtgagtcctgcactttgtcagg

SEQ ID NO: 757 - DNA, 163 bp, Homo Sapiens
acatttttaatgctcctttctttgacagaaaaagcagacagctctgaaagagaggcactcatgtcagaactcaagatgatgacccagctgggaagccacgagaatattgtgaacctgctgggggcgtgcacactgtcaggtaacccacttccacgaaaatcac

SEQ ID NO: 758 - DNA, 202 bp, Homo Sapiens
agcaaaaggagtgacattcctaatgtgttctttctcccattcttctaggctgacaaacgggctcatcataatgcactggaacgaaaacgtagggaccacatcaaagacagctttcacagtttgcgggactcagtcccatcactccaaggagagaaggtgagtttcctgagaaagctgagtagctggccactactagcttgga

SEQ ID NO: 759 - DNA, 151 bp, Homo Sapiens
tctcatcatttcactgagatatgcatctattacttttacatttcaggccaaaagtgtgatccaagctgtcccaatgggagctgctggggtgcaggagaggagaactgccagaaacgtaagtcagtgaacagcctcagacccatgtgtgacc

SEQ ID NO: 760 - DNA, 195 bp, Homo Sapiens
tgaacttgtcacttcattggtcaatttaatgatttctacaggagcagtttttgcaagaaaggatcaaagtgaacggaaaagctgggaaccttggtggaggggtggtgaccatcgaaaggagcaagagcaagatcaccgtgacatccgaggtgcctttctccaaaaggtacaggagggaagtgtgtgtgtggcctg

SEQ ID NO: 761 - DNA, 196 bp, Homo Sapiens
aggcagcctttataaaagcaaattaacccatgtgggccttaatttttagacagcactaccacctggactggaagtaggactgcaccatacacacctaatttgcctcaccaccaaaacggccatcttcagcaccacccgcctatgccgccccatcccggacattactgtaagctcttgtttttgttgtaagggctat

SEQ ID NO: 762 - DNA, 150 bp, Homo Sapiens
acccattttccttcctggacatgctgcctgcagggccactcatggtgattgtggaattctgcaaatttggaaacctgtccacttacctgaggagcaagagaaatgaatttgtcccctacaaggtatgtcatctcctaatcctgctctggc

SEQ ID NO: 763 - DNA, 181 bp, Homo Sapiens
aaaaatttcccctgcgcttagattcttctactcaaaacaaaagagccaacagaacagaagaaaatgtttcagacggttccccaaatgccggttctgtggagcagacgcccaagaagcctggcctcagaagacgtcaaacgtaaacagctcggtgggttgatcactaaaggagcacgcactg

SEQ ID NO: 764 - DNA, 156 bp, Homo Sapiens
agacatgatgcttcgcttgaaggtaatctttacggttcttctctcaacagctttgaaaagtccagccgcatttcatgagcagagaaggagcttggagcgggccagggtaaggtaccttttttccccctcagaactcccaaagagcattttcagagc

SEQ ID NO: 765 - DNA, 184 bp, Homo Sapiens
tgcacgttgtttgtagctgtagtgcttgattttgggtttctttcacagataaacttctgcactggaggggcctctcctcccctcgttctgcacatggagctctaatccccacgcctgggatgagtgcagaatatgccccgcagggtatttgtaagttgagccttatttcttctacaaatgtcca

SEQ ID NO: 766 - DNA, 175 bp, Homo Sapiens
gtttcccctggatttatgtggtagtagttaactgctgcttctgtttttaggtttcagaagcaggcaacaggaacaagatgtgaactgtttctcttctgcagaaaaagaggctcttcctcctcctcccgcgacggtgggtgtgctgtcctttatcgctgcagtaaaggcgaaggtg

SEQ ID NO: 767 - DNA, 150 bp, Homo Sapiens
acgtgcatgtcctttttcccttttcgtgttctgcaggtggacgttgccataagtcctgtactggccgttgctggggacccacagaaaatcattgccagacttgtaagtgttcatcagtgagagcacacaggtttgatgtggtcaaggaat

SEQ ID NO: 768 - DNA, 150 bp, Homo Sapiens
tgtctctacctcctacatcttatctccaggttggatgattgatgagaacattcgcccaacctttaaagaactagccaatgagttcaccaggatggcccgagacccaccacggtatctggtcataaaggtgagtagggagtaggaggtgct

SEQ ID NO: 769 - DNA, 152 bp, Homo Sapiens
ggttgtttctatttgctaatgctgtttctgttgacttttgacttttctagtttcccagagctatggggacttcccatccggcgttcctggtcttaggctgtcttctcacaggtacggagcccagtcctctctgagttccttgtttgggtgtc

SEQ ID NO: 770 - DNA, 164 bp, Homo Sapiens
agcacttcctgaaataatttcaccttcgtttttttccttctgcaggaggacaccatggaggtggaagagttcttgaaagaagctgcagtcatgaaagagatcaaacaccctaacctggtgcagctccttggtgagtaagcccggggctctgaagagagggtctc

SEQ ID NO: 771 - DNA, 194 bp, Homo Sapiens
atgggcctcactgtctgtttttgctataggtgggaactgcaagatacatggctccagaagtcctagaatccaggatgaatttggagaatgttgagtccttcaagcagaccgatgtctactccatggctctggtgctctgggaaatgacatctcgctgtaatgcagtgggaggtaggtgtggaccagcatcattg

SEQ ID NO: 772 - DNA, 151 bp, Homo Sapiens
gtcctcatggctctgtgactgtgcctcttgtcaggtgtatgagtttagagtcaaagaatctagcatcatagctccagctcccgctgaggatgtggatactcctccaaggaaaaagaagaggaaacaccggtgagtctgtcatgaagctcct

SEQ ID NO: 773 - DNA, 173 bp, Homo Sapiens
cccacctgcattgttcatcatgttaatgccagttcttttttaggtatcatctttatcagaaagtgaggagtcccaggactcatccgacagcataggctcctcacagaaagcccacgggatcctagcacggcgcccatcttacaggtgagtactctcttgtatgaagccctgca

SEQ ID NO: 774 - DNA, 152 bp, Homo Sapiens
ggataactatgttcttccttttcatcatagatattcttactgattctccgggctctgcagctcttgacccggctggtattgtctctacagccaattcctctgaagtcagcaacagcaaaggtgaggtgcgcagggctgccagagaagaggag

SEQ ID NO: 775 - DNA, 154 bp, Homo Sapiens
ctggcacacttcttcacctcctctccttactcttgtttccagatcctgcccctgagctttcatgagctgttgaaccatctggaattcacaggcctgtcatgagagacacgatgagaagtccttaaaggtagatcactgattcacaggggagcag

SEQ ID NO: 776 - DNA, 156 bp, Homo Sapiens
cctcagggatggtagtgacagtcttatttcctattgatacaggatttttttccttgattccttcgtgggactcaagacaggggtgccctgtttactcagcccactgtgctcaacctcttgcaggagtgtgcaggtgagtgagcaagtgcaggatct

SEQ ID NO: 777 - DNA, 158 bp, Homo Sapiens
catccatggaatatgttcttttgcatacagatgccatctcatccggagatgatgaggatgacaccgatggtgcggaagattttgtcagtgagaacagtaacaacaagagtaagtaactgcccggctccgatggtccccgagagaggagcatggaggga

SEQ ID NO: 778 - DNA, 182 bp, Homo Sapiens
ccttgcactcttgtggttgtttttcccattacaggtagagttggctttgtgggacacagctgggcaggaagattatgatcgcctgaggcccctctcctacccagataccgatgttatactgatgtgtttttccatcgacagccctgatagtttaggtgagtggccctgcaccctgatatttg

SEQ ID NO: 779 - DNA, 157 bp, Homo Sapiens
aaggtttccaattcacctttcagctcttcatggaaccagccaggagagagaactggtcagctgctaatcaccaaatgaactccctgatctggcctagggaacagtgggattcacaggcatgggtgacttagaaaaccgggcccagcagaaatgaatc

SEQ ID NO: 780 - DNA, 169 bp, Homo Sapiens
ggtccccatccattcttcctattccctttaggttgttacactctggtaccgagctcccgaagttcttctgcagtccacatatgcaacacctgtggacatgtggagtgttggctgtatctttgcagagatgtttcgtcgaaagtatgggacccacataccctggactacc

SEQ ID NO: 781 - DNA, 150 bp, Homo Sapiens
ccaatctgcttatgaccaggagccactcaagcagcactctcccttcacaggtggtattccaaacacatgactcggagtcaggctgagcaactgctaaagcaagaggtaagtgtggaaccactagcacacagcattctccttgcataagtg

SEQ ID NO: 782 - DNA, 150 bp, Homo Sapiens
ggtgggattttgttgtttgcagctttgactctcccggatcttgcagagcagtttgcccctcctgacattgccccgcctcttcttatcaagctcgtggaagccattgaaaagaaaggtaaccagactgctagagggcatcagttcctttgt

SEQ ID NO: 783 - DNA, 150 bp, Homo Sapiens
tgaccaatttggcttcgtcctcttcctttgcagaaagctgcaggagacacagatgtccaccacctcaaagctggaggaagctgagcataaggttcagagcctacaaacaggtttgatactctccttcctagtaccatggatgtggggagg

SEQ ID NO: 784 - DNA, 150 bp, Homo Sapiens
tcaaagctgcttctgtcatctgtgtgaacatgcgcttttctctctgcagaacctgagagccagaagcaacaaagatgccaaggatccaacgaccaagaactctctggaaagtgagttctgcatgctgaggtctctgtgtgccctcgtcag

SEQ ID NO: 785 - DNA, 150 bp, Homo Sapiens
ggctaatggttctcagagctaagtatcaaggatttcattctcctttgtagggatcctggagcgggttgtgagaaggaatgggcgcgtggatcgtagcctgaaagacgagtgtgatacggtgaaaggatggaggctgtgcaatgggagaat

SEQ ID NO: 786 - DNA, 151 bp, Homo Sapiens
gcctttcaattcactgtcctcactctgacttctcttgtttgttctagaactttgctccccagctgtcttatggctatgatgagaaatcaaccggaggaatttccgtgcctggccccatggtgagccagcagggggagcatggatgacagaa

SEQ ID NO: 787 - DNA, 190 bp, Homo Sapiens
tcagcagggtttttcttgcttgttttcaggctttgtggatttgaccctccatgatcaggtccaccttctagaatgtgcctggctagagatcctgatgattggtctcgtctggcgctccatggagcacccagggaagctactgtttgctcctaacttgctcttggacaggtaagtgacctggctgtagctt

SEQ ID NO: 788 - DNA, 151 bp, Homo Sapiens
ggttttcctctccttccccacagggcgagctactatagaaagggaggctgtgccatgctgccagttaagtggatgcccccagaggccttcatggaaggaatattcacttctaaaacagacacatggtaagtcagccatcatcctccaggta

SEQ ID NO: 789 - DNA, 138 bp, Homo Sapiens
ccatgacactccttccacctcatggcccctttctgttttccagcaagatctttgcaggagtttgccactgtcctcaggaatcttgaagatgaacggatacggatggtgagtagggctgggctactcttggtcccagat

SEQ ID NO: 790 - DNA, 153 bp, Homo Sapiens
tgtaattcctggcttctaggtttctaattctgattttctcctccagaaagatccccagcaggccctcaaggagctggctaagatgtgtatcctggccgactgcacattgatcctcgcctggaggtgagatgagggcttccctgcctcattcag

SEQ ID NO: 791 - DNA, 151 bp, Homo Sapiens
cgtgttcccgtttcctcttgatctcccaggtatttctttgctgtgctggcgatcctcaccatcctcggcgttctcaatgggctggttttgcttcccgtgcttttgtctttctttggaccatatcctgaggtcagtagtgacacggggatgt

SEQ ID NO: 792 - DNA, 178 bp, Homo Sapiens
cctctgtgtatctccttcccaggtaccgcatgcacaagtcccggatgtacagccagtgtgtccgaatgaggcacctctctcaagagtttggatggctccaaatcaccccccaggaattcctgtgcatgaaagcactgctactcttcagcattagtaagtgcctagaagtgcagggaat

SEQ ID NO: 793 - DNA, 172 bp, Homo Sapiens
gctttctttttgctcccccagggcctggtgaaatccccatgggaatgggggctaatccctatggccaagcagcagcatctaaccaactgggttcctggcccgatggcatgttgtccatggaacaagtttctcatggcactcaaaataggtggggtgttattttgtgactctg

SEQ ID NO: 794 - DNA, 150 bp, Homo Sapiens
acttttaccctggatttgcccattcagaacagccaccccatcttttctctcaactgggagtgtgtggtcagtttcctgtggaacacagaggctgcctgtcccattcagacaacgacggatacagaccaggtacgtgtgctttcacctggc

SEQ ID NO: 795 - DNA, 157 bp, Homo Sapiens
ggtggggttttgttaacgtgaatttaatctttttgacagaaataacagcaggctggggaatggagtgctgtatgcctctgtgaacccggagtacttcagcgctgctgatggtaagagtccgggccaccagcactgccagcgtgcagggcaggtagat

SEQ ID NO: 796 - DNA, 157 bp, Homo Sapiens
agtgtaaagttaaccttgctgtgtattttcccttattttaggctgctcctgcgtttggtggatgatttcttgttggtgacacctcacctcacccacgcgaaaaccttcctcaggtgaggcccgtgccgtgtgtctgtggggacctccacagcctgtg

SEQ ID NO: 797 - DNA, 153 bp, Homo Sapiens
gtatcaaggctgccctgactgtcatgctccctgtcttccacagcgggagtcgtgtgaggttggctgtagcagcgcggaaggtgcatatgaagaggaagtactgggtaagaggacacacacgacttttaaaaaataggctgcaccactctagtg

SEQ ID NO: 798 - DNA, 181 bp, Homo Sapiens
ttcaggccaccaacctcattctgttttgttctctatcgtgtccccacagggaaaagcttcactctgaccatcactgtcttcacaaacccaccgcaagtcgccacctaccacagagccatcaaaatcacagtggatgggccccgagaacctcgaagtaagtgcatccacttggggctggtac

SEQ ID NO: 799 - DNA, 156 bp, Homo Sapiens
gtgctgattccctgatgtgccttctacctcttttcttctctcccgccagggagctcgaggacaatatgagtgaccgggttcagtttgtgatcacagcacaggaatgggatcccagctttgaggaggtgagtaccaaagaggcagagaatgggagac

SEQ ID NO: 800 - DNA, 156 bp, Homo Sapiens
accaacatggatggagtggtcactgtgacgcccagaagtatggacgcagaaacctacgtggaaggccagcgcatctcagaaaccaccatgctgcagagtggcatgaaagtgcagtttggggcgtcccatgtatttaagtttgtggaccccagtcag

SEQ ID NO: 801 - DNA, 156 bp, Homo Sapiens
tgcccaccctaatcctgtgtttctttgcctcctatagacatgattcctatggcaatcagttctccacccaaggcaccccttctggcagccccttccccagccagcagactacaatgtatcaacagcaacagcaggtgaggagggtagctgggaatg

SEQ ID NO: 802 - DNA, 154 bp, Homo Sapiens
ctgcctctcttttctccccatacaggacgggctctacgagtgcattctctgtgcctgctgtagcaccagctgccccagctactggtggaacggagacaaatatctggggcctgcagttcttatgcaggtgaggtgctccttaattgctttaaga

SEQ ID NO: 803 - DNA, 184 bp, Homo Sapiens
gaagtcatgggctgcttgtcctgtgctctctccccaggagaaaagccttacagatgctcatgggaagggtgtgagtggcgttttgcaagaagtgatgagttaaccaggcacttccgaaagcacaccggggccaagccttttaaatgctcccactgtgacaggtacgtgcctgaggacaatgctg

SEQ ID NO: 804 - DNA, 150 bp, Homo Sapiens
ccaagctgtgaaggccttttaacagaccaccttccttctgattcccagagaccccaccccctggctacctgagtgaagatggagaaaccagtgaccaccagatgaaccacagcatggacgcaggtcagtcatgcagggtcatgctcttat

SEQ ID NO: 805 - DNA, 150 bp, Homo Sapiens
cctccttctcacgtgtctgtgtttcttttctcctccatgctatggcagtggtgccccgtgctgatgagacaatacttggtgcagccccaggcagtccttttccaggtaatttcctagggacccaaatgatgcccagtgcacactctcctg

SEQ ID NO: 806 - DNA, 204 bp, Homo Sapiens
agcagagtgacccagtgatgtttgtctgttacagatcacggatacacgactctagccaccagtgtgaccctgttaaaagcctcggaagtggaagagattctggatggcaacgatgagaagtacaaggctgtgtccatcagcacagagccccccacctacctcaggtaatgcgttcctggccagggcatctctggggacacctgt

SEQ ID NO: 807 - DNA, 130 bp, Homo Sapiens
tcctctctggatcctcgtgaggtataaagacgagtcctccaccaccagtcaggcacactctaccaccatgaatccactcctgatccttacctttgtggcagctgctcgtgagtatcatgccctgcctcag

SEQ ID NO: 808 - DNA, 150 bp, Homo Sapiens
ggagctgctcctcatcctactcacctttccctcatagtccggaagaccaagggcaaccgaagtacctcacctgtcactgaccccagcatccccattaggaagaaatcaaaggatggcaaaggtatggacagctgggactcaggtgaggtg

SEQ ID NO: 809 - DNA, 150 bp, Homo Sapiens
tgaagtttttgtctgtttctccccctgcagcatctgatgctgttcagatgcagagagagtggagctttgcgcggacacaccctctgctcacctcactgtaccgcagggtgagtggatgtggtattatacctgcttctgagctcgtggcgg

SEQ ID NO: 810 - DNA, 157 bp, Homo Sapiens
actggatctgcttcacacctaggtcccgacatctgtggccctggcaccaagaaggttcatgtcatcttcaactacaagggcaagaacgtgctgatcaacaaggacatccgttgcaaggtgtgcctgggggtggtggcaaatggctgtcatggggaga

SEQ ID NO: 811 - DNA, 157 bp, Homo Sapiens
ctttccctcattccctccccactgcctacttctacttcctcccaggttatgagcagccgttttctgccctacgacaacatcatcacagacgccgtgctcagccttgacgaggacacggtgctttcaacaacagaggtaagaacccatgcctgaggag

SEQ ID NO: 812 - DNA, 151 bp, Homo Sapiens
ttgggcctgtgttatctcctaggttggctctgactgtaccaccatccactacaactacatgtgtaacagttcctgcatgggcggcatgaaccggaggcccatcctcaccatcatcacactggaagactccaggtcaggagccacttgccac

SEQ ID NO: 813 - DNA, 151 bp, Homo Sapiens
cctgacccttctccctatccccagctatgagatcatgcagaagtgctgggaagagaagtttgagattcggccccccttctcccagctggtgctgcttctcgagagactgttgggcgaaggttacaaaaaggtatgttgaggcagggtaggg

SEQ ID NO: 814 - DNA, 185 bp, Homo Sapiens
tccctttctcccctctttgaatgaagagggaaggtgcccacctgtccaccatggctgtgaagatgatccgtgccctgagggatccgaatgtgtgtctgatccctgggaggagaaacacacctgtgtctgtcccagcggcaggtttggtcagtgcccaggtgagagttgaatggttgggtatactt

SEQ ID NO: 815 - DNA, 152 bp, Homo Sapiens
actcaagtccctttcccctctctaccctctcaggactggcagccaccctttgctgtggaagtggacaacttcaggtttaccccccgaatccagaggctgaatgagctagaggtgagaagactaggagctggtggtggggttgggagaatgta

SEQ ID NO: 816 - DNA, 169 bp, Homo Sapiens
tcttcacttcagttgcccctacctattcttgctctcctccgcaggtccagggcttactggagaatggagacagtgtgaccagtcctgagaaggtagccccggaggagggctcaggtaagagaggtaggtctaggtgtggtgtgggtaggctgttgactgcacatcacca

SEQ ID NO: 817 - DNA, 152 bp, Homo Sapiens
ccacactgagcctttttcccttctttgtgtttgcagccacagcacagggtacgagagcgataaccacacaacgcccatcctctgcggagcccaatacagaatacacacgcacggtgtcttcagaggcattcaggtgagcacacagacaggcc

SEQ ID NO: 818 - DNA, 150 bp, Homo Sapiens
catgatgcgctgtgtgtccctgcttctagatgccgacaaaaggatcaaggtggcgaagcccgtggtggagatggatggtgatgagatgacccgtattatctggcagttcatcaaggagaaggtagtgccccctcctgaagtgggtggctc

SEQ ID NO: 819 - DNA, 137 bp, Homo Sapiens
aggaagggcagtgaggattcactggagtctcttcacctctcccaggcatgtcagccacgtggggtgggacccccagaatggatttgacgtgagtaacttcagagtctcttggactccactaaacttccacccaccct

SEQ ID NO: 820 - DNA, 115 bp, Homo Sapiens
caaaaagtgccagccctcacctccctgtcttcttgtctaggttttccgtgtgtatcagggccaacagccagggacctgtatggtaagtctcctaggcctctcccaaccgtgtctc

SEQ ID NO: 821 - DNA, 184 bp, Homo Sapiens
actaagttgccacaggacctgcagcctgcccactctcccctaggtgccgccggatggtggtggttgtctctgatgattacctgcagagcaaggaatgtgacttccagaccaaatttgcactcagcctctctccaggtaagctcaaccctgctctggcaagagaatgagggaatgtgtaggtggg

SEQ ID NO: 822 - DNA, 153 bp, Homo Sapiens
gggctctggggcattaacatatcccattgtgtcctgtttccaggagcctgactacggggccctgtatgagggacgcaaccctggcttctatgtagaggcaaaccctatgccaactttcaaggtacagctcaggcctctgggcataggaagctg

SEQ ID NO: 823 - DNA, 142 bp, Homo Sapiens
cgaagtctcgctcttttcccataggctggagtgcaatggtgtgatctcagctcactgcaacctctgcttcctgggtttaagtgattctcctgcctcagcctcccgagtagctgggattacaggtgactgccaccacgctcag

SEQ ID NO: 824 - DNA, 156 bp, Homo Sapiens
tgttttgttgttcttggcattttctaggagaagcaacagtttcagcgccatctgacccgcccaccaccccagtaccaagacccgacacaaggcagcttcccacagcaggttggacagttcacaggtagggggtgtctgtgtgacgagcagggacag

SEQ ID NO: 825 - DNA, 174 bp, Homo Sapiens
acctgcccagatccttaacctcagcctcttctccagcaggctggagagcacagaagcagagatgcatattccctcagccctagagcctagcacgtccagctccccaaggggcagcacagattcccttaaccaaggtgggtaaaccaatagctaggccattgtcttctgggtacg

SEQ ID NO: 826 - DNA, 119 bp, Homo Sapiens
ctctctccacccaaacccttgtaggatggcagctgtgacccgggatttcggtgagatgcttctgcactctggccgggtcctgccagccgaaggtaagttttcagttccatttcaaagcc

SEQ ID NO: 827 - DNA, 162 bp, Homo Sapiens
tcccctcctcttcttgttctctcattagcttcgcctcaacagcatcaagaagctgtccaccatcgccttggcccttggggttgaaaggacccgaagtgagcttctgcctttccttacaggtaacaaaggggacccctggggcccagatgtggggactcttgg

SEQ ID NO: 828 - DNA, 150 bp, Homo Sapiens
cgacttcagtcttccacttcctatttccacccagttccagcgccaggggcttcagcagacccagcagcagcaacagacagcagctttggtccggcaacttcaacaacagctctctagtaagcctgcctgccttcccaaggagaaccccat

SEQ ID NO: 829 - DNA, 150 bp, Homo Sapiens
tcagcaggaagtgttgaccttttggcctttgtctccttgcaggcaggtgacagcagggacatgtctcgggagatgcaggatgtagacctcgctgaggtgaagcctttggtggagaaaggggaggtgagtggagatcttcctggctacccc

SEQ ID NO: 830 - DNA, 150 bp, Homo Sapiens
gaggccaggcatttttcactagggcctctgctttgcagacagatcttggagctgccctggaaggaggaaactttcttggtgttgcagtcactcctagagcggcaggtgagcaggctgccctggggaagagtggacaaagaagtgctgcag

SEQ ID NO: 831 - DNA, 157 bp, Homo Sapiens
caacacagtctctccctccagcatctggttttgtagcctggatgtgtctgctagtagccgaatggtggtcacaggagacaacgtggggaacgtgatcctgctgaacatggacggcaaagaggtgcgttctccgaggtcctgcctttccctccctcac

SEQ ID NO: 832 - DNA, 155 bp, Homo Sapiens
ttgctgctgcctcgcttatcgtgacctctgttgctctccagatcatcggccgtggcaatgaccaggtggccatcagctccaaatttgagacccgggaggatattggtatgctgccagtggggctggtttctgtgggttccaagaggggaggtgta

SEQ ID NO: 833 - DNA, 162 bp, Homo Sapiens
actgttctgacacaccccacccctctctgcaggtggagtgaccacctttgtggccctctatgactatgagtctaggacggagacagacctgtccttcaagaaaggcgagcggctccagattgtcaacaacacgtgagtgcccccttccctattgcccctcag

SEQ ID NO: 834 - DNA, 169 bp, Homo Sapiens
ctctaaatccctcgccctggctgtgtcctcaggtgctgtgtggccagtcagcagagggacaggaatcattcggccactgttcagacgggagccacacccttctccaatccaagcctggctccagaagagtgagtgtctttacctgacattactgagatctgccgagtgc

SEQ ID NO: 835 - DNA, 156 bp, Homo Sapiens
aacctctacccacccattcctttccagggcactgaagccaaaggcagaagttgatgaggatggagttgtgatgtgctcaggccctgaggagggagaggaggtgggccaggtgaaagggctggggcaagaatggtctggaggtgatggaagggatga

SEQ ID NO: 836 - DNA, 179 bp, Homo Sapiens
tcctctctccccactctcagtctgcagccaggagagcagggacgtcctgtgcgaactgtcagaccaccacaaccacactctggaggaggaatgccaatggggaccctgtctgcaatgcctgtgggctctactacaagcttcacaatgtaagtggactgggatcagcaagaacagggctc

SEQ ID NO: 837 - DNA, 138 bp, Homo Sapiens
aaaaacgggtggttgggcgccgctgtcttttcagtcgggcgctgagtggtttttcggatcatgtctggtggctccgcggattataacaggtatgcagtctgttggcggtcgcggtctgtagtgaaggtcatagggcgc

SEQ ID NO: 838 - DNA, 150 bp, Homo Sapiens
acagacagccgaacagacacggcaggtctcatgagccttcccagccaccgtagtgccggtgccctgagaacaggactgagtgatggcttccaactccagcgatggtgaggctgagtcctgttactatagcaacttcctaggcacactctg

SEQ ID NO: 839 - DNA, 156 bp, Homo Sapiens
ttctttttagtctagtgctccactagctcctctcctactgagctggggtaagaagcggagcgtatacggaggaggcgggatgcatttctgcatcgagcgcacaaaggtgtggcggagggggctccagagctgggaggggtcaatctacgggcgaat

SEQ ID NO: 840 - DNA, 154 bp, Homo Sapiens
taagcccgggacttccttgcctctcttggtagtggtgaatctggagctggcaagacggagaacaccaagaaggtcatccagtatctggcgtacgtggcgtcctcgcacaagagcaagaaggaccaggtgagtgctgcagcccttgtccctgtgg

SEQ ID NO: 841 - DNA, 150 bp, Homo Sapiens
ggggttttgattggctgagggtggagtttgtatctgcaggtttagcgccactctgctggctgaggctgcggagagtgtgcggctccaggtgggctcacgcggtgagtcatatggggaacttctgttgggtgtttggtggttcgaatccca

SEQ ID NO: 842 - DNA, 150 bp, Homo Sapiens
cctagggtgaggcttatgggcttttactcctcagggcaggagacgctgcagagcattacttggacctgctggccctgttgctggatagctcggagccaagggtgggtgtgtcttcaagcttctctgcaatggggtagacgggttggtgtc

SEQ ID NO: 843 - DNA, 151 bp, Homo Sapiens
gggtggccattaacacacaatgggctttctatcctgggcctcagatcgttggtgtctgcactgaagagctacactcagcccagcagtggaacgggcagggcatcctggagctgctgcggacagtgcctatgtgagtacccatgcaaggtgg

SEQ ID NO: 844 - DNA, 334 bp, Homo Sapiens
ctgcgaggaggggagaattcttggggctgagctgggagcccggcaactctagtatttaggataaccttgtgccttggaaatgcaaactcaccgctccaatgcctactgagtagggggagcaaatcgtgccttgtcattttatttggaggtttcctgcctccttcccgaggctacagcagacccccatgagagaaggaggggagcaggcccgtggcaggaggagggctcagggagctgagatcccgacaagcccgccagccccagccgctcctccacgcctgtccttagaaaggggtggaaacatagggacttggggcttggaacctaaggttgt

SEQ ID NO: 845 - DNA, 150 bp, Homo Sapiens
tcccattcccgtgtttccttgcagtgacagcccacacagcgagccaggggccatcgatgaagttgaccatgacaatggcactgagcctcataccagcgatgaaggtgagtgagggggatcctggggacaagggattcttcctggggcctc

SEQ ID NO: 846 - DNA, 129 bp, Homo Sapiens
aggctctgatgtgcttctctctctccctttgcagccgctgtacaaccagccctccgacacccggcagtatcatgagaacatcaaaatgtgagtgctcgcgggcagccgtgcagacacacagaggcaggg

SEQ ID NO: 847 - DNA, 165 bp, Homo Sapiens
gttttctgtctgcctctgccattcccagtgtgaccactcgtgctcagccgtatctcagcaggaggacaggtgccggagcagctcgtgcagctaagcagccaactgcagaaacgtcaggtgggtggtgcattcgcaggcatgctgaagaagcagttccaggcatgg

SEQ ID NO: 848 - DNA, 150 bp, Homo Sapiens
accctcaccctaaatctggcacctgcttctccatctccagagcacaagacgtcccccacccaatgcccggcagctggagaggtctccaacaagcttccaaaatggcctggtgagtgatgcgggatctctctgccctgggtggtggagatg

SEQ ID NO: 849 - DNA, 150 bp, Homo Sapiens
ctgagcctgccctactctgtatctccccgtatagatccgtggccagctgcagtcgcacggcgtgcaagcacgggaggttcggctgatgcggaacaaatcttcaggtgagcttttgttctagtgccctccccttcaagtggccagcctcag

SEQ ID NO: 850 - DNA, 150 bp, Homo Sapiens
cgcgcgtacacacacacacacacacacacacacacacacacacacacacacacgttcttatgtaaccgagcccgggtaaagcagggctgcagaaagcagaaacggcgagcccggctcctgggagcaggtgggacctcctttggcctttgg

SEQ ID NO: 851 - DNA, 199 bp, Homo Sapiens
cctctctccttctgcctcagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccggtaagtgagggtgatgtcccaggcagcctt

SEQ ID NO: 852 - DNA, 153 bp, Homo Sapiens
tcgctgttagacatctctctcactgcctgtctctggttctgtcctcaggccacccctgttctccgatgtgtaagggctcccgctgctggggagagagttctgaggattgtcagagccgtgagtctcagggaggcctggagtcagggaagggga

SEQ ID NO: 853 - DNA, 207 bp, Homo Sapiens
tgagaccccttcagaccctacagagaccccactgctctcacagctacaactactgccgggaagacgaggagatctacaaggagttctttgaagtagccaatgatgtcatccccaacctgctgaaggaggcagccagcttgctggaggcgggcgaggagcggccgggggagcaaagccaggtgaaaggctggagctccagcctgtgtc

SEQ ID NO: 854 - DNA, 154 bp, Homo Sapiens
gtgtttccttggggtcatgggggtggcttcatgttagtttttgcaggatccagatgaagaaatggccaaaatcgacaggacggcgagggaccagtgtgggagccaggtaggtccgcccggggttgggcctctgtggaggtccttctccccctgg

SEQ ID NO: 855 - DNA, 118 bp, Homo Sapiens
ctcgctcatccccgaggggcccctgcaacctctccgcgcgaagacggcttcagccctgcagggaaagaaaagtaacttcgcttttctcggaggaaccaggaaggattaagcggcttgg

SEQ ID NO: 856 - DNA, 160 bp, Homo Sapiens
ggcagctccgggtctataaagagaggcgtccgaggacgcgcagggagatttggacgctccggcctgggaggtgcgtcagatccgagctcgccatccagtttcctctccactagtccccccagttggagatctgtaagtagtagttgtcattctgggggca

SEQ ID NO: 857 - DNA, 175 bp, Homo Sapiens
ccctcaccttcccctcttttcccagagctgtcttcccagcccaccatccccatcgtgggcatcattgctggcctggttctccttggagctgtgatcactggagctgtggtcgctgccgtgatgtggaggaggaagagctcaggtggagaaggggtgaagggtggggtctgagatt

SEQ ID NO: 858 - DNA, 154 bp, Homo Sapiens
ctgtccttccctgacctcagcccaacctctactgtgtgcctctgcaggctcggatggcgggtgagcgaggagccagtgctgtcctctttgacatcactgaggatcgagctgctgctgagcaggtacccagggacatttgcgtgttcaaggtggg

SEQ ID NO: 859 - DNA, 185 bp, Homo Sapiens
tggtctctcggcgggaagccgtgcacgcctccagcgttgacactttcccggtgcactttttctggtgggaggggagagcggagcaggctcacgtgtaaccgcgcaggagcctcctctggcttgagccctttcttggtaagtcccaaaccttcccaagacaaccttggccttagcagtagaggagg

SEQ ID NO: 860 - DNA, 150 bp, Homo Sapiens
tgagttaacggctgcctctttctcctggacagggatgggatccccccatacaggatccgtaagcagcaccgcagggagatgcaggagagcgtgcaggtcaatgggcgggtgcccctacctcacattcccgtaagtaccggctttgcggtc

SEQ ID NO: 861 - DNA, 155 bp, Homo Sapiens
cacctggctccactgtgtagctgaggacctgtggctgagcccgctgaccatggaagatcttgtctgctacagcttccaggtggccagagggatggagttcctggcttcccgaaaggtgagcttcccccgaaggcccttcagacgggaaaagggtg

SEQ ID NO: 862 - DNA, 161 bp, Homo Sapiens
cccctgctaatgtctgaggtcccccttctgttcaggagtcatgactctgttctccatcaagagcaaccaccccgggctgctgagtgagaaggctgccagcaagatcaacgagaccatgctgcgcctgggtgagtggccccgggggacttcggtctgaggtc

SEQ ID NO: 863 - DNA, 150 bp, Homo Sapiens
ctggccacactgggtctccctaacacactcctcttctcacccctgcagcccccgcagcctgatgacctctccattgtgtgtttcacaagcggcacgacaggtaagcagaggcacgcagatccccagccatggctacctgcacccttccct

SEQ ID NO: 864 - DNA, 163 bp, Homo Sapiens
cactgtggccttgtttcctgcctgcaggcttggcgggggctccgaggacgccaaggagatcatgcagcatcgcttctttgccggtatcgtgtggcagcacgtgtacgagaagaaggtgcggctgctccccgcatattcacgcgcacgcatgctccccacatat

SEQ ID NO: 865 - DNA, 199 bp, Homo Sapiens
ctcactcctcccctgctcgctgcaggcccgggaccgtggcgcttcgagagattcgtcgttatcagaagtcgaccgagctgctcatccggaagctgcccttccagaggttggtgagggagatcgcgcaggatttcaaaaccgacctgaggtttcagagcgcagccatcggtgcgctgcaggtaagacaaaggcctggagc

SEQ ID NO: 866 - DNA, 197 bp, Homo Sapiens
tagttggcgagtgggctttaggacccaacgggaacccgtgcctcttgcagcagcctaacccagaagcaggggggaatcctgaatcgagctgagagggcttccccggttctcctgggaaccccatcggccccctgccagcacacacctgagcaggtaggaccatgcacaccccttcccaattctttggccgcctttga

SEQ ID NO: 867 - DNA, 170 bp, Homo Sapiens
ctggtttagcgacacgagcaccgcttcttcctcagtaccgcgccggagccttccgcagctgccgcttcagtccgaaggaggaagggaaccaacccactttctcggcgccgcggctcttttctaaaagtgtgagtggccccgggagagggaattgagggggagaaagtggg

SEQ ID NO: 868 - DNA, 150 bp, Homo Sapiens
cctctgctgactctgtctccccaggcagctgcattcagcctcagcggaggacacgcctgtggtgcagttggcagccgagaccccaacagcagagagcaaggtaaggggtgcttgtgtgggtacctgtgctcctggcctggtcgtgtagaa

SEQ ID NO: 869 - DNA, 120 bp, Homo Sapiens
cattggttgcggccatctctgccttgcagacgctccatcctcgggagatgacgaagacggggaggacgaggctgaggacacaggtgtggacacaggtaggagcagggtccagggttcagg

SEQ ID NO: 870 - DNA, 150 bp, Homo Sapiens
ccactgcaacccgactccggagctccgagcatcccttagttttaagtcatggcgggtgcgaacgggtctctgctgcaggcggctccgtgacagctcctgcttcacatgggtagaggagagacggcaaacgtcggggctcccaggacttcg

SEQ ID NO: 871 - DNA, 250 bp, Homo Sapiens
caggagggcggggtaaagccgctttcctctcctttctccctcccccttgtctgcgccacagcccccttctctccccgccccccgggtgtgtcagatttttcagttaataatatcccccgagcttcaaagcgcaggctgtgacagtcatctgtctggacgcgctgggtggatgcggggggctcctgggaactgtgttggagccgagcaagcgctagccaggcgcaagcgcgcacagactgtagccatccga

SEQ ID NO: 872 - DNA, 158 bp, Homo Sapiens
cgcctcttcccaccctagacctggacaaggaggatggacggcccctggagctccgggacctgcttcacttctccagccaagtagcccagggcatggccttcctcgcttccaagaatgtgagtaggaacctggccctggctcatagccacccaggtctg

SEQ ID NO: 873 - DNA, 119 bp, Homo Sapiens
cgtgaccgacatgtggctgtattggtgcagcccgccagggtgtcactggagacagaatggaggtgctgccggactcggaaatggggtaggtgctggagccaccatggccaggcttgctg

SEQ ID NO: 874 - DNA, 151 bp, Homo Sapiens
ctgagacctggggactgatcctcctgcacccctccccagcaccatcgtgaagagtggtctccgtttcgtggcgccagatgccttccatttcactcctcggctcagtcgcctgtgagtgtggccagtgctgggcagtgggagttggggagga

SEQ ID NO: 875 - DNA, 157 bp, Homo Sapiens
agggtttccttctcgctgattccttgtcttggtctccactagggccctggggggaggacgaggagtggacagacaaggcccggcgggtcatcatggagcgtatcggcctcgccactgcagggtaagggccctgtgcctgccctgttctactctctgg

SEQ ID NO: 876 - DNA, 150 bp, Homo Sapiens
aggtgtggtgttgcccaccagcccctcacccgcagtctgtctgcaggatgaagtcgctcacacagtcaccgagagccgggtcctccagaacaccaggcacccgttcctcactgtgagttgccctccccttcccagacagtgtgaggccag

SEQ ID NO: 877 - DNA, 153 bp, Homo Sapiens
tacccactccatttcccaccttctcccctcccaggccatgcacgaggggctgctgatgcccgtggtgaagtcagagggcggcgaggactacacgggagccactgtcatcgagcccctcaaagggtgaggccccaggctgggtgcagtttttac

SEQ ID NO: 878 - DNA, 151 bp, Homo Sapiens
ctgggctggcgtatgacggctgtcgctcctgcatttgcaggtgtctggcctgccaccgtgtctcaaggcggcctgcatacactcgggcatgaccaggaagcaacgggaatctgtcctgcagaaggtgggggcctcatgggcctaggggtga

SEQ ID NO: 879 - DNA, 143 bp, Homo Sapiens
ccgctcagtgtctctctcttgctctcgctctcgctctccccctctttctctctttctctctttttccgcgaggcctacacgacgccaggggtttgggtgcgtgttggggagggggagggggagcccatggggctccggagact

SEQ ID NO: 880 - DNA, 224 bp, Homo Sapiens
caggaagcctgtgttccgtacgacaatatggcggcgcttagttgcatgaaggcggaaactctgtgacttccggtccgtagtggggcctgcggtgggagtgggaaggaaggcggagggaaccatgcgaggttctgagaattgcggcgagggtcgcctcgagagacggtttctgaggtgggggccggacggtgcggggatcagaggcgggggcggggatatagagg

SEQ ID NO: 881 - DNA, 214 bp, Homo Sapiens
gaacttgccggttaagcaggcccccgtgtctctccctgttcccctgcagaaggccgggagtgtgtcaactgtggggccacagccacccctctctggcggcgggacggcaccggccactacctgtgcaatgcctgtggcctctaccacaagatgaatgggcagaaccgaccactcatcaagcccaagcgaagactggtaggagcgggcacaggtg

SEQ ID NO: 882 - DNA, 220 bp, Homo Sapiens
gtatcaacgctctgtgggtcgtgtgcgtgcgaggggggcgacgtaagggcgctccgcgagcccgtctctcctcgaatgaaaggaaacaacctccggcgacagagccccgctctcaggcactgctggagaaccgagaccgacttctttctctttaccctcattggcgcttctctcctgcagtccgcctctgggccctgccggtgagtccccacggaaccct

SEQ ID NO: 883 - DNA, 158 bp, Homo Sapiens
tgcttctcttccttctcccccaggagactgaggcatgccctgtggccctcacttccagacctgcaccgggtcctaggccagtaccttagggacactgcagccctgagcccggtgagtgtgcttccctcccctgtgcccaccaccaaccctgcctggta

SEQ ID NO: 884 - DNA, 297 bp, Homo Sapiens
ctttgtgtgccccgctccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacg

SEQ ID NO: 885 - DNA, 151 bp, Homo Sapiens
aggcctcttgtttcctccccaggcccctgagcctctgagctccttgaagtccatggcggaacgggcagccatcagctctggcattgaggaccctgtgccaacgctgcacctgaccgagcgaggtgagggacccaggatggtggggaagcag

SEQ ID NO: 886 - DNA, 166 bp, Homo Sapiens
ctatgggtgcccttctccacagatcatccagctgaccccggtgcctgtgagcacacccagcggcctggtgccgcccctgagcccagccacactccctggacccacctctcagcctcagaaggtcctgttgccctcctccaccaggtaattgcagctgagcccatac

SEQ ID NO: 887 - DNA, 151 bp, Homo Sapiens
gaggacctgtgggactctgcactgaggccctctctcccctccagggccgcctgcctgtgaagtggatggcgcccgaggccttgtttgaccgggtgtacacacaccagagtgacgtgtgagtcctgccggcggtcactgtcctaccccacaa

SEQ ID NO: 888 - DNA, 154 bp, Homo Sapiens
gggtgagactgacctctcttctcctgcccctgcctaggcccgcgatgctcccagcccggtgagacctgcctgaatggcgggaagtgtgaagcggccaatggcacggaggcctgcgtgtgagtaccacccctgcgggacctgttgctttgtcgag

SEQ ID NO: 889 - DNA, 343 bp, Homo Sapiens
ctgcagttcgcttgtgcccggcagcccgagctcgccatgatgcattgctcttactgggaccacgacagcaagaccggcgcgctgcattcgcgcctcgatctctgagagcccaccgcatgccggtgcagacggatgcgaggatgcagggacgcgcgacgccggccccggtcgcagccgacgacgccgccgccagcctgacctcacaccctctgggcccgcctctggagccagcgcccagggtccctctgtgctttttcgctttcctaagctcctgtcgctcctctttgtcccctcagtttatgtcctcctgtgctcacctccctgacctctgtgaccttgcact

SEQ ID NO: 890 - DNA, 199 bp, Homo Sapiens
cacccaccgctgtgttgcagctacctgaccgacgttgaccgcatcgccaccttgggctacctgcccacccagcaggacgtgctgcgggtccgcgtgcccaccaccggcatcatcgagtaccctttcgacctggagaacatcatcttccggtaccgcccgggccacagcaggcggggagggggcactgagaggctcattt

SEQ ID NO: 891 - DNA, 127 bp, Homo Sapiens
aggaagggagcctcaaaggccaaggccagccaggacaccccctgggatcacactgagcttgccacatccccaaggcggccgaaccctccgcaaccaccagcccaggtcagtctcagcccccagagag

SEQ ID NO: 892 - DNA, 206 bp, Homo Sapiens
gactctcctgtctccgctccctgccttgctcgcaggcagccacctggcgagtctgacatggctgtcagcgacgcgctgctcccatctttctccacgttcgcgtctggcccggcgggaagggagaagacactgcgtcaagcaggtgccccgaataacgtgagtatcgctccgggccgccgggaacgcccggtgggttttcgtgggtg

SEQ ID NO: 893 - DNA, 166 bp, Homo Sapiens
cctcctctgcgttcgacgcagcctccgcccggcctcccaggatgcagcgcgctggcgggaggtttggagcagatggataccgtatcgacgtggggcctccggtatgttgccgctgcgttgaggtaggatggggctggcgagtcttcccttcccaggacttcgcaga

SEQ ID NO: 894 - DNA, 169 bp, Homo Sapiens
actacatttcccaggaggcagcgggtctacgccgtcgccgtcgtcggagagcggagacgctgggcgcgctgtggggcgggggcgaggttcgggctggttgttccgttgcgagctgcagctgcgatctctgtggtaggcccaggtgagtgagcgcctctgatggaagtta

SEQ ID NO: 895 - DNA, 155 bp, Homo Sapiens
ccgtccgcgctacatactgcgcctgcgcaagggctgtggcccttttcccaccccctagcgccgctgggcctgcaggtctctgtcgagcagcggacgccggtctctgttccgcaggatggtgagtggatgcctcggtctcggggctttagatgcat

SEQ ID NO: 896 - DNA, 162 bp, Homo Sapiens
cagtggctcaggaaaccaaggggcccacacaggaaggagccgagtgggactttcctctcgctgcctcccggctctgcccgcccttcgaaagtccagggtccctgcccgctaggtaagagctggcgatgccgcagggctcggcccagacactgggggaggatg

SEQ ID NO: 897 - DNA, 153 bp, Homo Sapiens
gctgatcctccaccttccttcacccccacacagcccccccttgcctggacggaagcccgtgtgcaaatggaggtcgttgcacccagctgccctcccgggaggctgcctgcctgtgagtgcctggctcagagccaccagtgggccctgtgtgtg

SEQ ID NO: 898 - DNA, 220 bp, Homo Sapiens
gaacatggtgcgcaggttcttggtgaccctccggattcggcgcgcgtgcggcccgccgcgagtgagggttttcgtggttcacatcccgcggctcacgggggagtgggcagcgccaggggcgcccgccgctgtggccctcgtgctgatgctactgaggagccagcgtctagggcagcagccgcttcctagaagaccaggtaggaaaggccctcgaaaagtc

SEQ ID NO: 899 - DNA, 148 bp, Homo Sapiens
gtgtgttgggggatagcctcggtgtcagccatctttcaattgtgttcgcagccgccgccgcgccgccgtcgctctccaacgccagcgccgcctctcgctcgccgagctccagccgaaggagaaggggggtaagtttccccgtctgccc

SEQ ID NO: 900 - DNA, 193 bp, Homo Sapiens
cacgtctgcccctctctcccctgcggccagccctctacagccacaagcccgaggtggcccagtacacccacacgggcctgctcccgcagactatgctcatcaccgacaccaccaacctgagcgccctggccagcctcacgcccaccaagcaggtaaggtccaggcctgctggccctcccttggcctgtgacag

SEQ ID NO: 901 - DNA, 240 bp, Homo Sapiens
ggcagaagagaggcagacagactgacagacacgtagaccaacagtgcggccccagggttcgtccccagactcgctcgctcatttgttggcgactggggctcagcgcagcgaagcccgatgtggtccggaggcagtgggaaggcgcggggctgggaggccgcggcgggagggaggagcagccccggcaggctcaggtgaaacccccaccctgtccctcagccccctcctcctaaagacctg

SEQ ID NO: 902 - DNA, 348 bp, Homo Sapiens
tattaccggcagaaccagcagcgctggcagaactccatccgccactcgctgtccttcaatgactgcttcgtcaaggtggcacgctccccggacaagccgggcaagggctcctactggacgctgcacccggactccggcaacatgttcgagaacggctgctacttgcgccgccagaagcgcttcaagtgcgagaagcagccgggggccggcggcgggggcgggagcggaagcgggggcagcggcgccaagggcggccctgagagccgcaaggacccctctggcgcctctaaccccagcgccgactcgcccctccatcggggtgtgcacgggaagaccggccagctagag

SEQ ID NO: 903 - DNA, 198 bp, Homo Sapiens
cacccggttccatctacctttcccccaccccaggtctcctcttggctctgccaggagccggagccctgccaccctggctttgacgccgagagctacacgttcacggtgccccggcgccacctggagagaggccgcgtcctgggcagaggtgagggcgcgctgccggtgtccctgggcggagtagggaggggttggaaa

SEQ ID NO: 904 - DNA, 259 bp, Homo Sapiens
cgatgagggtctggccagcgccgcggcgcggggactagtggagaaggtgcgacagctcctggaagccggcgcggatcccaacggagtcaaccgtttcgggaggcgcgcgatccaggtagctggggccccagggcctcgccggcagggggcgcgcgaacgcggggcgcggcctcggcggatcggggctggaacctagatcgccgatgtagatttgtacaggagtctccgttggccggaggtgtgcattccacgcgtaaaa

SEQ ID NO: 905 - DNA, 150 bp, Homo Sapiens
cgctgctgccttgatgggctccgcggcccgagcgcctcttttcgggattaaaagcgccgccagctcccgccgccgccgccgtcgccagcagcgccgctgcagccgccgccgccggagaagcaaccgcgtaagtggcaacttttccctctt

SEQ ID NO: 906 - DNA, 150 bp, Homo Sapiens
actcactgaccctctcccttgacacagggcagccgctctggctctagctccagctccgggaccctctgggaccccccgggacccatgtgacccagcggcccctcgcgctgtaagtctcccgggacggcagggcagtgagggaggcgaggg

SEQ ID NO: 907 - DNA, 198 bp, Homo Sapiens
agcgaggacatctggaagaaattcgagctggtgccatcgccccccacgtcgccgccctggggcttgggtcccggcgcaggggacccggcccccgggattggtcccccggagccgtggcccggagggtgcaccggagacgaagcggaatcccggggccactcgaaaggctggggcaggaactacgcctccatcatacgc

SEQ ID NO: 908 - DNA, 152 bp, Homo Sapiens
caagaggccaatgagggggcagtgcccggcattatgcaacccgcctccccgcccgcccggtggagcttccactcggctgcgggctggagcggcggcgggcaggcgtgcggaggacactcctgcgaccaggtaggcatctctgcggccatcct

SEQ ID NO: 909 - DNA, 163 bp, Homo Sapiens
ctacctgtaactgggcctgttgctgtctcctagcacaaactctcagagcccatccccacccagcagttccattgcctacagcctcctgagtgccagctcagagcaggacaacccgtccaccagtggctgcaggtacgtcgggtgaggctggaggagaggtctg

SEQ ID NO: 910 - DNA, 296 bp, Homo Sapiens
gatggcgcctcagaagcacggcggtgggggagggggcggctcggggcccagcgcggggtccgggggaggcggcttcgggggttcggcggcggtggcggcggcgacggcttcgggcggcaaatccggcggcgggagctgtggagggggtggcagttactcggcctcctcctcctcctccgcggcggcagcggcgggggctgcggtgttaccggtgaagaagccgaaaatggagcacgtccaggctgaccacgagcttttcctccaggcctttgagagtgagtgtgtgcgaggctttg

SEQ ID NO: 911 - DNA, 283 bp, Homo Sapiens
gggctccgtagacgctttccgcatcactctccttcctcgggctgccgggagtcccgggacctggcggggccggcatgacgggcttctcgggggcccgccgcacgcccggcagcctccggagacgcgcgccgagcccggctcccacggcctctgaggctcggcggggctgcggctgcctggcgggcgggctccggagctttcctgagcggcattagcccacggcttggcccggacgcgaccaaaggctcttctggagaagcccagagcactgggcaatcgttac

SEQ ID NO: 912 - DNA, 162 bp, Homo Sapiens
ctgccttctcccctgaagagagacgcggggggaggggggtgcggcgagcggccccgctctctccccaccgctccgctcgcaccccagtgtaatgagggtcaccccctccccccagctggcccgggagggggcgcggggcacggtaactagtgcgctggggtg

SEQ ID NO: 913 - DNA, 51 bp, Artificial Sequence
  synthesized
  misc_feature (51)..(51): labelled with inverted dT
aagagcgtcgtgtagggaaagagtgtagatctcggtggtcgccgtatcatt

SEQ ID NO: 914 - DNA, 65 bp, Artificial Sequence
  synthesized
  misc_feature (35)..(41): n is a, c, g, or t
  misc_feature (65)..(65): labelled with inverted dT
cctcagcaagagcacacgtctgaactccagtcacnnnnnnnatctcgtatgccgtcttctgcttg